(19) United States
(12) Patent Application Publication
(10) Pub. No.: US 2018/0111972 A1
(43) Pub. Date: Apr. 26, 2018

(54) HYBRID CHIMERA POLYPEPTIDES AS DUAL INHIBITORS OF VASCULAR ENDOTHELIAL GROWTH FACTOR RECEPTOR AND PLATELET-DERIVED GROWTH FACTOR RECEPTOR

(71) Applicant: The Board of Trustees of the Leland Stanford Junior University, Stanford, CA (US)

(72) Inventors: Jennifer R. Cochran, Stanford, CA (US); Cheuk Lun Leung, Ocean Springs, MS (US); Shiven Kapur, Palo Alto, CA (US); Mark S. Blumenkranz, Portola Valley, CA (US)

(21) Appl. No.: 15/540,167

(22) PCT Filed: Jan. 15, 2016
(86) PCT No.: PCT/US16/13674

§ 371 Date: Jun. 27, 2017

Related U.S. Application Data
(60) Provisional application No. 62/104,612, filed on Jan. 16, 2015.
(60) Provisional application No. 62/263,518, filed on Dec. 4, 2015.

(51) Int. Cl. C07K 14/49; C07K 14/475; A61P 35/00; A61P 17/06; A61P 27/10; A61P 27/02; A61P 19/02; G01N 33/68

(52) U.S. Cl. CPC C07K 14/49; C07K 14/475; A61P 35/00; A61P 17/06; A61K 38/00; A61P 27/02; A61P 19/02; G01N 33/6893; C07K 2319/74; A61P 27/10

(57) ABSTRACT

Polypeptides comprising hybrid VEGF and PDGF sequences are provided. The polypeptides are useful in inhibition of angiogenesis and treatment of diseases characterized by pathologic neovascularization.

### CROSS-REFERENCE

This application is a 371 application and claims the benefit of PCT Application No. PCT/US2016/013674, filed Jan. 15, 2016, which claims benefit of U.S. Provisional Patent Application Ser. No. 62/104,612, filed Jan. 16, 2015, and U.S. Provisional Patent Application Ser. No. 62/263,518, filed Dec. 4, 2015, each of which is incorporated by reference herein in its entirety.

### GOVERNMENT RIGHTS

This invention was made with Government support from contract W81XWH-11-1-0364 awarded by the Department of Defense. The Government has certain rights in the invention.

### FIELD OF THE INVENTION

The present invention relates to the field of angiogenesis-related diseases and their diagnosis, characterization and treatment.

### BACKGROUND OF THE INVENTION

Angiogenesis, the process of new blood vessel formation from preexisting vasculature, plays critical roles in both normal physiological processes such as wound healing, pregnancy, tissue regeneration and in the pathogenesis of cancer, rheumatoid arthritis, and diabetic microvascular disease (see Carmeliet P (2005), Nature 438, pp. 932-936), and is regulated by a large number of pro- and antiangiogenic cytokines and growth factors (Ferrara N (2000), Curr Opin Biotechnol 11, pp. 617-624). During adulthood, most blood vessels remain quiescent and angiogenesis occurs only in the cycling ovary and in the placenta during pregnancy.

However, when angiogenic growth factors are produced in excess of angiogenesis inhibitors, endothelial cells are stimulated to proliferate. A number of angiogenic growth factors have been described to date among which vascular endothelial growth factor (VEGF) appears to play a key role as a positive regulator of physiological and pathological angiogenesis (Brown et al. (1997) in “Control of Angiogenesis” (Goldberg and Rosen, eds.), Birkhauser, Basel, pp. 233-269; Thomas K A (1996), J Biol Chem 271, pp. 603-606; Neufeld et al. (1999), FASEB J 13, pp. 9-22).

Platelet-derived growth factor (PDGF) and its receptor (PDGFR) have been identified as important disease targets in cancer as their overexpression and aberrant signaling play a role in angiogenesis and metastasis. Blocking PDGFR signaling would inhibit these processes, slowing disease progression. Despite their important role in cancer, there are currently no FDA approved drugs that specifically target PDGF or PDGFR. Current anti-angiogenesis therapies have focused on targeting the vascular endothelial growth factor (VEGF) and its receptor (VEGFR). However, resistance to these therapies arises when alternate signaling pathways for angiogenesis, such as those mediated by PDGFR, evolve to compensate for the decrease in VEGFR.

Thus, it is important to develop additional inhibitors that can be used in combination therapy to target multiple pathways and more completely block angiogenesis. The present invention addresses this need.

### Publications

US patent application 20140315804 discloses fusion proteins comprising PDGF and VEGF binding portions. European patent no. EPI919944 discloses modified VEGF and PDGF proteins with improved angiogenic properties.

The sequences of human VEGF and PDGF are known and publically available, for example at Genbank, accession number AAH65522.2; and EMBL accession number CAA45383.1.

Dual inhibitors of VEGFR and PDGFR include the broad inhibitor of receptor protein-tyrosine kinases, sunitinib (see Roskoski et al. (2007) Biochem Biophys Res Commun).

### SUMMARY OF THE INVENTION

Compositions are provided of hybrid chimera polypeptides (which may be referred to herein as hybrid polypeptides or chimera polypeptides) that are dual inhibitors of human PDGFR and VEGFR. PDGF and VEGF activate their receptors by binding to two PDGFR or two VEGFR molecules, respectively, which induces dimerization of the receptors and activation of downstream signaling. Accordingly, ligand binding to a single receptor does not activate signaling. The hybrid polypeptides are composed of PDGF residues on one pole and VEGF residues on the opposite pole; therefore, they bind to a single VEGFR but not PDGFR at one pole; and bind to a PDGFR but not VEGFR at the other pole. By binding to only one of each VEGFR and PDGFR, both receptors are coordinately blocked from dimerization and activation, thereby inhibiting both pathways.

The hybrid polypeptides of the invention comprise two fused polypeptide chains, in which each polypeptide chain is a hybrid of alternating VEGF and PDGF sequences. This particular strategy was necessitated by the nature of the receptor binding interface of each ligand which is composed of noncontiguous ligand segments; therefore, a hybrid polypeptide was created by alternating patches of PDGF and VEGF to preserve the PDGF-PDGFR and VEGF-VEGFR binding interfaces at each pole. Using molecular modeling, breakpoints at regions of structural overlap and/or conserved residues were used to create a hybrid protein that can fold properly and adopt the native PDGF and VEGF structures at each pole. In some embodiments of the invention, the hybrid polypeptide comprises one or more amino acid substitutions that provide for increased affinity for one or both of VEGFR and PDGFR.

The hybrid polypeptide comprises two engineered polypeptide chains, which may be referred to herein as hybrid chain 1 (HC1) and hybrid chain 2 (HC2), fused through a linker. The amino acid sequences may be discussed with reference to a wild-type human PDGF-B polypeptide, provided herein as SEQ ID NO: 1; and a wild-type human VEGF-A polypeptide, provided herein as SEQ ID NO: 2. Residues 7-104 from mature PDGF and residues 13-109 from mature VEGF were used in the construction of the polypeptide compositions of the invention; residue numbering has been chosen to be consistent with standard nomenclature and with respect to the provided reference sequences. It will be understood by one of skill in the art that the naming is arbitrary and that the orientation of the two chains may be reversed, i.e. the polypeptide may be organized as HC1-linker-HC2; or as HC2-linker-HC1.

The hybrid polypeptides of the invention bind coordinately to VEGF and PDGF receptors but do not induce receptor activation, thereby simultaneously antagonizing VEGF- and PDGF-stimulated receptor autophosphorylation and proliferation of cells.

Compositions include one or more hybrid polypeptide(s) of the invention, which may be provided as a single species or as a cocktail of two or more polypeptides, usually in combination with a pharmaceutically acceptable excipient. Such compositions optionally comprise one or more additional therapeutic agents. Pharmacologic compositions comprise one or more polypeptides of the invention and a pharmaceutically acceptable excipient. Compositions can be provided for topical or systemic use. In some embodiments, the pharmaceutical composition is a topical composition. In some embodiments, the pharmaceutical composition is a locally injected composition into the skin, ocular tissue, cerebrospinal fluid, tumor, joint space, etc. In some embodiments, the pharmaceutical composition is a systemic composition delivered orally or intravenously. In some embodiments, the pharmaceutical composition is an eye drop. In some embodiments, the pharmaceutical composition is formulated as an ophthalmically acceptable solution, cream or ointment. Ophthalmic compositions of the invention can be formulated for non-surgically treating a disorder characterized by neovascularization of the external surface of the eye, including the cornea and bulbar conjunctiva, in an subject in need thereof. In some embodiments the composition is formulated for preventing recurrence of a disorder characterized by neovascularization of the external surface of the eye, including the cornea and bulbar conjunctiva, in a subject in need thereof. In some embodiments, the composition is formulated for intraocular injection, subconjunctival injection, or periocular injection.

In some embodiments the polypeptide of the invention is conjugated to a functional moiety, e.g. a detectable label such a fluorescent label, a detectable label such as an isotopic label; a cytotoxic moiety, and the like, which may find use in imaging, quantitation, therapeutic purposes, etc. In some embodiments, the hybrid polypeptide of the present invention further comprises a toxin. In some embodiments, the toxin is selected from the group consisting of a Pseudomonas exotoxin (PE), a Diphtheria toxin (DT), ricin toxin, abrin toxin, anthrax toxins, shiga toxin, botulism toxin, tetanus toxin, cholera toxin, maitotoxin, palytoxin, ciguatoxin, textilotoxin, batrachotoxin, alpha conotoxin, taipoxin, tetrodotoxin, alpha tityustoxin, saxitoxin, anatoxin, microcystin, aconitine, exfoliatin toxins A, exfoliatin B, an enterotoxin, toxic shock syndrome toxin (TSST-I), Y. pestis toxin and a gas gangrene toxin. In some embodiments, the toxin is attached to the N-terminus of the polypeptide. In some embodiments, the toxin is attached to the C-terminus of the polypeptide. In some embodiments, the toxin is attached to the PDGF chain, the VEGF chain, or both.

Compositions also include nucleic acids encoding such polypeptides, which may be provided in a vector suitable for delivery to a mammalian cell. In some aspects, the invention provides a vector comprising a nucleotide sequence encoding any of the hybrid proteins disclosed herein. In some embodiments, the vector is a viral vector.

In some embodiments, the invention provides a method of producing a hybrid protein, comprising culturing a host cell comprising a nucleic acid encoding any of the hybrid proteins disclosed herein under a condition that produces the hybrid protein, and recovering the hybrid protein produced by the host cell. In certain specific embodiments, the host cell is a mammalian or microbial cell, although other expression hosts also find use.

In other embodiments, amino acid variants of PDGF are provided, in which the affinity for the PDGFR is enhanced relative to the wild-type protein. In some such embodiments, the variant proteins comprise one or both of the amino acid substitutions I77F and K98R with reference to SEQ ID NO: 1. Such changes are also usefully incorporated into the hybrid proteins, as described above.

In some embodiments the hybrid polypeptides of the invention are used for the treatment or prevention of a disease such as an ocular disease, an inflammatory disease, an autoimmune disease, or cancer. The polypeptides of the invention also find use for imaging normal tissue, abnormal tissue, precancerous tissue, cancer, and tumors. In other embodiments methods are provided for diagnosis of precancerous tissue, cancer, and tumors.

In another embodiment, the invention provides a method of delivering a hybrid protein to a subject comprising administering an effective amount of any of the hybrid proteins disclosed herein to the subject. In some embodiments, the subject has macular degeneration or proliferative diabetic retinopathy. In a further embodiment, the macular degeneration is wet age-related macular degeneration or dry age-related macular degeneration. In some embodiments herein, the hybrid protein is administered by intravitreal injection to the subject. In some embodiments, the subject has cancer. In some embodiments, the subject has rheumatoid arthritis, osteoarthritis, or asthma. In some embodiments, the subject has uveitis or corneal neovascularization. In some embodiments, the subject has a fibrovascular growth, including but not limited to pterygium.

In some embodiments, methods are provided for non-surgically treating a disorder characterized by neovascularization of the external surface of an eye, including the cornea and bulbar conjunctiva, of a subject in need thereof, comprising administering to the subject an effective amount of a pharmaceutical composition comprising a hybrid polypeptide of the present invention. In some embodiments, methods are provided for preventing recurrence of neovascularization of the external surface of an eye, including the cornea and bulbar conjunctiva, of a subject in need thereof, comprising administering to the subject an effective amount of a pharmaceutical composition comprising a hybrid polypeptide of the present invention.

In some embodiments, the method comprises administering an additional therapeutic agent. In some embodiments, the additional therapeutic agent is selected from the group consisting of an antibody, polypeptide, nucleotide, a small molecule, and combinations thereof. In some embodiments, the additional therapeutic agent is an inhibitor of a VEGF, an inhibitor of a PDGF, an inhibitor of an ANG, or an inhibitor of a FGF, or associated receptors. In some embodiments, the additional therapeutic agent is an inhibitor of an integrin, or an inhibitor of a MMP, or an inhibitor of prostate specific membrane antigen (PSMA). In some embodiments, the additional therapeutic agent is selected from the group consisting of: mitomycin C (MMC), 5-fluorouracil (5-FU), loteprednol etabonate (LE), oral doxycycline, dipyridamole, and dobesilate. In some embodiments, the additional therapeutic agent is an anti-inflammatory steroid. In some embodiments, the additional therapeutic agent is non-steroidal anti-inflammatory agent. In some embodiments, the additional therapeutic agent is an antibody or small molecule inhibitor of VEGF signaling. In some embodiments, the additional therapeutic agent binds, traps, scavenges or otherwise deters the effect of VEGF that has already been produced. The additional therapeutic agent can be formulated in the pharmaceutical composition, including ophthalmic compositions, with the hybrid polypeptide of the invention, or can be administered in a separate formulation.

In some embodiments, the disorder characterized by neovascularization of the external surface of the eye is pterygium. In some embodiments, the pterygium is chronic pterygium. In some embodiments, the pterygium is recurrent pterygium. In some embodiments, the disorder characterized by neovascularization of the external surface of the eye is pannus. In some embodiments, the disorder characterized by neovascularization of the external surface of the eye is corneal neovascularization. In some embodiments, the disorder characterized by neovascularization of the external surface of the eye is pinguecula. In some embodiments, the disorder characterized by neovascularization at the limbus of the cornea caused by contact lens overwear. In some embodiments, the disorder has not healed within one month of a surgical intervention. In some embodiments, the hybrid polypeptide of the present invention is administered during or after a surgical intervention or debridement.

### BRIEF DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures..

FIG. 1. Alignment of native VEGF and PDGF with hybrid sequences.

FIG. 2A-2B. Ligand-based antagonists of PDGFR and VEGFR. FIG. 2A PDGF and VEGF are dimers that have two binding sites to PDGFR and VEGFR, respectively. FIG. 2B A hybrid PDGF/VEGF protein has PDGF on one pole and VEGF on the other pole, which can bind to one PDGFR or one VEGFR, acting as an antagonist by preventing receptor dimerization and activation.

FIG. 3. Structural alignment of PDGF-B and VEGF-A. Comparison of the PDGF-B and VEGF-A homodimer crystal structures [pdb: 3MJG, 3V2A] reveals significant structural homology despite highly variable primary sequences.

FIG. 4. PDGFR and VEGFR binding to PDGF/VEGF hybrid constructs displayed on yeast. Five hybrid designs were displayed on the surface of yeast and tested for their ability to bind soluble PDGFR or VEGFR2. All five constructs were well expressed on yeast; one construct (PV1) was able to bind to both PDGFR and VEGFR. scPDGF and scVEGF were used as controls.

FIG. 5. PDGFR and VEGFR binding to second-generation PDGF/VEGF hybrid constructs displayed on yeast. Three additional hybrid designs were displayed on the surface of yeast and tested for their ability to bind soluble PDGFRβ or VEGFR2. All three constructs were well expressed on yeast and bound to both receptors; PV6 displayed the highest expression and binding to PDGFR and VEGFR.

FIG. 6. Binding of PV6 to BJ-5ta and NIH/3T3 cells. Various concentrations of soluble PV6 were titrated with BJ-5ta and NIH/3T3 cells. K_{d} values of 0.45 nM and 0.34 nM were observed for BJ-5ta and NIH/3T3, respectively.

FIG. 7. Binding titration of scPDGF. Various concentrations of scPDGF were titrated with NIH/3T3 and BJ-5ta cells Specific binding was observed, with K_{d} of 2.5±1.5 pM on NIH/3T3 and 1.5±0.2 pM on BJ-5ta cells.

FIG. 8. Stimulation of DiscoveRx PathHunter PDGFRβ cells. DisccoveRx PathHunter PDGFRβ cells were stimulated with scPDGF and wt PDGF-BB at various concentrations. Activated receptor was detected using the DiscoveRx chemiluminescent substrate and analyzed on a microplate reader. Similar dose response curves were observed for both scPDGF and wt PDGF-BB, with EC₅₀ of 140 pM and 250 pM, respectively.

FIG. 9. Inhibition of phosphorylation in DiscoveRx PathHunter PDGFRb cells. DisccoveRx PathHunter PDGFRb cells were stimulated with scPDGF at various concentrations. PV6 inhibited stimulation by 0.1 nM scPDGF with IC₅₀ of 19 nM.

FIG. 10. Sorting scheme for error-prone PCR library for increased binding to PDGFR or VEGFR. Library of hybrid PDGF/VEGF mutants were generated by error-prone PCR, displayed on the surface of yeast, and screened for binding to soluble PDGFR or VEGFR by FACS. Two parallel screens were performed, one for PDGFR and one for VEGFR. To increase the screening stringency, the concentration of soluble receptor was decreased with each round of sorting, followed by “off-rate” screens were performed in which binding reactions were sorted 4 to 48 hours after removal of unbound receptor to isolate hybrid PDGFNEGF variants with decreased kinetic off-rates that retained binding to PDGFR or VEGFR.

FIG. 11. Sorting scheme for StEP library for increased binding to PDGFR and VEGFR. Library generated using StEP were sorted for increased binding to both PDGFR and VEGFR. Mutations for the PDGFR and VEGFR sorts were combined by in vitro recombination to create two libraries PV_{shuf} and V_{shuf}. Two rounds of equilibrium sorts were followed by two rounds of “off-rate” sorts, alternating between PDGFR and VEGFR binding for each sort.

FIG. 12. Binding affinity of 4-22 RF compared to the PDGFR and VEGFR affinity-matured clones. 4-22 RF was created by combining mutations identified from PDGFR sorts (RF) and VEGFR sorts (4-22). Compared to the mutants affinity-matured to each receptor, the combined mutant displays almost identical binding to PDGFR and VEGFR.

FIG. 13A-13D. SDS-PAGE of PDGFNEGF variants. FIG. 13A PV6 showed multiple bands when reduced, but only one band in non-reducing conditions. FIG. 13BR137A and R133S mutations to remove protease site in the PDGF part of the molecule. FIG. 13C PV_{GS} removed the extra bands present in PV_{AM-S}. FIG. 13D Comparison of all constructs tested.

FIG. 14. Binding of PV RF to BJ-5ta and NIH/3T3 cells. Various concentrations of soluble PV RF were titrated with BJ-5ta and NIH/3T3 cells. K_{d} values of 7.1 pM and 15 pM were observed for BJ-5ta and NIH/3T3, respectively.

FIG. 15. Binding of PV R32A to BJ-5ta cells. Various concentrations of soluble PV R32A were titrated with BJ-5ta cells. A K_{d} value of 10.9 pM was observed.

FIG. 16. Binding of PV R32A to HUVECs. Various concentrations of soluble PV R32A and scVEGFwt were titrated with HUVECs. K_{d} values of 0.2 nM and 1.5 nM were observed for PV R32A and scVEGFwt, respectively.

FIG. 17. Inhibition of phosphorylation in DiscoveRx PathHunter PDGFRβ cells. DisccoveRx PathHunter PDGFRβ cells were stimulated with scPDGF at various concentrations. 4-22 RF and PV R32A inhibited stimulation by 0.1 nM scPDGF with IC₅₀ of 0.51 and 0.37 nM, respectively.

FIG. 18A-18D. Western blot analysis of VEGF and PDGF stimulation. FIG. 18A BJ-5ta cells were stimulated with 0.5 nM PDGF-BB and various concentrations of PDGFNEGF chimera. FIG. 18B HUVECs cells were stimulated with 0.5 nM VEGF₁₆₅ and various concentrations of PDGF/VEGF chimera. FIG. 18C PDGF-BB dose-response stimulation of PDGFR phosphorylation in BJ-5ta cells. FIG. 18D VEGF₁₆₅ dose-response stimulation of VEGFR2 phosphorylation in HUVECs. PV_{AM-S} does not stimulate phosphorylation of VEGFR2 or PDGFRβ.

FIG. 19A-19C. Analysis of binding to PDGFR for clones isolated from random mutagenesis library sorts. FIG. 19A Binding to PDGFR-Fc showed that K87R and I182F have higher maximum levels of binding compared to PV6, but not a significant improvement in Kd. FIG. 19B Binding to monovalent PDGRβ-His showed that K87R and I182F had improved binding compared to PV6. FIG. 19C Kd of PV6, K87R, and I182F binding to PDGRβ-Fc and PDGRβ-His.

FIG. 20. Analysis of binding to VEGFR for clones isolated from random mutagenesis library sorts. Binding to VEGFR2-Fc showed that the two dominant clones after sort 6, 6-5 and 6-2, had improved binding to VEGFR2-Fc compared to PV6.

FIG. 21A-21C. Binding titrations of yeast displayed variants isolated from Vshuf library with various mutations. Yeast displayed variants were tested for binding to (A) VEGFR2-Fc or (B) VEGFR2-His. (C) Mutations of the variants compared to PV6.

FIG. 22. Binding titrations of yeast displayed variants binding to soluble PDGFRβ and VEGFR2. Combining mutations for improved PDGFR binding (PV_{RF}) with mutations for improved VEGFR binding (V₄₋₂₂) created a variant with high affinity to both PDGRβ-His and VEGFR2-His (PV_{AM}).

FIG. 23A-23B. Simultaneous binding of PDGFR and VEGFR. Comparison of FIG. 23A PDGRβ-Fc and FIG. 23B VEGFR2-Fc binding levels after pre-incubation with the other receptor.

FIG. 24. Binding of yeast-displayed PDGF/VEGF variants to PDGFR and VEGFR. Equilibrium binding titrations of PDGRβ-His and VEGFR2-His to PDGF/VEGF variants.

FIG. 25A-25D. Binding of PDGF/VEGF chimera to PDGFR and VEGFR expressing cells. Equilibrium binding titrations of recombinantly expressed PDGF/VEGF chimera to FIG. 25A BJ-5ta human fibroblasts expressing PDGFR, FIG. 25B HUVECs human endothelial cells expressing VEGFR, FIG. 25C NIH/3T3 mouse fibroblasts expressing PDGFR, and FIG. 25D SVR mouse endothelial cells expressing VEGFR.

FIG. 26A-26C. Kinetic exclusion assay standard K_{d} experiments for PDGFR. KinExA binding data and n-curve analysis fits for equilibrium binding experiments to PDGRβ-His. FIG. 26A scPDGF at 25 nM and 1 nM. FIG. 26B PV6 at 10 nM and 1 nM. FIG. 26C PV_{GS} at 1 nM and 25 pM. High concentration curves are shown in red, and low concentration curves in blue. Solid lines are global fits from n-curve analysis of both curves.

FIG. 27A-27C. Kinetic exclusion assay “Kinetics Injection” experiments for PDGFR. KinExA binding data and fits for kinetics experiments. FIG. 27A 1 nM scPDGF with 5.01 s pre-incubation time. FIG. 27B 1 nM PV6 with 9.67 s pre-incubation time. FIG. 27C 1 nM PV_{GS} with 20.87 s pre-incubation time. Data and curve fit shown in red. Theoretical curve at equilibrium shown as green dotted line.

FIG. 28A-28B. Kinetic exclusion assay standard Kd experiments for VEGFR. KinExA binding data and n-curve analysis fits for equilibrium binding experiments to VEGFR2-His. FIG. 28A PV_{GS} at 1 nM and 25 pM. FIG. 28B PV6 at 10 nM. High concentration curve is shown in red, and low concentration curve in blue. Solid lines are global fits from n-curve analysis of both curves.

FIG. 29A-29B. Kinetic exclusion assay “Kinetics Injection” experiments for VEGFR. KinExA binding data and fits for kinetics experiments. FIG. 29A 1 nM PV_{GS} with 35.62 s pre-incubation time. FIG. 29AB 30 nM PV6 with 20.87 s pre-incubation time. Data and curve fit shown in red. Theoretical curve at equilibrium shown as green dotted line.

FIG. 30A-30B. Inhibition of PDGF-stimulated phosphorylation in DiscoveRx PathHunter PDGFRβ reporter cell line. FIG. 30A Stimulated with PDGF-BB. FIG. 30B Cells were stimulated with 0.5 nM PDGF-BB and various concentrations of PDGF/VEGF chimera. Phosphorylated PDGFR was detected with PathHunter reagent and luminescence readout.

FIG. 31A-31D. Inhibition of PDGF- and VEGF-stimulated cell proliferation. FIG. 31A BJ-5ta cells were stimulated with PDGF-BB. FIG. 31B BJ-5ta cells were stimulated with 5 nM PDGF-BB and various concentrations of PDGF/VEGF chimera. FIG. 31C HUVECs were stimulated with VEGF₁₆₅. FIG. 31D HUVECs were stimulated with 0.5 nM VEGF₁₆₅ and various concentrations of PDGF/VEGF chimera. Cell proliferation was detected using alamarBlue.

FIG. 32. Sequences of engineered PDGF/VEGF chimera. PV6 is shown with sequences derived from PDGF-B in green, VEGF-A in blue, conserved residues in yellow, and linker in gray. Mutations from PV6 that conferred improved binding to PDGFR or VEGFR and improved stability are highlighted in red. The sequences shown in the figure are full length proteins, where two chains are joined by a linker. PV6 is comprised of PV6 chains 1 and 2, joined by GSTSGSGKSSEGKG linker. PV AM is comprised of 4-22 RF chains 1 and 2, joined by GSTSGSGKSSEGKG linker, which may also be referred to as 4-22 RF. PV AM-S is comprised of PV R32A chains 1 and 2, joined by GSTSGSGKSSEGKG linker, which may also be referred to PV R32A. PV GS is comprised of PV R32A chains 1 and 2, joined by (GGGGS)4 linker.

FIG. 33A-33B. Binding titrations of recombinantly expressed PDGFR. Titration curves with yeast-displayed scPDGF with FIG. 33A PDGRβ-Fc and FIG. 33B PDGRβ-His.

FIG. 34. Inhibition of PDGFR phosphorylation by PDGRβ-Fc. Western blot analysis of phosphorylated PDGFRβ and phosphotyrosine in NIH/3T3 cell lysates after stimulation with 150 pM scPDGF with various concentration of PDGRβ-Fc. β-tubulin was used as a loading control.

FIG. 35. Antagonism of scPDGF stimulation of DiscoveRx PathHunter PDGFRβ cells by PDGRβ-Fc. PDGRβ-Fc was tested for its ability to inhibit PDGFR stimulation by 100 pM scPDGF.

FIG. 36. Antagonism of scPDGF stimulation of cell proliferation by PDGRβ-Fc. NIH/3T3 cells were stimulated with 100 pM scPDGF and various concentrations of PDGFRβ-Fc, and cell proliferation was measured using alamarBlue.

FIG. 37. exemplifies immunohistochemical staining of von Willebrand Factor (vWF) and VEGFR2 in human pterygium.

FIG. 38. Immunohistochemical staining of CD31, and PDGFR-β in human pterygium.

### DETAILED DESCRIPTION OF THE EMBODIMENTS

VEGFR and PDGFR are critical effectors of tumor angiogenesis with broad clinical utility for the early detection as well as treatment of many solid cancers. Therapeutic and diagnostic agents that selectively inhibit VEGFR as well as PDGFR are beneficial for treating angiogenesis-related disorders, in particular neoplasia and tumor metastasis. In addition to cancer, other proliferative diseases characterized by excessive neovascularization, e.g. psoriasis, age-related macular degeneration, diabetic retinopathy, rheumatoid arthritis, and the like, are treated with an effective dose of polypeptides of the invention, where the dose is effective at inhibiting angiogenesis.

SEQUENCE LISTING

| | |
|---|---|
| SEQ ID NO: 1 | wt human PDGF-B |
| SEQ ID NO: 2 | wt human VEGF-A |
| SEQ ID NO: 3 | PV1 |
| SEQ ID NO: 4 | PV2 |
| SEQ ID NO: 5 | PV3 |
| SEQ ID NO: 6 | PV4 |
| SEQ ID NO: 7 | PV5 |
| SEQ ID NO: 8 | PV6 chain 1 |
| SEQ ID NO: 9 | PV6 chain 2 |
| SEQ ID NO: 10 | PV7 chain 1 |
| SEQ ID NO: 11 | PV7 chain 2 |
| SEQ ID NO: 12 | PV8 chain 1 |
| SEQ ID NO: 13 | PV8 chain 2 |
| SEQ ID NO: 14 | PV K87R chain 1 |
| SEQ ID NO: 15 | PV I182F chain 2 |
| SEQ ID NO: 16 | 3-3 chain 1 |
| SEQ ID NO: 17 | 3-3 chain 2 |
| SEQ ID NO: 18 | 4-22 chain 1 |
| SEQ ID NO: 19 | 4-22 chain 2 |
| SEQ ID NO: 20 | 5-5 chain 1 |
| SEQ ID NO: 21 | 5-5 chain 2 |
| SEQ ID NO: 22 | 4-22 RF chain 1 |
| SEQ ID NO: 23 | 4-22 RF chain 2 |
| SEQ ID NO: 24 | PV R32A chain 1 |
| SEQ ID NO: 25 | PV R32A chain 2 |
| SEQ ID NO: | |

### Definitions

“VEGF” is a secreted disulfide-linked homodimer that selectively stimulates endothelial cells to proliferate, migrate, and produce matrix-degrading enzymes, all of which are processes required for angiogenesis, the formation of new blood vessels from existing vessels. In addition to being the only known endothelial cell specific mitogen, VEGF is unique among angiogenic growth factors in its ability to induce a transient increase in blood vessel permeability to macromolecules. The term “VEGF” as used herein refers to proteins that are also known in the literature as “VEGF-A”, i.e. the VEGF isoforms containing 121, 145, 165, 189 or 206 amino acid residues as described herein. In particular, a reference VEGF-A sequence is provided as SEQ ID NO: 2, to which reference will be made with respect to the numbering of specific amino acids.

VEGF signaling is mediated largely via two homologous, endothelium-specific tyrosine kinase receptors, VEGFR1 (Flt-1 aka fms-like tyrosine kinase 1) and VEGFR2 (Flk-1/KDR aka kinase domain receptor) whose expression is highly restricted to cells of endothelial origin (de Vries et al. (1992), Science 255, pp. 989-991; Millauer et al. (1993), Cell 72, pp. 835-846; Terman et al. (1991), Oncogene 6, pp. 519-524). Both receptors have an extracellular domain consisting of seven IgG-like domains, a transmembrane domain and an intracellular tyrosine kinase domain. The affinity of VEGFR1 for VEGF (Kd=1-20 pM) is higher compared to that of VEGFR2 (Kd=50-770 pM) (Brown et al. (1997) in “Control of Angiogenesis” (Goldberg and Rosen, eds.), Birkhauser, Basel, pp. 233-269; de Vries et al. (1992), Science 255, pp. 989-991; Terman et al. (1992) Biochem Biophys Res Commun 187, pp. 1579-1586).

VEGFR2 is the principal mediator of the mitogenic, angiogenic and permeability-enhancing effects of VEGF and, thus, a major factor in tumor angiogenesis; as a consequence, VEGFR2 overexpression can be observed on endothelial cells in tumor-associated angiogenic vasculature in many cancers (Tucker GC (2006), Curr Oncol Rep 8, pp. 96-103; Parker et al. (2005), Protein Eng Des Sel 18, pp. 435-44; Boesen et al. (2002), J Biol Chem 277, pp. 40335-41; Siemeister et al. (1998), Proc Natl Acad Sci U S A 95, pp. 4625-9; Cai et al. (2005), Biotechniques 39, pp. S6-S17; Haubner R (2006), Eur J Nucl Med Mol Imaging 33 Suppl 1, pp. 54-63).

The VEGF homodimer contains two receptor binding interfaces lying on each pole of the molecule. Each of the two binding interfaces is typically able to contact one receptor monomer (either VEGFR1 or VEGFR2), thereby inducing receptor dimerization and activation.

“PDGF”, Platelet-derived growth factor, is one of the numerous growth factors, or proteins that regulate cell growth and division. In particular, it plays a significant role in angiogenesis, which is the growth of blood vessels from already-existing blood vessel tissue. It does so by recruiting perivascular cells to the immature tumor vasculature where they interact with endothelial cells to stabilize the vessels resulting in mature blood vessels (Andrae et al. (2008), Genes and Development 22, pp. 1276-1312).

There are at least four members of the PDGF family of proteins that regulate the PDGF signaling pathway, specifically PDGF-A, PDGF-B, PDGF-C, and PDGF-D. These four PDGFs assemble into disulfide-linked dimers via homo- or heterodimerization. At least five different dimeric isoforms of PDGF have been described to date and include PDGF-AA, PDGF-BB, PDGF-CC, PDGF-DD, and PDGF-AB, all of which bind to PDGF receptors (PDGFRs) to activate the PDGF signaling pathway. In particular, a reference PDGF-B sequence is provided as SEQ ID NO: 1, to which reference will be made with respect to the numbering of specific amino acids.

The PDGF homodimer or heterodimer contains two receptor binding interfaces lying on each pole of the molecule. Each of the two binding interfaces is typically able to contact one receptor monomer (either PDGFR-α or PDGFR-β), thereby inducing receptor dimerization and activation.

There are at least two identified PDGFRs, PDGFR-α and PDGFR-β. Each PDGFR has an extracellular region, a transmembrane domain, and an intracellular region having intracellular tyrosine kinase activity. PDGFRs can dimerize to form the homodimers PDGFR-α/PDGFR-α or PDGFRβ/PDGFR-β and the heterodimer PDGFR-α/PDGFR-β. Each of these PDGFR dimer forms recognize different dimeric isoforms of PDGF. For example, PDGFR-α/PDGFR-α recognizes PDGF-AA, AB, BB and CC ligands, PDGFR-α/PDGFR-β recognizes PDGF-AB, BB, CC, and DD, and PDGFR-β/PDGFR-β recognizes PDGF-BB and DD. Deletion mutagenesis of the PDGF-AA and -BB binding sites have been mapped to amino acids 1-314 of PDGFR-α while the PDGF-BB binding sites have been mapped to amino acids 1-315 of PDGFR-β. The extracellular region of these PDGFRs, which mediate binding to PDGFs contain five immunoglobulin (Ig)-like domains, each ranging from about 88 to about 114 amino acids in length. See Lokker et al., J Biol. Chem., 1997, 272(52):33037-44, Miyazawa et al., J Biol. Chem., 1998, 273(39):25495-502; and Mahadevan et al., J Biol. Chem., 1995, 270(46):27595-600, which are incorporated herein by reference their entirety. The nature of the receptor binding specificities of VEGF-A and PDGF-B allow targeting to multiple forms of VEGFR and PDGFR (VEGFR1, VEGFR2, PDGFR-α, and PDGFR-β), respectively.

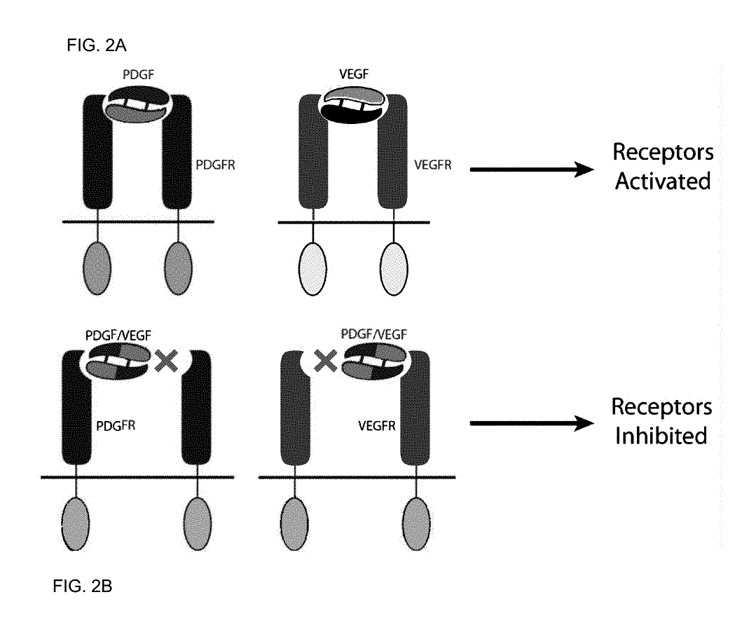

Hybrid Polypeptide. As used herein, the term “hybrid polypeptide” or “hybrid chimera polypeptide” refers to a polypeptide comprised of alternating sequences from two distinct parent proteins, in particular VEGF and PDGF. The breakpoints between patches may be determined by study of the relative protein structures. The resulting hybrid protein, when assembled, provides for binding at one pole to to PDGFR and at the other pole to VEGFR, shown schematically in FIG. 2B. Examples of hybrid proteins are provided here, e.g. in the sequence listing, SEQ ID NO: 3-25.

As with the native VEGF and PDGF proteins, the hybrid proteins of the invention may be produced as the individual chains, HC1 and HC2, or preferably as a fusion protein of the components, e.g. HC1-linker-HC2, or HC2-linker-HC1. In some embodiments an HC1 chain, i.e. one of SEQ ID NO: 8, 10, 12, 14, 16, 18, 20, 22 or 24 is fused through a linker to an HC2 chain independently selected from SEQ ID NO: 9, 11, 13, 15, 17, 19, 21, 23 or 25.

Amino acid sequence variants of hybrid proteins provided herein are also contemplated. For example, binding affinity and/or other biological properties can be improved by altering the amino acid sequence encoding the protein. Amino acids sequence variants can be prepared by introducing appropriate modifications into the nucleic acid sequence encoding the protein or by introducing the modification by peptide synthesis. Such modifications include, for example, deletions from, insertions into, and/or substitutions within the amino acid sequence. Any combination of deletion, insertion, and substitution can be made to arrive at the final amino acid construct, provided that the final construct possesses the desired characteristics such as binding to VEGFR or PDGFR. Accordingly, provided herein are variants of a hybrid protein, e.g. one or both of an HC1 or HC2 polypeptide disclosed herein. In some embodiments, variants comprise an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the amino acid sequence of any one of the provided hybrid polypeptides, where, for example, the variant may include 1, 2, 3, 4, 5, 6,7, 8, 9, 10 amino acid substitutions or changes relative to the provided sequence.

Modifications and changes can be made in the structure of the polypeptides and proteins of this disclosure and still result in a molecule having similar characteristics as the polypeptide (e.g., a conservative amino acid substitution). For example, certain amino acids can be substituted for other amino acids in a sequence without appreciable loss of activity. Because it is the interactive capacity and nature of a polypeptide that defines that polypeptide's or protein's biological functional activity, certain amino acid sequence substitutions can be made in a polypeptide or protein sequence and nevertheless obtain a polypeptide or protein with like properties.

Amino acid substitutions are generally based on the relative similarity of the amino acid side-chain substituents, for example, their hydrophobicity, hydrophilicity, charge, size, and the like. Exemplary substitutions that take one or more of the foregoing characteristics into consideration are well known to those of skill in the art and include, but are not limited to (original residue: exemplary substitution): (Ala: Gly, Ser), (Arg: Lys), (Asn: Gln, His), (Asp: Glu, Cys, Ser), (Gln: Asn), (Glu: Asp), (Gly: Ala), (His: Asn, Gln), (Ile: Leu, Val), (Leu: Ile, Val), (Lys: Arg), (Met: Leu, Tyr), (Ser: Thr), (Thr: Ser), (Tip: Tyr), (Tyr: Trp, Phe), and (Val: Ile, Leu). Embodiments of this disclosure, therefore, consider functional or biological equivalents of a polypeptide or protein as set forth above. In particular, embodiments of the polypeptides and proteins can include variants having about 50%, 60%, 70%, 80%, 90%, and 95% sequence identity to the polypeptide and protein of interest.

“Identity,” as known in the art, is a relationship between two or more polypeptide or protein sequences, as determined by comparing the sequences. In the art, “identity” also refers to the degree of sequence relatedness between polypeptides or proteins, as determined by the match between strings of such sequences. “Identity” can be readily calculated by known bioinformational methods.

Amino acid substitutions in the VEGF- and/or PDGF-derived sequences that alter the affinity and/or stability of a polypeptide for VEGFR or PDGFR are of interest for inclusion with the hybrid polypeptides of the invention. Such amino acid changes include, without limitation, those listed below. The amino acid residue numbering is made relative to the reference PDGF and VEGF sequences set forth in SEQ ID NO: 1 and SEQ ID NO: 2, which can be used in either chain of the hybrid.

TABLE 1

| Substitutions in VEGF sequence that | Substitutions in PDGF sequence that |
|---|---|
| enhance VEGFR binding | enhance VEGFR binding |
| V14A, V14I, V15A, K16R, F17L, M18R, | A8V, M12V, T18A, I25V, R56G, N57S, Q59R, |
| D19G, Q22R, R23K, I29F, I29V, L32S, I35V, | I77T, K80R, F84L, A87T, A96T K98I, T101A |
| F36L, F36S, D41N, E42K, E44G, Y45H, | |
| F47I, F47S, K48E, P49L, S50P, P53S, | |
| G58S, C60Y, D63H, D63N, D63G, E67G, | |
| I76T, M78V, Q79H, I80V, M81T, M81V, | |
| R82G, H86Y, Q87R, Q89H, H90R, I91T, | |
| I91V, G92D, S95T, N100D, K101E, E103V, | |
| K107R, D109V | |
| Substitutions in VEGF sequence that | Substitutions in PDGF sequence that |
| enhance PDGFR binding | enhance PDGFR binding |
| K16R, Y21H, I29M, I29V, L32S, D41N, | E9G, I13F, R28G, I30V, V39A, V72A, I77F, |
| F47S, V69A, M81I, K84T, I91F, L97F, | K80R, L95S, K98R |
| D109V | |
| Substitutions in VEGF sequence that | Substitutions in PDGF sequence that |
| enhance VEGFR and/or PDGFR binding | enhance VEGFR and/or PDGFR binding |
| T71A, Q79R, I83V | R56K, T63A, T88S, E100V, E100R |
| | Substitutions in PDGF sequence that |
| | increase protein stability |
| | R32A |

A “vector,” as used herein, refers to a recombinant plasmid or virus that comprises a nucleic acid to be delivered into a host cell, either in vitro or in vivo.

The term “polynucleotide” or “nucleic acid” as used herein refers to a polymeric form of nucleotides of any length, either ribonucleotides or deoxyribonucleotides. Thus, this term includes, but is not limited to, single-, double- or multi-stranded DNA or RNA, genomic DNA, cDNA, DNA-RNA hybrids, or a polymer comprising purine and pyrimidine bases, or other natural, chemically or biochemically modified, non-natural, or derivatized nucleotide bases. The backbone of the polynucleotide can comprise sugars and phosphate groups (as may typically be found in RNA or DNA), or modified or substituted sugar or phosphate groups. Alternatively, the backbone of the polynucleotide can comprise a polymer of synthetic subunits such as phosphoramidates and thus can be a oligodeoxynucleoside phosphoramidate (P—NH₂) or a mixed phosphoramidate-phosphodiester oligomer. In addition, a double-stranded polynucleotide can be obtained from the single stranded polynucleotide product of chemical synthesis either by synthesizing the complementary strand and annealing the strands under appropriate conditions, or by synthesizing the complementary strand de novo using a DNA polymerase with an appropriate primer.

“Percent (%) sequence identity” with respect to a reference polypeptide or nucleic acid sequence is defined as the percentage of amino acid residues or nucleotides in a candidate sequence that are identical with the amino acid residues or nucleotides in the reference polypeptide or nucleic acid sequence, after aligning the sequences and introducing gaps, if necessary, to achieve the maximum percent sequence identity, and not considering any conservative substitutions as part of the sequence identity. Alignment for purposes of determining percent amino acid or nucleic acid sequence identity can be achieved in various ways that are within the skill in the art, for instance, using publicly available computer software programs, for example, those described in Current Protocols in Molecular Biology (Ausubel et al., eds., 1987), Supp. 30, section 7.7.18, Table 7.7.1, and including BLAST, BLAST-2, ALIGN or Megalign (DNASTAR) software. A preferred alignment program is ALIGN Plus (Scientific and Educational Software, Pennsylvania). Those skilled in the art can determine appropriate parameters for measuring alignment, including any algorithms needed to achieve maximal alignment over the full length of the sequences being compared. For purposes herein, the amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows: 100 times the fraction X/Y, where X is the number of amino acid residues scored as identical matches by the sequence alignment program in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. For purposes herein, the % nucleic acid sequence identity of a given nucleic acid sequence C to, with, or against a given nucleic acid sequence D (which can alternatively be phrased as a given nucleic acid sequence C that has or comprises a certain % nucleic acid sequence identity to, with, or against a given nucleic acid sequence D) is calculated as follows: 100 times the fraction W/Z, where W is the number of nucleotides scored as identical matches by the sequence alignment program in that program's alignment of C and D, and where Z is the total number of nucleotides in D. It will be appreciated that where the length of nucleic acid sequence C is not equal to the length of nucleic acid sequence D, the % nucleic acid sequence identity of C to D will not equal the % nucleic acid sequence identity of D to C.

An “isolated” molecule (e.g., nucleic acid or protein) or cell means it has been identified and separated and/or recovered from a component of its natural environment.

An “effective amount” is an amount sufficient to effect beneficial or desired results, including clinical results. An effective amount can be administered in one or more administrations. In terms of a disease state, an effective amount is an amount sufficient to ameliorate, stabilize, or delay development of a disease.

An “individual” or “subject” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain embodiments, the individual or subject is a human.

As used herein, “treatment” is an approach for obtaining beneficial or desired clinical results. For purposes of this invention, beneficial or desired clinical results include, but are not limited to, alleviation of symptoms, diminishment of extent of disease, stabilized (i.e., not worsening) state of disease, preventing spread (i.e., metastasis) of disease, delay or slowing of disease progression, amelioration or palliation of the disease state, and remission (whether partial or total), whether detectable or undetectable. “Treatment” can also mean prolonging survival as compared to expected survival if not receiving treatment.

Reference to “about” a value or parameter herein includes (and describes) embodiments that are directed to that value or parameter per se. For example, description referring to “about X” includes description of “X.”

As used herein, the singular form of the articles “a,” “an,” and “the” includes plural references unless indicated otherwise.

It is understood that aspects and embodiments of the invention described herein include “comprising,” “consisting,” and/or “consisting essentially of” aspects and embodiments.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are herein described.

### Polypeptide Compositions

The hybrid polypeptides of the invention comprise two fused polypeptide chains, which polypeptide chains are a hybrid of alternating VEGF and PDGF sequences. Since VEGFR and PDGFR belong to the class of oligomeric cellular receptors that depend on oligomerization and/or conformational changes to be activated, binding of the hybrid polypeptides of the present invention without activation of the receptors allows the hybrid polypeptides to function as effective antagonists of VEGF- and PDGF-mediated phosphorylation and downstream signaling, which would otherwise result in growth and subsequent stabilization of neovasculature.

The hybrid polypeptide comprises two engineered polypeptide chains, which may be referred to herein as hybrid chain 1 (HC1) and hybrid chain 2 (HC2), fused through a linker. The amino acid sequences may be discussed with reference to a wild-type human PDGF-B polypeptide, provided herein as SEQ ID NO: 1; and a wild-type human VEGF-A polypeptide, provided herein as SEQ ID NO: 2. Residues 7-104 from mature PDGF-B and residues 13-109 from mature VEGF-A were used in the construction of the polypeptide compositions of the invention. It will be understood by one of skill in the art that the naming is arbitrary and that the orientation of the two chains may be reversed, i.e. the polypeptide may be organized as HC1-linker-HC2; or as HC2-linker-HC1.

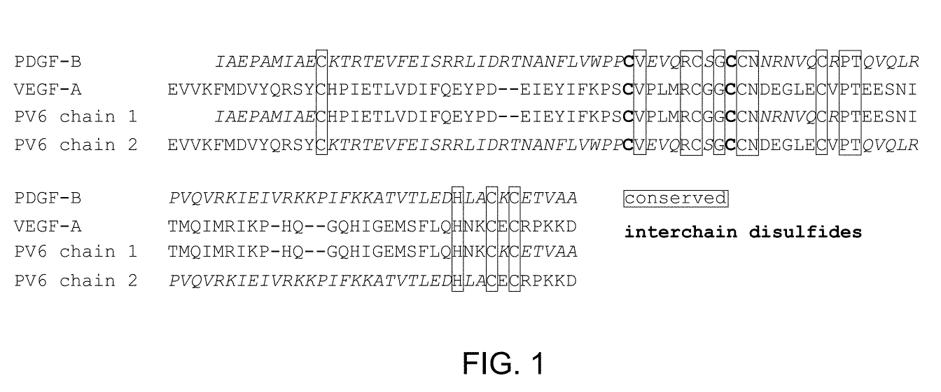

The hybrid structure (numbering described below with reference to SEQ ID NO: 1,2 and the sequence alignment shown in FIG. 1) alternates blocks of sequence from PDGF and from VEGF. The blocks of sequence are aligned and can be modified to maintain the overall length of the polypeptide, for example where the reference wild-type PDGF sequence is 98 amino acids in length and VEGF sequence is 97 in length, the hybrid sequence may be from about 90-105 amino acids in length. The spacing and presence of conserved residues is retained, such as in HC1 (relative to SEQ ID NO: 8), which contains cysteine at positions 10, 35, 41, 44, 45, 52, 86 and 88. In HC2 (relative to SEQ ID NO: 9) cysteine residues may be at positions 14, 41, 47, 50, 51, 58, 95 and 97. Conserved amino acids are common to the aligned VEGF and PDGF sequences, and thus can be attributed to either parental sequence. SEQ ID NO: 3 is a non-limiting example of a full length hybrid polypeptide of the invention.

The HC1 chain comprises 5 alternating blocks, arranged as PDGF-B/VEGF-A/PDGF-B/VEGF-A/PDGF-B. In some embodiments the first block (B1a) comprises from about 10-13 residues of PDGF-B, starting from residue 7 of the mature protein up to residue 16-19 and including the cysteine at residue 16 (referring to SEQ ID NO: 1). The second block of sequence (B1b) comprises from about 28-37 amino acid residues of VEGF-A, starting from residue 26-30 up to residue 57-62, including arginine 56 and cysteine 57 (referring to SEQ ID NO: 2). The third block (B1c) comprises from about 9-16 amino acid residues of PDGF-B, starting from residue 48-55 up to residue 63, including proline 62 and threonine 63. The fourth block (Bid) comprises from about 28-33 amino acid residues of VEGF-A, starting from residue 70-72 up to 99-102, including histidine 99. The final block of sequence (B1e) comprises from about 7-11 amino acid residues of PDGF-B, starting from residue 94-98 up to residue 104. The blocks are shown in the Table below:

TABLE 2A

| | Sequence | Start | | Length | |
|---|---|---|---|---|---|
| Block | identification | aa | End aa | (aa) | exemplary sequence |
| B1a | PDGF | 7 | 16-19 | 10-13 | IAEPAMIAEC(KTR) |
| B1b | VEGF | 26-30 | 57-62 | 28-37 | (CHPI)ETLVDIFQEYPDEIEYIFKPSCVP |
| | | | | | LMRC(GGCCN) |
| B1c | PDGF | 48-55 | 63 | 9-16 | (RCSGCCN)NRNVQCRPT |
| B1d | VEGF | 70-72 | 99-102 | 28-33 | (PT)EESNITMQIMRIKPHQGQHIGEMS |
| | | | | | FLQH(NKC) |
| B1e | PDGF | 94-98 | 104 | 7-11 | (HLAC)KCETVAA |

In other embodiments, HC1 is described as follows: the first block (B1a) comprises from about 10-13 residues of PDGF-B, starting from residue 7 of the mature protein up to residue 16-19 and including the cysteine at residue 16. The second block of sequence (B1b) comprises from about 23-37 amino acid residues of VEGF-A, starting from residue 26-30 up to residue 52-62, including the cysteine at residue 51 and valine at residue 52 (numbering with reference to SEQ ID NO: 2). The third block (Bic) comprises from about 6-21 amino acid residues of PDGF-B, starting from residue 43-55 up to residue 60-63, including cysteine 60. The fourth block (B1d) comprises from about 28-37 amino acid residues of VEGF-A, starting from residue 68-72 up to 99-104, including histidine 99. The final block of sequence (B1e) comprises from about 5-11 amino acid residues of PDGF-B, starting from residue 94-100 up to residue 104. The blocks are shown in the table below:

TABLE 2B

| | Sequence | Start | | Length | |
|---|---|---|---|---|---|
| Block | identification | aa | End aa | (aa) | exemplary sequence |
| B1a | PDGF | 7 | 16-19 | 10-13 | IAEPAMIAEC(KTR) |
| B1b | VEGF | 26-30 | 52-62 | 23-37 | (CHPI)ETLVDIFQEYPDEIEYIFKPSCV |
| | | | | | (PLMRCGGCCN) |
| B1c | PDGF | 43-55 | 60-63 | 6-21 | (CVEVQRCSGCCN)NRNVQC(RPT) |
| B1d | VEGF | 68-72 | 99-104 | 28-37 | (CVPT)EESNITMQIMRIKPHQGQHIGE |
| | | | | | MSFLQH(NKCEC) |
| B1e | PDGF | 94-100 | 104 | 5-11 | (HLACKC)ETVAA |

Non-limiting examples of chain 1 polypeptides include SEQ ID NO: 3, residues 1-93; and SEQ ID NO: 8, 10, 12, 14, 16, 18, 20, 22 and 24. With reference to SEQ ID NO: 8, amino acid changes that enhance affinity for PDGFR include, without limitation, K87R. This corresponds to K98R in PDGF (referring to SEQ ID NO: 1). With reference to SEQ ID NO: 8, amino acid changes that enhance affinity for VEGFR include, without limitation, one or more of M6V, F2OL, E28G, and Q71R. In some embodiments, the amino acid changes are selected from F2OL, E28G, and Q71R. These substitutions correspond to M12V in PDGF (referring to SEQ ID NO: 1); and F36L, E44G, and Q87R in VEGF (referring to SEQ ID NO: 2).

The HC2 chain comprises 5 alternating blocks, arranged as VEGF-A/PDGF-B/VEGF-A/PDGF-B/VEGF-A. The first block (B2a) comprises from about 14-17 residues of VEGF-A, starting from residue 13 of the mature protein up to residue 26-29 and including the cysteine at residue 26 (referring to SEQ ID NO: 2). The second block of sequence (B2b) comprises from about 30-39 amino acid residues of PDGF-B, starting from residue 16-20 up to residue 49-54 and including arginine 48 and cysteine 49 (referring to SEQ ID NO: 1). The third block (B2c) comprises from about 9-16 residues of VEGF-A, starting from residue 56-63 up to residue 71, including proline 70 and threonine 71. The fourth block (B2d) comprises from about 31-36 amino acid residues of PDGF-B, starting from residue 62-64 up to residue 94-97, including histidine 94. The final block of sequence (B2e) comprises from about 7-11 residues of VEGF, starting from residue 99-103 up to residue 109. The blocks are shown in the table below:

TABLE 3A

| | Sequence | Start | | Length | |
|---|---|---|---|---|---|
| Block | identification | aa | End aa | (aa) | exemplary sequence |
| B2a | VEGF | 13 | 26-29 | 14-17 | EVVKFMDVYQRSYC(HPI) |
| B2b | PDGF | 16-20 | 49-54 | 30-39 | (CKTR)TEVFEISRRLIDRTNANFLVWPP |
| | | | | | CVEVQRC(SGCCN) |
| B2c | VEGF | 56-63 | 71 | 9-16 | (RCGGCCN)DEGLECVPT |
| B2d | PDGF | 62-64 | 94-97 | 31-36 | (PT)QVQLRPVQVRKIEIVRKKPIFKKAT |
| | | | | | VTLEDH(LAC) |
| B2e | VEGF | 99-103 | 109 | 7-11 | (HNKC)ECRPKKD |

In other embodiments, HC2 is described as follows: the first block (B2a) comprises from about 14-17 residues of VEGF-A, starting from residue 13 of the mature protein up to residue 26-29 and including the cysteine at residue 26. The second block of sequence (B2b) comprises from about 25-39 amino acid residues of PDGF-B, starting from residue 16-20 up to residue 44-54 and including cysteine 43 and valine 44. The third block (B2c) comprises from about 6-21 residues of VEGF-A, starting from residue 51-63 up to residue 68-71, including cysteine 68. The fourth block (B2d) comprises from about 31-40 amino acid residues of PDGF-B, starting from residue 60-64 up to residue 94-99, including histidine 94. The final block of sequence (B2e) comprises from about 5-11 residues of VEGF, starting from residue 99-105 up to residue 109. The blocks are shown in the table below:

TABLE 3B

| | Sequence | Start | | Length | |
|---|---|---|---|---|---|
| Block | identification | aa | End aa | (aa) | exemplary sequence |
| B2a | VEGF | 13 | 26-29 | 14-17 | EVVKFMDVYQRSYC(HPI) |
| B2b | PDGF | 16-20 | 44-54 | 25-39 | (CKTR)TEVFEISRRLIDRTNANFLVWPP |
| | | | | | CV(EVQRCSGCCN) |
| B2c | VEGF | 51-63 | 68-71 | 6-21 | (CVPLMRCGGCCN)DEGLEC(VPT) |
| B2d | PDGF | 60-64 | 94-99 | 31-40 | (CRPT)QVQLRPVQVRKIEIVRKKPIFKK |
| | | | | | ATVTLEDH(LACKC) |
| B2e | VEGF | 99-105 | 109 | 5-11 | (HNKCEC)RPKKD |

Non-limiting examples of chain 2 polypeptides include SEQ ID NO: 3, residues 108-209; and SEQ ID NO: 9, 11, 13, 15, 17, 19, 21, 23 and 25. With reference to SEQ ID NO: 9, amino acid changes that enhance affinity for PDGFR include, without limitation, I75F. This corresponds to I77F in PDGF (referring to SEQ ID NO: 1). With reference to SEQ ID NO: 9, amino acid changes that enhance affinity for VEGFR include, without limitation, D53H and D102V. This corresponds to D63H and D109V in VEGF (referring to SEQ ID NO: 2). With reference to SEQ ID NO: 9, the amino acid change R30A removes a protease site and can improve stability. This corresponds to R32A in PDGF (referring to SEQ ID NO: 1), see Cook et al. (1992) Biochem J.

The hybrid polypeptides of the invention bind coordinately to VEGF and PDGF receptors but do not induce receptor activation, thereby simultaneously antagonizing VEGF- and PDGF-stimulated receptor autophosphorylation and proliferation of cells. Compositions include one or more hybrid polypeptide(s) of the invention, which may be provided as a single species or as a cocktail of two or more polypeptides, usually in combination with a pharmaceutically acceptable excipient.

The two hybrid polypeptide chains may be linked by a linking moiety such as a peptide linker. In some embodiments, the linker moiety is a peptide linker. In some embodiments, the peptide linker comprises 2 to 100 amino acids. In some embodiments, the peptide linker comprises 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 but no greater than 100 amino acids. In some embodiments, the peptide linker is between 5 to 75, 5 to 50, 5 to 25, 5 to 20, 5 to 15, 5 to 10 or 5 to 9 amino acids in length. Exemplary linkers include linear peptides having at least two amino acid residues such as Gly-Gly, Gly-Ala-Gly, Gly-Pro-Ala, Gly-Gly-Gly-Gly-Ser. Suitable linear peptides include poly glycine, polyserine, polyproline, polyalanine and oligopeptides consisting of alanyl and/or serinyl and/or prolinyl and/or glycyl amino acid residues. In some embodiments, the peptide linker comprises the amino acid sequence selected from the group consisting of Gly₉, Glu₉, Ser₉, Gly₅-Cys-Pro₂-Cys, (Gly₄-Ser)₃, Ser-Cys-Val-Pro-Leu-Met-Arg-Cys-Gly-Gly-Cys-Cys-Asn, Pro-Ser-Cys-Val-Pro-Leu-Met-Arg-Cys-Gly-Gly-Cys-Cys-Asn, Gly-Asp-Leu-Ile-Tyr-Arg-Asn-Gln-Lys, and Gly₉-Pro-Ser-Cys-Val-Pro-Leu-Met-Arg-Cys-Gly-Gly-Cys-Cys-Asn.

In one embodiment a linker comprises the amino acid sequence GSTSGSGKSSEGKG, or (GGGGS)n, where n is 1, 2, 3, 4, 5, etc.; however many such linkers are known and used in the art and may serve this purpose. The polypeptides of the invention are typically provided in single-chain form, which means that the HC1 and HC2 are linked by peptide bonds through a linker peptide, resulting in the formation of the desired heterodimer, rather than being linked exclusively by noncovalent bonds or disulfide bonds found in the native growth factor proteins.

In some such embodiments, a hybrid polypeptide of the invention is fused or otherwise joined to an immunoglobulin sequence to form a chimeric protein. The immunoglobulin sequence preferably, but not necessarily, is immunoglobulin constant domain(s). The immunoglobulin moiety in such chimeras may be obtained from any species, usually human, and includes IgG1, IgG2, IgG3 or IgG4 subtypes, IgA, IgE, IgD or IgM. The immunoglobulin moiety may comprise one or more domains, e.g. CH1, CH2, CH3, etc.

Chimeras constructed from a sequence linked to an appropriate immunoglobulin constant domain sequence are known in the art. In such fusions the encoded chimeric polypeptide may retain at least functionally active hinge, CH2 and CH3 domains of the constant region of an immunoglobulin heavy chain. Fusions are also made to the C-terminus of the Fc portion of a constant domain, or immediately N-terminal to the CH1 of the heavy chain or the corresponding region of the light chain. The precise site at which the fusion is made is not critical; particular sites are well known and may be selected in order to optimize the biological activity, secretion or binding characteristics of the hybrid polypeptide-immunoglobulin chimeras. In some embodiments, the chimeras are assembled as monomers, or hetero- or homo-multimers, and particularly as dimers or tetramers.

Although the presence of an immunoglobulin light chain is not required, an immunoglobulin light chain may be included, either covalently associated to an immunoglobulin heavy chain fusion polypeptide, or directly fused to the hybrid polypeptide. A single chain construct may be used to provide both heavy and light chain constant regions.

In other embodiments of the invention, a trap polypeptide is generated to provide antagonist activity. In such a polypeptide, one or more extracellular domains of a receptor are fused or otherwise joined to an immunoglobulin sequence to form a chimeric protein, where the immunoglobulin sequence can be one or more constant domains, as described above.

For example a PDGRβ-Fc fusion, including without limitation the polypeptide described in Example 5, comprises the first three Ig-like domains of PDGFR-β fused to an Fc sequence. The PDGFR sequences for use in such a polypeptide include wild-type and engineered sequences, including variant PDGFR receptors known in the art, or engineered for enhanced binding, altering the PDGFR isotypes binding specificity, stability, and other clinically relevant parameters including, but not limited to serum half-life. This polypeptide provides a ligand trap by binding to and neutralizing soluble PDGF ligand.

Polypeptides can be produced through recombinant methods and chemical synthesis. In addition, functionally equivalent polypeptides may find use, where the equivalent polypeptide may contain deletions, additions or substitutions of amino acid residues that result in a silent change, thus producing a functionally equivalent differentially expressed on pathway gene product. Amino acid substitutions may be made on the basis of similarity in polarity, charge, solubility, hydrophobicity, hydrophilicity, and/or the amphipathic nature of the residues involved. “Functionally equivalent”, as used herein, refers to a protein capable of exhibiting a substantially similar in vivo activity.

The polypeptides may be produced by recombinant DNA technology using techniques well known in the art. Methods which are well known to those skilled in the art can be used to construct expression vectors containing coding sequences and appropriate transcriptional/translational control signals. These methods include, for example, in vitro recombinant DNA techniques, synthetic techniques and in vivo recombination/genetic recombination. Alternatively, RNA capable of encoding the polypeptides of interest may be chemically synthesized.

As an option to recombinant methods, polypeptides can be chemically synthesized. Such methods typically include solid-state approaches, but can also utilize solution based chemistries and combinations or combinations of solid-state and solution approaches. Examples of solid-state methodologies for synthesizing proteins are described by Merrifield (1964) J. Am. Chem. Soc. 85:2149; and Houghton (1985) Proc. Natl. Acad. Sci., 82:5132. Fragments of polypeptides of the invention protein can be synthesized and then joined together. Methods for conducting such reactions are described by Grant (1992) Synthetic Peptides: A User Guide, W.H. Freeman and Co., N.Y.; and in “Principles of Peptide Synthesis,” (Bodansky and Trost, ed.), Springer-Verlag, Inc. N.Y., (1993). Proteins or peptides of the invention may comprise one or more non-naturally occurring or modified amino acids. A “non-naturally occurring amino acid residue” refers to a residue, other than those naturally occurring amino acid residues listed above, which is able to covalently bind adjacent amino acid residues(s) in a polypeptide chain. Non-natural amino acids include, but are not limited to homo-lysine, homo-arginine, homo-serine, azetidinecarboxylic acid, 2-aminoadipic acid, 3-aminoadipic acid, beta-alanine, aminopropionic acid, 2-aminobutyric acid, 4-aminobutyric acid, 6-aminocaproic acid, 2-aminoheptanoic acid, 2-aminoisobutyric acid, 3-aminoisbutyric acid, 2-aminopimelic acid, tertiary-butylglycine, 2,4-diaminoisobutyric acid, desmosine, 2,2′-diaminopimelic acid, 2,3-diaminopropionic acid, N-ethylglycine, N-ethylasparagine, homoproline, hydroxylysine, allo-hydroxylysine, 3-hydroxyproline, 4-hydroxyproline, isodesmosine, allo-isoleucine, N-methylalanine, N-methylglycine, N-methylisoleucine, N-methylpentylglycine, N-methylvaline, naphthalanine, norvaline, norleucine, ornithine, citrulline, pentylglycine, pipecolic acid and thioproline. Modified amino acids include natural and non-natural amino acids which are chemically blocked, reversibly or irreversibly, or modified on their N-terminal amino group or their side chain groups, as for example, N-methylated D and L amino acids, side chain functional groups that are chemically modified to another functional group. For example, modified amino acids include methionine sulfoxide; methionine sulfone; aspartic acid- (beta-methyl ester), a modified amino acid of aspartic acid; N-ethylglycine, a modified amino acid of glycine; or alanine carboxamide and a modified amino acid of alanine. Additional non-natural and modified amino acids, and methods of incorporating them into proteins and peptides, are known in the art (see, e.g., Sandberg et al., (1998) J. Med. Chem. 41: 2481-91; Xie and Schultz (2005) Curr. Opin. Chem. Biol. 9: 548-554; Hodgson and Sanderson (2004) Chem. Soc. Rev. 33: 422-430.

Amino acid sequence insertions include amino- (“N”) and/or carboxy- (“C”) terminal hybrids ranging in length from one residue to a hundred or more residues, as well as intrasequence insertions of single or multiple amino acid residues. Examples of terminal insertions include a hybrid protein with an N-terminal methionyl residue or the hybrid protein fused to a cytotoxic polypeptide. Other insertional variants of the hybrid protein molecule include N- or C-terminus fusion of the hybrid protein a polypeptide that allows formation of protein multimers; a polypeptide tag for purification; and the like as known in the art.

In some embodiments, a hybrid polypeptide is modified to have an increased plasma and/or ocular half-life as compared to a wild-type VEGF or PDGF protein, or relative to a hybrid protein of the invention prior to the modification. The half-life of a protein is a measurement of protein stability and its rate of clearance and indicates the time necessary for a one-half reduction in the concentration of the protein. In some embodiments, the serum half-life of the hybrid molecules described herein is determined by any suitable method for measuring protein levels in samples from a subject over time, such as immunoassays using antibodies to measure protein levels in serum samples taken over a period of time after administration of the hybrid, or by detection of labeled hybrid molecules, e.g., radiolabeled molecules, in samples taken from a subject after administration of the labeled hybrid.

Any suitable modification is used to increase the half-life of a hybrid polypeptide disclosed herein. In some embodiments, increased half-life is provided by the use of a Fc-fusion. In some embodiments, increased half-life is provided by the use of a peptide extension such as a carboxy terminal extension peptide (CTEP) of human chorionic gonadotropin (hCG). In some embodiments, a monomer of a hybrid is covalently bound to a CTEP, e.g. by a peptide bond or by a heterobifunctional reagent able to form a covalent bond between the amino terminus and carboxyl terminus of a protein, including but not limited to a peptide linker. In some embodiments, a hybrid comprises an amino acid substitution coupled with one or more amino acid substitutions that enhance stability and increase serum half-life by eliminating one or more proteolytic cleavage sites. In some embodiments, the additional amino acid substitutions reduce proteolytic cleavage. In some embodiments, the additional amino acid substitutions prevent proteolytic cleavage. In some embodiments, increased half-life is provided by crosslinking, including but not limited to pegylation or conjugation of other appropriate chemical groups. In some embodiments, half-life is increased by increasing the number of negatively charged residues within the molecule, for instance, the number of glutamate and/or aspartate residues. In some embodiments, such alteration is accomplished by site directed mutagenesis or by an insertion of an amino acid sequence containing one or more negatively charged residues.

Typically, the coding sequence is placed under the control of a promoter that is functional in the desired host cell to produce relatively large quantities of the gene product. An extremely wide variety of promoters are well-known, and can be used in the expression vectors of the invention, depending on the particular application. Ordinarily, the promoter selected depends upon the cell in which the promoter is to be active. Other expression control sequences such as ribosome binding sites, transcription termination sites and the like are also optionally included. Constructs that include one or more of these control sequences are termed “expression cassettes.” Expression can be achieved in prokaryotic and eukaryotic cells utilizing promoters and other regulatory agents appropriate for the particular host cell. Exemplary host cells include, but are not limited to, E. coli, other bacterial hosts, yeast, and various higher eukaryotic cells such as the COS, CHO and HeLa cells lines and myeloma cell lines.

Proteins may be purified and identified using commonly known methods such as fractionation on immunoaffinity or ion-exchange columns; ethanol precipitation; reverse phase HPLC; chromatography on silica or on a cation exchange resin such as DEAE; chromatofocusing; SDS-PAGE; ammonium sulfate precipitation; gel filtration using, for example, Sephadex G-75; hydrophobic affinity resins, ligand affinity using a suitable binding partner immobilized on a matrix, centrifugation, ELISA, BIACore, Western blot assay, amino acid and nucleic acid sequencing, and biological activity.

The hybrid proteins or hybrid protein components disclosed herein may be characterized or assessed for biological activities including, but not limited to, affinity to a target binding partner (e.g., a PDGFR and/or VEGFR family protein), competitive binding, inhibitory activity (e.g., inhibition of PDGF or VEGF pathway activation), inhibition of cell proliferation, inhibition of tumor growth, and inhibition of angiogenesis (e.g., choroidal neovascularization). In some embodiments, the hybrid proteins or hybrid protein components disclosed herein can be assessed for biological activity in vivo or in vitro.

The hybrid proteins or hybrid protein components disclosed herein can be assessed for affinity to a binding partner such as a PDGFR family protein and/or a VEGFR family protein. Many methods for assessing binding affinity are known in the art and can be used to identify the binding affinities of hybrid proteins or hybrid protein components to a binding partner. Binding affinities can be expressed as dissociation constant (Kd) values or half maximal effective concentration (EC50) values. Techniques for determining binding affinities (e.g., Kd values) are well known in the art such as Enzyme-Linked Immunosorbent Assay (ELISA) and BlAcore. See Harlow and Lane, Antibodies: A Laboratory Manual, CSH Publications, NY (1988); Ausubel et al., Current Protocols in Molecular Biology, John Wiley & Sons, New York, (2009); Altschuh et al., Biochem., 31:6298 (1992); and the BlAcore method disclosed by Pharmacia Biosensor, all of which are incorporated herein by reference. For example, binding affinities of the hybrid proteins to a binding partner can be determined using ELISA.

In any of the embodiments herein, a hybrid protein has an IC50 of ≤1 μM, ≤100 nM, ≤10 nM, ≤1 nM, ≤0.1 nM, ≤0.01 nM, or ≤0.001 nM (e.g., 10⁻⁸M or less, e.g., from 10⁻⁸M to 10⁻¹³M, e.g., from 10⁻⁹M to 10⁻¹³ M) for inhibition of an activity (e.g., inhibition of PDGFR activity and/or VEGFR activity). In any of the embodiments herein, a hybrid protein has a Kd for a binding partner (e.g., PDGFR and/or VEGFR) of less than about any of about 1.0 mM, 500 μM, 100 μM, 50 μM, 25 μM, 10 μM, 5 μM, 1 μM, 900 nM, 800 nM, 700 nM, 600 nM, 500 nM, 400 nM, 350 nM, 300 nM, 250 nM, 200 nM, 150 nM, 100 nM, 95 nM, 90 nM, 85 nM, 80 nM, 75 nM, 70 nM, 65 nM, 60 nM, 55 nM, 50 nM, 45 nM, 40 nM, 35 nM, 30 nM, 25 nM, 20 nM, 15 nM, 10 nM, 5 nM, 1 nM, 900 pM, 800 pM, 700 pM, 600 pM, 500 pM, 400 pM, 300 pM, 200 pM, 100 pM, 50 pM, 25 pM, 12.5 pM, 6.25 pM, 5 pM, 4 pM, or 3 pM, inclusive, including any values in between these numbers.

In some embodiments, the hybrid proteins disclosed herein can be assessed for anti-proliferative activities such as reduction of cell proliferation. Many methods for assessing anti-proliferative properties for a hybrid protein are known in the art. In one exemplary assay, human umbilical vein endothelial cells (HUVECs) can be used to demonstrate inhibition of VEGF-dependent cell proliferation by a hybrid protein described herein, and human fibroblasts BJ-5ta and mouse fibroblasts NIH/3T3 for PDGF-dependent cell proliferation. In this assay, the hybrid protein is applied to HUVECs, BJ-5ta, or NIH/3T3 in the presence of VEGF and/or PDGF and cell proliferation is measured.

In some embodiments, anti-angiogenic properties for a hybrid protein are measured using techniques known in the art. In an exemplary assay, an animal model of wet age-related macular degeneration is used to assay inhibition of neovascularization in the eye by the hybrid protein.

The polypeptide may be labeled, either directly or indirectly. Any of a variety of suitable labeling systems may be used, including but not limited to, radioisotopes such as ¹²⁵1; enzyme labeling systems that generate a detectable colorimetric signal or light when exposed to substrate; and fluorescent labels. Indirect labeling involves the use of a protein, such as a labeled antibody, that specifically binds to the polypeptide of interest. Such antibodies include but are not limited to polyclonal, monoclonal, chimeric, single chain, Fab fragments and fragments produced by a Fab expression library. Other polypeptide tags of interest include birA, sortase, etc. as known in the art.

The polypeptides of the invention can be coupled or conjugated to one or more cytotoxic or imaging moieties. As used herein, “cytotoxic moiety” is a moiety that inhibits cell growth or promotes cell death when proximate to or absorbed by the cell. Suitable cytotoxic moieties in this regard include radioactive isotopes (radionuclides), chemotoxic agents such as differentiation inducers and small chemotoxic drugs, toxin proteins, and derivatives thereof. “Imaging moiety” (I) is a moiety that can be utilized to increase contrast between a tumor and the surrounding healthy tissue in a visualization technique (e.g., radiography, positron-emission tomography, single-photon emission computed tomography, near-infrared fluorescence imaging, magnetic resonance imaging, ultrasound, direct or indirect visual inspection). Thus, suitable imaging moieties include radiography moieties (e.g. heavy metals and radiation emitting moieties), positron emitting moieties, magnetic resonance contrast moieties, gas-filled mirobubble spheres for contrast-enhanced ultrasound, and optically visible moieties (e.g., fluorescent or visible-spectrum dyes, visible particles, etc.). It will be appreciated by one of ordinary skill that some overlap exists between therapeutic and imaging moieties. For instance ²¹²Pb and ²¹²Bi are both useful radioisotopes for therapeutic compositions, but are also electron-dense, and thus provide contrast for X-ray radiographic imaging techniques, and can also be utilized in scintillation imaging techniques.

In general, therapeutic or imaging agents may be conjugated to the polypeptides of the invention by any suitable technique, with appropriate consideration of the need for pharmokinetic stability and reduced overall toxicity to the patient. A therapeutic agent may be coupled to a polypeptide either directly or indirectly (e.g. via a linker group). A direct reaction between an agent and a polypeptide is possible when each possesses a functional group capable of reacting with the other. For example, a nucleophilic group, such as an amino or sulfhydryl group, may be capable of reacting with a carbonyl-containing group, such as an anhydride or an acid halide, or with an alkyl group containing a good leaving group (e.g., a halide). Alternatively, a suitable chemical linker group may be used. A linker group can function as a spacer to distance a polypeptide from an agent in order to avoid interference with binding capabilities. A linker group can also serve to increase the chemical reactivity of a substituent on a moiety or a polypeptide, and thus increase the coupling efficiency. An increase in chemical reactivity may also facilitate the use of moieties, or functional groups on moieties, which otherwise would not be possible.

Suitable linkage chemistries include maleimidyl linkers and alkyl halide linkers (which react with a sulfhydryl on the polypeptide moiety) and succinimidyl linkers (which react with a primary amine on the polypeptide moiety). Several primary amine and sulfhydryl groups are present on a polypeptide, and additional groups may be designed into recombinant molecules. It will be evident to those skilled in the art that a variety of bifunctional or polyfunctional reagents, both homo- and hetero-functional (such as those described in the catalog of the Pierce Chemical Co., Rockford, Il.), may be employed as a linker group. Coupling may be effected, for example, through amino groups, carboxyl groups, sulfhydryl groups or oxidized carbohydrate residues. There are numerous references describing such methodology, e.g., U.S. Pat. No. 4,671,958. As an alternative coupling method, cytotoxic or imaging moieties may be coupled to the polypeptides of the invention through an oxidized carbohydrate group at a glycosylation site, as described in U.S. Pat. No. 5,057,313 and 5,156,840. Yet another alternative method of coupling a polypeptide to the cytotoxic or imaging moiety is by the use of a non-covalent binding pair, such as streptavidin/biotin, or avidin/biotin. In these embodiments, one member of the pair is covalently coupled to a polypeptide and the other member of the binding pair is covalently coupled to the cytotoxic or imaging moiety.

Carriers and linkers specific for radionuclide agents (both for use as cytotoxic moieties or positron-emission imaging moieties) include radiohalogenated small molecules and chelating compounds. For example, U.S. Pat. No. 4,735,792 discloses representative radiohalogenated small molecules and their synthesis. A radionuclide chelate may be formed from chelating compounds that include those containing nitrogen and sulfur atoms as the donor atoms for binding the metal, or metal oxide, radionuclide. Such chelation carriers are also useful for magnetic spin contrast ions for use in magnetic resonance imaging tumor visualization methods, and for the chelation of heavy metal ions for use in radiographic visualization methods.

Preferred radionuclides for use as cytotoxic moieties are radionuclides that are suitable for pharmacological administration. Such radionuclides include ¹²³I, ¹²⁵I, ¹³¹I, ⁹⁰Y, ²¹¹At, ⁶⁷Cu, ¹⁸⁶Re, ¹⁸⁸Re, ²¹²Pb, and ²¹²Bi. Iodine and astatine isotopes are more preferred radionuclides for use in the therapeutic compositions of the present invention, as a large body of literature has been accumulated regarding their use. ¹³¹I is particularly preferred, as are other β-radiation emitting nuclides, which have an effective range of several millimeters. ¹²³I, ¹²⁵I, ¹³¹I, or ²¹¹At may be conjugated to polypeptides of the invention for use in the compositions and methods utilizing any of several known conjugation reagents, including lodogen, N-succinimidyl 3-[²¹¹At]astatobenzoate, N-succinimidyl 3-[¹³¹I]iodobenzoate (SIB), and N-succinimidyl 5-[¹³¹I]iodob-3-pyridinecarboxylate (SIPC). Any iodine isotope may be utilized in the recited iodo-reagents. Radionuclides can be conjugated to polypeptides of the invention by suitable chelation agents known to those of skill in the nuclear medicine arts.

Preferred radiographic moieties for use as imaging moieties in the present invention include compounds and chelates with relatively large atoms, such as gold, iridium, technetium, barium, thallium, iodine, and their isotopes. It is preferred that less toxic radiographic imaging moieties, such as iodine or iodine isotopes, be utilized in the compositions and methods of the invention. Examples of such compositions, which may be utilized for x-ray radiography are described in U.S. Pat. No. 5,709,846, incorporated fully herein by reference. Such moieties may be conjugated to the polypeptides of the invention through an acceptable chemical linker or chelation carrier. In addition, radionuclides which emit radiation capable of penetrating the skull may be useful for scintillation imaging techniques. Suitable radionuclides for conjugation include ⁹⁹Tc, ¹¹¹In, and ⁶⁷Ga. Positron emitting moieties for use in the present invention include ¹⁸F, which can be easily conjugated by a fluorination reaction with the polypeptides of the invention according to the method described in U.S. Patent No. 6,187,284, or ⁶⁴Cu, which can be conjugated through chemical chelators as extensively described in the literature.

Preferred magnetic resonance contrast moieties include chelates of chromium(III), manganese(II), iron(II), nickel(II), copper(II), praseodymium(III), neodymium(III), samarium(III) and ytterbium(III) ion. Because of their very strong magnetic moment, the gadolinium(III), terbium(III), dysprosium(III), holmium(III), erbium(III), and iron(III) ions are especially preferred. Examples of such chelates, suitable for magnetic resonance spin imaging, are described in U.S. Pat. No. 5,733,522, incorporated fully herein by reference. Nuclear spin contrast chelates may be conjugated to the polypeptides of the invention through a suitable chemical linker.

Optically visible moieties for use as imaging moieties include fluorescent dyes, or visible-spectrum dyes, visible particles, and other visible labeling moieties. Fluorescent dyes such as ALEXA dyes, fluorescein, coumarin, rhodamine, bodipy Texas red, and cyanine dyes, are useful when sufficient excitation energy can be provided to the site to be inspected visually. Endoscopic visualization procedures may be more compatible with the use of such labels. For many procedures where imaging agents are useful, such as during an operation to resect a brain tumor, visible spectrum dyes are preferred. Acceptable dyes include FDA-approved food dyes and colors, which are non-toxic, although pharmaceutically acceptable dyes which have been approved for internal administration are preferred. In preferred embodiments, such dyes are encapsulated in carrier moieties, which are in turn conjugated to the polypeptides of the invention. Alternatively, visible particles, such as colloidal gold particles or latex particles, may be coupled to the polypeptides of the invention via a suitable chemical linker.

### Pharmaceutical Formulations

Formulations of polypeptides of the invention find use in diagnosis and therapy. The formulation may comprise one, two or more polypeptides of the invention. The therapeutic formulation may be administered in combination with other methods of treatment, e.g. chemotherapy, radiation therapy, surgery, and the like.

Formulations may be optimized for retention and stabilization at a targeted site. Stabilization techniques include enhancing the size of the polypeptide, by cross-linking, multimerizing, or linking to groups such as polyethylene glycol, polyacrylamide, neutral protein carriers, etc. in order to achieve an increase in molecular weight. Other strategies for increasing retention include the entrapment of the polypeptide in a biodegradable or bioerodible implant. The rate of release of the therapeutically active agent is controlled by the rate of transport through the polymeric matrix, and the biodegradation of the implant. The transport of polypeptide through the polymer barrier will also be affected by compound solubility, polymer hydrophilicity, extent of polymer cross-linking, expansion of the polymer upon water absorption so as to make the polymer barrier more permeable to the drug, geometry of the implant, and the like. The implants are of dimensions commensurate with the size and shape of the region selected as the site of implantation. Implants may be particles, sheets, patches, plaques, fibers, microcapsules and the like and may be of any size or shape compatible with the selected site of insertion.

Pharmaceutical compositions can include, depending on the formulation desired, pharmaceutically-acceptable, non-toxic carriers of diluents, which are defined as vehicles commonly used to formulate pharmaceutical compositions for animal or human administration. The diluent is selected so as not to affect the biological activity of the combination. Examples of such diluents are distilled water, buffered water, physiological saline, PBS, Ringer's solution, dextrose solution, and Hank's solution. In addition, the pharmaceutical composition or formulation can include other carriers, adjuvants, or non-toxic, nontherapeutic, nonimmunogenic stabilizers, excipients and the like. The compositions can also include additional substances to approximate physiological conditions, such as pH adjusting and buffering agents, toxicity adjusting agents, wetting agents and detergents.

The composition can also include any of a variety of stabilizing agents, such as an antioxidant for example. The polypeptide may be complexed with various well-known compounds that enhance the in vivo stability of the polypeptide, or otherwise enhance its pharmacological properties (e.g., increase the half-life of the polypeptide, reduce its toxicity, enhance solubility or uptake). Examples of such modifications or complexing agents include sulfate, gluconate, citrate and phosphate. The polypeptides of a composition can also be complexed with molecules that enhance their in vivo attributes. Such molecules include, for example, carbohydrates, polyamines, amino acids, other peptides, ions (e.g., sodium, potassium, calcium, magnesium, manganese), and lipids.

Further guidance regarding formulations that are suitable for various types of administration can be found in Remington's Pharmaceutical Sciences, Mace Publishing Company, Philadelphia, Pa., 17th ed. (1985). For a brief review of methods for drug delivery, see, Langer, Science 249:1527-1533 (1990).

The pharmaceutical compositions can be administered for prophylactic and/or therapeutic treatments. Toxicity and therapeutic efficacy of the active ingredient can be determined according to standard pharmaceutical procedures in cell cultures and/or experimental animals, including, for example, determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds that exhibit large therapeutic indices are preferred.

The data obtained from cell culture and/or animal studies can be used in formulating a range of dosages for humans. The dosage of the active ingredient typically lies within a range of circulating concentrations that include the ED₅₀ with low toxicity. The dosage can vary within this range depending upon the dosage form employed and the route of administration utilized.

The pharmaceutical compositions described herein can be administered in a variety of different ways. Examples include administering a composition containing a pharmaceutically acceptable carrier via oral, intranasal, rectal, topical, intraperitoneal, intravenous, intramuscular, subcutaneous, subdermal, transdermal, intrathecal, and intracranial methods.

Formulations suitable for parenteral administration, such as, for example, by intravenous, intramuscular, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives.

The components used to formulate the pharmaceutical compositions are preferably of high purity and are substantially free of potentially harmful contaminants (e.g., at least National Food (NF) grade, generally at least analytical grade, and more typically at least pharmaceutical grade). Moreover, compositions intended for in vivo use are usually sterile. To the extent that a given compound must be synthesized prior to use, the resulting product is typically substantially free of any potentially toxic agents, particularly any endotoxins, which may be present during the synthesis or purification process. Compositions for parental administration are also sterile, substantially isotonic and made under GMP conditions.

### Nucleic Acids

Nucleic acid sequences encoding polypeptides of the invention find use in the recombinant production of the encoded polypeptide, and the like. One of skill in the art can readily utilize well-known codon usage tables and synthetic methods to provide a suitable coding sequence for any of the polypeptides of the invention. Direct chemical synthesis methods include, for example, the phosphotriester method of Narang et al. (1979) Meth. Enzymol. 68: 90-99; the phosphodiester method of Brown et al. (1979) Meth. Enzymol. 68: 109-151; the diethylphosphoramidite method of Beaucage et al. (1981) Tetra. Lett., 22: 1859-1862; and the solid support method of U.S. Pat. No. 4,458,066. Chemical synthesis produces a single stranded oligonucleotide. This can be converted into double stranded DNA by hybridization with a complementary sequence, or by polymerization with a DNA polymerase using the single strand as a template. While chemical synthesis of DNA is often limited to sequences of about 100 bases, longer sequences can be obtained by the ligation of shorter sequences. Alternatively, subsequences may be cloned and the appropriate subsequences cleaved using appropriate restriction enzymes.

The nucleic acids of the subject invention are isolated and obtained in substantial purity, generally as other than an intact chromosome. Usually, the nucleic acids, either as DNA or RNA, will be obtained substantially free of other naturally-occurring nucleic acid sequences, generally being at least about 50%, usually at least about 90% pure and are typically “recombinant,” e.g., flanked by one or more nucleotides with which it is not normally associated on a naturally occurring chromosome. The nucleic acids of the invention can be provided as a linear molecule or within a circular molecule, and can be provided within autonomously replicating molecules (vectors) or within molecules without replication sequences. Expression of the nucleic acids can be regulated by their own or by other regulatory sequences known in the art. The nucleic acids of the invention can be introduced into suitable host cells using a variety of techniques available in the art, such as transferrin polycation-mediated DNA transfer, transfection with naked or encapsulated nucleic acids, liposome-mediated DNA transfer, intracellular transportation of DNA-coated latex beads, protoplast fusion, viral infection, electroporation, gene gun, calcium phosphate-mediated transfection, and the like.

### Methods of Use

The methods of the present invention use any hybrid protein disclosed herein. The hybrid proteins described herein may have one or more of the following characteristics: inhibit activation of the PDGFR signaling pathway and/or VEGFR signaling pathway; diagnose, treat and/or prevent a disease such as an ocular disease, autoimmune disease, inflammatory disease, or cancer. The activities of hybrid proteins may be measured in vitro and/or in vivo.

The present invention provides a method of reducing angiogenesis in a mammal. The method generally involves administering to a mammal a polypeptide of the invention in an amount effective to reduce angiogenesis. An effective amount of an polypeptide of the invention reduces angiogenesis by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, or more, when compared to an untreated (e.g., a placebo-treated) control.

Whether angiogenesis is reduced can be determined using any known method. Methods of determining an effect of an agent on angiogenesis are known in the art and include, but are not limited to, inhibition of neovascularization into implants impregnated with an angiogenic factor; inhibition of blood vessel growth in the cornea or anterior eye chamber; inhibition of endothelial cell proliferation, migration or tube formation in vitro; the chick chorioallantoic membrane assay; the hamster cheek pouch assay; the polyvinyl alcohol sponge disk assay. Such assays are well known in the art and have been described in numerous publications, including, e.g., Auerbach et al. ((1991) Pharmac. Ther. 51:1-11), and references cited therein.

The invention further provides methods for treating a condition or disorder associated with or resulting from pathological angiogenesis. In the context of cancer therapy, a reduction in angiogenesis according to the methods of the invention effects a reduction in tumor size; and a reduction in tumor metastasis. Whether a reduction in tumor size is achieved can be determined, e.g., by measuring the size of the tumor, using standard imaging techniques. Whether metastasis is reduced can be determined using any known method. Methods to assess the effect of an agent on tumor size are well known, and include imaging techniques such as computerized tomography and magnetic resonance imaging.

Any condition or disorder that is associated with or that results from pathological angiogenesis, or that is facilitated by neovascularization (e.g., a tumor that is dependent upon neovascularization), is amenable to treatment with a polypeptide of the invention.

Disclosed herein, in some embodiments, are methods of treating angiogenic-associated conditions in a subject in need thereof. In some embodiments, the angiogenic-associated condition is pterygium. In some embodiments, the angiogenic-associated condition is corneal neovascularization. In some embodiments, the angiogenic-associated condition is pannus. In some embodiments, the angiogenic-associated condition corneal limbal neovascularization from, for instance, contact lens overwear. In some embodiments, the angiogenic-associated condition is pinguecula. In some embodiments, the methods comprise administration of a polypeptide disclosed herein to the subject.

Pterygium (also known as “Surfers Eye”) is a benign vascular growth across the conjunctival and corneal surface of the eye. Pterygium is characterized by a wedge-shaped, highly vascular, fleshy growth that originates on the conjunctiva and that, in some instances, spreads to the corneal limbus and beyond. The pterygium commonly grows from the nasal side of the sclera and is usually present in the palpebral fissure. It is associated with and thought to be caused by ultraviolet-light exposure (e.g., sunlight), low humidity, wind and dust. In some instances, the pterygium is preceded with scleral trauma around the Palpebral comissure. In some instances, the predominance of pterygia on the nasal side is a result of the sun's rays passing laterally through the cornea, where it undergoes refraction and becomes focused on the limbic area. Sunlight passes unobstructed from the lateral side of the eye, focusing on the medial limbus after passing through the cornea. On the contralateral (medial) side, however, the shadow of the nose medially reduces the intensity of sunlight focused on the lateral/temporal limbus.

Pterygium in the conjunctiva is characterized by elastic degeneration of collagen (actinic elastosis) and fibrovascular proliferation. Pterygium generally exhibits neovascularization, remodeling of the extracellular matrix (ECM), and proliferating fibroblasts (FBs). It has an advancing portion called the head of the pterygium, which is connected to the main body of the pterygium by the neck. In some instances, a line of iron deposition is seen adjacent to the head of the pterygium called Stockers line. In some instances, the location of the line gives an indication of the pattern of growth.

Pterygium is composed of several segments: Fuchs' Patches (minute gray blemishes that disperse near the pterygium head), Stockers Line (a brownish line composed of iron deposits), Hood (fibrous nonvascular portion of the pterygium), Head (apex of the pterygium, typically raised and highly vascular), Body (fleshy elevated portion congested with tortuous vessels), Superior Edge (upper edge of the triangular or wing-shaped portion of the pterygium), Inferior Edge (lower edge of the triangular or wing-shaped portion of the pterygium).

In some instances, because pterygium is caused by excessive sun or wind exposure, protective sunglasses with side shields or wide brimmed hats and application of artificial tears to the eyes aids in preventing pterygium formation or prevent further growth.

Additional angiogenic-associated conditions for treatment with the polypeptides disclosed herein include pinguecula, pannus, and corneal neovascularization. Pinguecula is conjunctival degeneration of the eye. Individuals with pinguecula present with yellow-white deposit on the conjunctiva adjacent to the limbus. Histologically, the disorder is characterized by degeneration of the collagen fibers of the conjunctiva stroma with thinning of the overlying epithelium and occasional calcification. Pannus is an abnormal layer of blood vessels into the peripheral cornea. Corneal neovascularization is the excessive ingrowth of blood vessels from the limbal vascular plexus into the cornea often associated with inflammation of or trauma to the cornea.

Treatment with the polypeptides of the present invention can be combined with conventional treatment for pterygium, which include, but are not limited to surgical removal and/or irradiation, conjunctival autografting, amniotic membrane transplantation, or administration of a therapeutic agent. If pterygium recurs after surgery, or is thought to be vision threatening, strontium (⁹⁰Sr) plaque therapy may be used. Conjunctival auto-grafting is an invasive surgical technique for pterygium growth removal. Amniotic membrane transplantation is also used for pterygium growth removal. Other therapeutic agents for the treatment of pterygium include but are not limited to mitomycin C (MMC), 5-fluorouracil (5-FU), loteprednol etabonate (LE), oral doxycycline, dipyridamole, and dobesilate.

Methods of delivering an effective amount of a hybrid protein to a subject are provided herein. The hybrid protein can be delivered to a subject in a composition. The hybrid protein can also be delivered to a subject by a vector comprising a nucleic acid encoding the hybrid protein.

The compositions described herein can be administered to an individual via any route, including, but not limited to, intravenous (e.g., by inhybrid pumps), intraperitoneal, intraocular, intra-arterial, intrapulmonary, oral, inhalation, intravesicular, intramuscular, intra-tracheal, subcutaneous, intraocular, intrathecal, transdermal, transpleural, intraarterial, topical, inhalational (e.g., as mists of sprays), mucosal (such as via nasal mucosa), subcutaneous, transdermal, gastrointestinal, intraarticular, intracisternal, intraventricular, intracranial, intraurethral, intrahepatic, and intratumoral. In some embodiments, the compositions are administered intravascularly, such as intravenously (IV) or intraarterially. In some embodiments, the compositions are administered directly into arteries. In some embodiments, the compositions are administered systemically (for example by intravenous injection). In some embodiments, the compositions are administered locally (for example by intraarterial or intraocular injection).

The dose of a polypeptide of the invention administered to a subject, particularly a human, in the context of the present invention should be sufficient to effect a therapeutic reduction in angiogenesis in the subject over a reasonable time frame. The dose will be determined by, among other considerations, the potency of the particular polypeptide of the invention employed and the condition of the subject, as well as the body weight of the subject to be treated. The size of the dose also will be determined by the existence, nature, and extent of any adverse side-effects that might accompany the administration of a particular compound.

In determining the effective amount of polypeptide in the reduction of angiogenesis, the route of administration, the kinetics of the release system (e.g., pill, gel or other matrix), and the potency of the agonist are considered so as to achieve the desired anti-angiogenic effect with minimal adverse side effects. The polypeptide of the invention will typically be administered to the subject being treated for a time period ranging from a day to a few weeks, consistent with the clinical condition of the treated subject.

As will be readily apparent to the ordinarily skilled artisan, the dosage is adjusted for polypeptide of the invention according to their potency and/or efficacy, for example relative to a currently used VEGF or PDGF antagonist. A dose may be in the range of about 0.001 μg to 100 mg, given 1 to 20 times daily, and can be up to a total daily dose of about 0.01 μg to 100 mg. If applied topically, for the purpose of a systemic effect, the patch or cream would be designed to provide for systemic delivery of a dose in the range of about 0.01 μg to 100 mg. If injected for the purpose of a systemic effect, the matrix in which the polypeptide of the invention is administered is designed to provide for a systemic delivery of a dose in the range of about 0.001 μg to 1 mg. If injected for the purpose of a local effect, the matrix is designed to release locally an amount of polypeptide of the invention in the range of about 0.001 μg to 100 mg.

Regardless of the route of administration, the dose of polypeptide of the invention can be administered over any appropriate time period, e.g., over the course of 1 to 24 hours, over one to several days, etc. Furthermore, multiple doses can be administered over a selected time period. A suitable dose can be administered in suitable subdoses per day, particularly in a prophylactic regimen. The precise treatment level will be dependent upon the response of the subject being treated.

In determining the effective amount of a polypeptide, the route of administration, the kinetics of the release system (e.g., pill, gel or other matrix), and the potency of the agent are considered so as to achieve the desired effect with minimal adverse side effects. The dosage of a polypeptide of the invention is adjusted according to the potency and/or efficacy relative to a VEGF or PDGF antagonist. In some embodiments, a dose is in the range of about 0.001 μg to 100 mg, given 1 to 20 times daily, and be up to a total daily dose of about 0.01 μg to 100 mg. In some embodiments, if applied topically, for the purpose of a systemic effect, the patch or cream is designed to provide for systemic delivery of a dose in the range of about 0.01 μg to 100 mg. In some embodiments, if injected for the purpose of a systemic effect, the matrix in which the polypeptide is administered is designed to provide for a systemic delivery of a dose in the range of about 0.001 μg to 1 mg. If injected for the purpose of a local effect, the matrix is designed to release locally an amount of VEGF variant polypeptide in the range of about 0.001 μg to 100 mg.

In some embodiments, a therapeutically effective amount of a pharmaceutical composition comprising a polypeptide of the invention is expressed as mg polypeptide per kg of subject body mass. In some embodiments, a therapeutically effective amount is 1-1,000 mg/kg, 1-500 mg/kg, 1-250 mg/kg, 1-100 mg/kg, 1-50 mg/kg, 1-25 mg/kg, or 1-10 mg/kg. In some embodiments, an effective amount is 5 mg/kg, 10 mg/kg, 25 mg/kg, 50 mg/kg, 75 mg/kg, 100 mg/kg, 150 mg/kg, 200 mg/kg, 250 mg/kg, 300 mg/kg, 400 mg/kg, 500 mg/kg, 600 mg/kg, 700 mg/kg, 800 mg/kg, 900 mg/kg, 1,000 mg/kg, about 5 mg/kg, about 10 mg/kg, about 25 mg/kg, about 50 mg/kg, about 75 mg/kg, about 100 mg/kg, about 150 mg/kg, about 200 mg/kg, about 250 mg/kg, about 300 mg/kg, about 400 mg/kg, about 500 mg/kg, about 600 mg/kg, about 700 mg/kg, about 800 mg/kg, about 900 mg/kg, or about 1,000 mg/kg.

In some embodiments, a therapeutically effective amount is expressed as mg of the compound per square meter of subject body area. In some embodiments, a pharmaceutical composition comprising a polypeptide is administered subcutaneously in a range of doses, for example 1 to 1500 mg (0.6 to 938 mg/m²), or 2 to 800 mg (1.25 to 500mg/m²), or 5 to 500 mg (3.1 to 312 mg/m²), or 2 to 200 mg (1.25 to 125 mg/m²) or 10 to 1000 mg (6.25 to 625 mg/m²), particular examples of doses including 10 mg (6.25 mg/m²), 20 mg (12.5 mg/m²), 50 mg (31.3 mg/m²), 80 mg (50 mg/m²), 100 mg (62.5 mg/m²), 200 mg (125 mg/m²), 300 mg (187.5 mg/m²), 400 mg (250 mg/m²), 500 mg (312.5 mg/m²), 600 mg (375 mg/m²), 700 mg (437.5 mg/m²), 800 mg (500 mg/m²), 900 mg (562.5mg/m²) and 1000 mg (625 mg/m²).

In some embodiments, while it is possible to use an agent disclosed herein for therapy as is, it is preferable to administer the agent as a pharmaceutical formulation, e.g., in a mixture with a suitable pharmaceutical excipient, diluent, or carrier selected with regard to the intended route of administration and standard pharmaceutical practice. Pharmaceutical formulations include at least one active compound, in association with a pharmaceutically acceptable excipient, diluent, and/or carrier. In some embodiments, the dose and the administration frequency are adjusted based on the judgment of the treating physician, for example taking into account the clinical signs, pathological signs and clinical and subclinical symptoms of a disease of the conditions treated with the present methods, as well as the patient's clinical history. For example, higher doses, increased frequency of administration, or a longer duration of treatment are indicated when a patient is showing symptoms of pterygium or keloid recurrence (e.g., blood vessel growth), or if the patient has a history of previous pterygium or keloid recurrence.

Formulations of polypeptides find use in diagnosis and therapy. In some embodiments, the formulation comprises one, two or more polypeptides or agents. In some embodiments, the therapeutic formulation is administered in combination with other methods of treatment, e.g. chemotherapy, radiation therapy, surgery, and the like.

In some embodiments, formulations are optimized for retention and stabilization at a targeted site. Stabilization techniques include enhancing the size of the polypeptide, by cross-linking, multimerizing, or linking to groups such as polyethylene glycol, polyacrylamide, neutral protein carriers, Fc-fusions etc. in order to achieve an increase in molecular weight. Other strategies for increasing retention include the entrapment of the polypeptide in a biodegradable or bioerodible implant or biogel, or by a non bioerodible polymeric reservoir. The rate of release of the therapeutically active agent is controlled by the rate of transport through the polymeric matrix, and the biodegradation of the implant. The transport of polypeptide through the polymer barrier will also be affected by compound solubility, polymer hydrophilicity, extent of polymer cross-linking, expansion of the polymer upon water absorption so as to make the polymer barrier more permeable to the drug, geometry of the implant, and the like. The implants are of dimensions commensurate with the size and shape of the region selected as the site of implantation. In some embodiments, implants include, e.g., particles, sheets, patches, plaques, fibers, or microcapsules and are any size or shape compatible with the selected insertion site.

In some embodiments, ophthalmic compositions are formulated for pterygium treatment. In some embodiments, the ophthalmic formulation comprises any preparations for conjunctival topical use to be applied to conjunctival mucosa. In some embodiments, the ophthalmic formulation is a liquid preparation (e.g., aqueous or oily solutions or suspensions), or solid preparation (e.g., ointments, powders) for the treatment of an ocular condition, (e.g., pterygium). In some embodiments, the ophthalmic formulation is an ointment. In some embodiments, the ophthalmic formulation is a cream. In some embodiments, other substances are present as excipients in the formulation including anti-oxidant and visco-elastic compounds or vehicles, preservatives, buffer solutions, osmolar and emulsifying substances (or tensioactives).

In some embodiments, the composition comprises one or more excipients such as polyethylene glycol or vaseline and nonionic emulsifying substances (or tensioactives) (such as polysorbate) that could be used for a better tolerability. Ophthalmic formulations for topical use are preferably prepared with a tolerable pH, generally in the range of 6.4-7.8, sterile and devoid of exogenous particles and with a tear-isotonic osmotic pressure around 300 mOsm/L or anywhere between about 200 and about 350 mOsm/L. In some embodiments, the ophthalmic compound is formulated as eye drops, gel, cream or ointment in aqueous or hydro-soluble solvents (e.g., alcohol). Exemplary aqueous solvents include phosphate or citrate phosphate or TRIS buffer, or buffers containing histidine, tricine, lysine, glycine, and/or serine. In some embodiments, solvents are adjusted to the right physiological pH with an acid or basic component. In some embodiments, agents increasing solubility, preservatives, visco-elastic substances (preferably in the range 0.1-10% v/v) (such as hyaluronic acid, polyethylene glycol, mixtures of polyethylene glycol with fatty acids), or celluloses (like hydroxyl-propyl-m ethyl cellulose) are present. Potentially, also anti-oxidant substances, like ascorbic acid in the range 1-15% v/v and chelating agents like EDTA, are contained in the formulation.

In some embodiments, a polypeptide of the invention is administered in a combination therapy with one or more other therapeutic agents, including an inhibitor of angiogenesis; and a cancer chemotherapeutic agent.

In some embodiments, the compositions are administered directly to the eye or the eye tissue. In some embodiments, the compositions are administered topically to the eye, for example, in eye drops. In some embodiments, the compositions are administered by injection to the eye (intraocular injection) or to the tissues associated with the eye. The compositions can be administered, for example, by intraocular injection, intralesional injection, periocular injection, subretinal injection, intravitreal injection, trans-septal injection, subscleral injection, intrachoroidal injection, intracameral injection, subconjunctival injection, sub-Tenon's injection, retrobulbar injection, peribulbar injection, intradermal injection, subcutaneous injection, or posterior juxtascleral delivery. These methods are known in the art. For example, for a description of exemplary periocular routes for retinal drug delivery, see Raghava et al., Expert Opin. Drug Deliv., 2004, 1(1):99-114. The compositions may be administered, for example, to the vitreous, aqueous humor, sclera, conjunctiva, the area between the sclera and conjunctiva, the retina choroids tissues, macula, or other area in or proximate to the eye of an individual. The compositions can also be administered to the individual as an implant. Preferred implants are biocompatible and/or biodegradable sustained release formulations which gradually release the compounds over a period of time. Ocular implants for drug delivery are well-known in the art. See, e.g., U.S. Pat. Nos. 5,501,856, 5,476,511, and 6,331,313. The compositions can also be administered to the individual using iontophoresis, including, but are not limited to, the ionophoretic methods described in U.S. Pat. No. 4,454,151 and U.S. Pat. App. Pub. No. 2003/0181531 and 2004/0058313.

The optimal effective amount of the compositions can be determined empirically and will depend on the type and severity of the disease, route of administration, disease progression and health, mass and body area of the individual. Such determinations are within the skill of one in the art.

Compositions comprising a hybrid protein may be administered in a single daily dose, or the total daily dose may be administered in divided dosages of two, three, or four times daily. Compositions comprising a hybrid protein can also be administered six times a week, five times a week, four times a week, three times a week, twice a week, once a week, once every two weeks, once every three weeks, once a month, once every two months, once every three months, once every six months, once every nine months, or once every year.

The compositions may also be administered in a sustained release formulation, such as in an implant which gradually releases the composition for use over a period of time, and which allows for the composition to be administered less frequently, such as once a month, once every 2-6 months, once every year, or even a single administration. The sustained release devices (such as pellets, nanoparticles, microparticles, nanospheres, microspheres, and the like) may be administered by injection or surgical implanted in various locations in the eye or tissue associated with the eye, such as intraocular, intravitreal, subretinal, periocular, subconjunctival, or sub-Tenons.

Compositions of the invention can be used either alone or in combination with one or more additional therapeutic agents. For example, the compositions of the invention can be administered alone or in combination with other therapeutic agents known to have a beneficial effect on age-related macular degeneration (AMD), retinal attachment or damaged retinal tissue. Exemplary therapeutic agents include complement inhibitors, anti-angiogenics, anti-VEGF agents (including, but not limited to Macugen (pegaptanib sodium), Eylea® (VEGF Trap-Eye), and anti-VEGF antibody, such as Lucentis® or Avastin®), and anti-PDGF agents (such as Fostiva®). The compositions of the invention can be administered in combination with nutritional supplements shown to be beneficial in lowering the risk of macular degeneration progressing to advanced stages, e.g., vitamin C, vitamin E, beta carotene, zinc oxide, and copper. Other useful cofactors include symptom-alleviating cofactors, including antiseptics, antibiotics, antiviral and antifungal agents, and analgesics and anesthetics. In some embodiments, a combination is provided as a concomittant administration. In some embodiments, a combination is provided as a separate administration where the additional therapeutic agent is administered prior to, simultaneously, and/or following administration of the hybrid protein. The interval between sequential administration can be in terms of at least (or, alternatively, less than) minutes, hours, or days.

The compositions described herein can also be used in conjunction with other AMD therapies, such as photodynamic therapy. Photodynamic therapy entails the intravenous administration of Visudyne (verteporfin), after which light of a specific wavelength is applied to the abnormal blood vessels. The light activates the Visudyne and obliterates the vessels. Alternatively, the compositions described herein can be used in conjunction with laser therapy, which entails using a high-energy laser beam to destroy abnormal blood vessels under the macula.

Suitable chemotherapeutic agents include, but are not limited to, the alkylating agents, e.g. Cisplatin, Cyclophosphamide, Altretamine; the DNA strand-breakage agents, such as Bleomycin; DNA topoisomerase II inhibitors, including intercalators, such as Amsacrine, Dactinomycin, Daunorubicin, Doxorubicin, Idarubicin, and Mitoxantrone; the nonintercalating topoisomerase II inhibitors such as, Etoposide and Teniposide; the DNA minor groove binder Plicamycin; alkylating agents, including nitrogen mustards such as Chlorambucil, Cyclophosphamide, Isofamide, Mechlorethamine, Melphalan, Uracil mustard; aziridines such as Thiotepa; methanesulfonate esters such as Busulfan; nitroso ureas, such as Carmustine, Lomustine, Streptozocin; platinum complexes, such as Cisplatin, Carboplatin; bioreductive alkylator, such as Mitomycin, and Procarbazine, Dacarbazine and Altretamine; antimetabolites, including folate antagonists such as Methotrexate and trimetrexate; pyrimidine antagonists, such as Fluorouracil, Fluorodeoxyuridine, CB3717, Azacytidine, Cytarabine; Floxuridine purine antagonists including Mercaptopurine, 6-Thioguanine, Fludarabine, Pentostatin; sugar modified analogs include Cyctrabine, Fludarabine; ribonucleotide reductase inhibitors including hydroxyurea; Tubulin interactive agents including Vincristine Vinblastine, and Paclitaxel; adrenal corticosteroids such as Prednisone, Dexamethasone, Methylprednisolone, and Prodnisolone; hormonal blocking agents including estrogens, conjugated estrogens and Ethinyl Estradiol and Diethylstilbesterol, Chlorotrianisene and Idenestrol; progestins such as Hydroxyprogesterone caproate, Medroxyprogesterone, and Megestrol; androgens such as testosterone, testosterone propionate; fluoxymesterone, methyltestosterone estrogens, conjugated estrogens and Ethinyl Estradiol and Diethylstilbesterol, Chlorotrianisene and Idenestrol; and the like.

The polypeptide of the invention may be administered with other anti-angiogenic agents. Anti-angiogenic agents include, but are not limited to, angiostatic steroids such as heparin derivatives and glucocorticosteroids; thrombospondin; cytokines such as IL-12; fumagillin and synthetic derivatives thereof, such as AGM 12470; interferon-a; endostatin; soluble growth factor receptors; neutralizing monoclonal antibodies directed against growth factors such as vascular endothelial growth factor; and the like.

Disclosed herein, in some embodiments, are methods of treating an ocular disorder, for example pterygium, in a subject in need thereof. In some embodiments, the methods comprise administration of a polypeptide of the present invention and an additional therapeutic agent to the subject. In some embodiments, the additional therapeutic agent is an inhibitor of a vascular endothelial growth factor (VEGF), a platelet-derived growth factor (PDGF), a fibroblast growth factor (FGF), or an angiotensin (ANG), and associated receptors. In some embodiments, the additional therapeutic agent is an inhibitor of an integrin, or an inhibitor of a matrix metalloproteinase (MMP), or prostate specific membrane antigen (PSMA). In some embodiments, the additional therapeutic is selected from the group consisting of an antibody, polypeptide, nucleotide, a small molecule, and combinations thereof. In some embodiments, the additional therapeutic agent is selected from the group consisting of: mitomycin C (MMC), 5-fluorouracil (5-FU), loteprednol etabonate (LE), oral doxycycline, dipyridamole, and dobesilate. In some embodiments, the additional therapeutic agent is an anti-inflammatory steroid. In some embodiments, the additional therapeutic agent is non-steroidal anti-inflammatory agent. In some embodiments, the additional therapeutic agent is an antibody or small molecule inhibitor of VEGF signaling. In some embodiments, the additional therapeutic agent binds, traps, scavenges or otherwise deters the effect of VEGF that has already been produced.

In some embodiments, the polypeptide of the present invention and the additional therapeutic agent are administered in a unified dosage form or in separate dosage forms. In some embodiments, the methods comprise administration of a polypeptide disclosed herein in combination with a therapeutic procedure. Procedures that provide additional or synergistic benefit include, but are not limited to irradiation (e.g. ⁹⁰Sr therapy), conjunctival autografting or amniotic membrane transplantation, or surgery.

By way of example only, the therapeutic effectiveness of one of the therapeutic agents described herein is enhanced by administration of an adjuvant (i.e., by itself the adjuvant has minimal therapeutic benefit, but in combination with another therapeutic agent, the overall therapeutic benefit to the patient is enhanced). Or, by way of example only, the benefit experienced by an individual is increased by administering one of the therapeutic agents described herein with another therapeutic agent (which also includes a therapeutic regimen) that also has therapeutic benefit. In any case, regardless of the disease or disorder being treated, the overall benefit experienced by the patient is simply additive of the two therapeutic agents or in other embodiments, the patient experiences a synergistic benefit.

The particular choice of agents used will depend upon the diagnosis of the attending physicians and their judgment of the condition of the patient and the appropriate treatment protocol. The agents are optionally administered concurrently (e.g., simultaneously, essentially simultaneously or within the same treatment protocol) or sequentially, depending upon the nature of the disorder, the condition of the patient, and the actual choice of agents used. The determination of the order of administration, and the number of repetitions of administration of each therapeutic agent during a treatment protocol, is based on an evaluation of the disease being treated and the condition of the patient.

In some embodiments, therapeutically-effective dosages vary when the drugs are used in treatment combinations. Methods for experimentally determining therapeutically-effective dosages of drugs and other agents for use in combination treatment regimens are described in the literature. For example, the use of metronomic dosing, i.e., providing more frequent, lower doses in order to minimize toxic side effects, has been described extensively in the literature. Combination treatment further includes periodic treatments that start and stop at various times to assist with the clinical management of the patient.

In another aspect, a pharmaceutical composition comprising a polypeptide of the present invention is incorporated into an ophthalmic device that comprises a biodegradable material, and the device is implanted into a subject to provide a long-term (e.g., longer than about 1 week, or longer than about 1, 2, 3, 4, 5, or 6 months) treatment of the ocular condition, such as pterygium. Such a device is implanted by a skilled physician in the subject's ocular or periocular tissue.

The methods of treating conditions with a pharmaceutical composition comprising a polypeptide described herein offer advantages both over surgical methods of treatment and over existing biologic agents. No non-surgical intervention exists for early or advanced pterygium. Furthermore, even if entirely successful in removal of the vascular and fibrous tissue components, surgery cannot prevent the recurrence of pterygium. Repeat invasive surgeries for excision of pterygium carry significant risks. Hence, a pharmaceutical composition comprising a polypeptide that controls the growth of existing pterygium and/or prevent the recurrence of pterygium post-surgical excision are advantageous. In some embodiments, a pharmaceutical composition comprising a polypeptide of the present invention is administered during and/or immediately after surgery, such as by intralesional injection, subconjunctival injection, or other direct application to or near the pterygium site. In some embodiments, a course of treatment combines elements of the above, such as administration during and/or after surgery by injection or other technique, plus at-home (out-of-office) administered eye drops or other means of topical administration in the days, weeks, and/or months after surgery. In some embodiments, a pharmaceutical composition comprising a polypeptide of the present invention is used to treat a condition instead of surgery, to halt progression or induce regression of the condition. If the pharmaceutical composition comprising a polypeptide of the present invention is shown to be particularly effective, then patients and physicians, who might have otherwise opted for pterygium surgery, might opt for treatment with a pharmaceutical composition alone instead of surgery, to avoid the cost, time, pain, and risk of surgery. Other patient classes that would benefit from a pharmaceutical composition without surgery include those that don't qualify for surgery, those that can't afford surgery, and those who qualify for but choose to not undergo surgery. Second, a pharmaceutical composition could be used during and/or after surgery, to prevent recurrence, particularly because of unacceptably high recurrence rates in past and present techniques, or the need for very complex forms of surgery that include ocular tissue transplantation or transfer.

In some embodiments, a method of treatment involves professional intervention combined with administration of a pharmaceutical composition comprising a polypeptide of the present invention. For example, in some embodiments, a method of treatment first involves debridement of the surface layer of a pterygium such as the epithelium or superficial fibroblastic layer, followed by administration of a pharmaceutical composition comprising a polypeptide of the present invention. The administration can be topical or intralesional. In some embodiments, debridement is a simpler, less expensive, shorter, and lower-risk intervention that enables or enhances the effect of a pharmaceutical composition, such as by exposing endothelial cells, fibroblasts, or other cells to the anti-angiogenic, anti-growth, and/or anti-migratory effects of the polypeptide or otherwise enhancing their penetration into the lesion.

Existing biologics target only a subset of ligand-receptor interactions that mediate angiogenesis which inherently limits their efficacy. In some embodiments, the polypeptides described herein target multiple receptors and exhibit superior efficacy compared to agents that target fewer, or a single target. Furthermore, the polypeptide compositions utilize a soluble growth factor scaffold, and are significantly smaller in size (25 kDa) when compared to existing biologics (50-150 kDa) which are either antibodies, antibody fragments or receptor extra-cellular domains fused to antibody Fc domains. Accordingly, whereas the large size of the existing biologics necessitates delivery via injection (subconjunctival), in some embodiments, a pharmaceutical composition comprising a polypeptide described herein is administered topically. This represents a significant reduction in patient compliance burden and the cost of therapy.

Ideally, a treatment for pterygium, whether post-surgery, to reduce rates of recurrence, or instead of surgery, to halt progression or induce regression, would be easily and safely administered, such as topical eye drops or other similar formulations such as viscous gels, or ointments. A preferred method of treatment is a topical eye drop, self-administered as infrequent as once per course of treatment or once per month. Less preferred, but still very satisfactory, is more frequent self-administered topical formulations, since that still avoids the time, cost, pain, and risk of injections. For example, eye drops, gels or ointments applied out-of-office once per week, twice per week, once per day, or twice per day, or three times per day or four times per day.

The present invention provides methods of treating a disease (such as an ocular disease, an inflammatory disease, an autoimmune disease, or cancer) by administering an effective amount of any hybrid protein described herein to an individual. In some embodiments, a method of treating a disease comprises administering an effective amount of a composition comprising the hybrid protein to an individual. Methods of treating or preventing one or more aspects or symptoms of a disease (such as an ocular disease, an inflammatory disease, an autoimmune disease, or cancer) by administering an effective amount of any hybrid protein described herein to an individual are also provided. In some embodiments, a method of treating or preventing one or more aspects or symptoms of a disease comprises administering an effective amount of a composition comprising the hybrid protein to an individual. In some embodiments, a method of treating or preventing one or more aspects or symptoms of a disease comprises administering an effective amount of a vector comprising a nucleic acid encoding the hybrid protein to an individual.

The methods described herein can be used for the treatment of a variety of diseases, including, but not limited to, inflammatory disease, ocular disease, autoimmune disease, or cancer. In some embodiments, the disease to be treated includes, but is not limited to, rheumatoid arthritis, inflammatory arthritis, osteoarthritis, cancer, age-related macular degeneration (AMD) (such as wet AMD or dry AMD), ocular disease characterized by neovascularization (such as choroidal neovascularization), uveitis (such as anterior uveitis or posterior uveitis), retinitis pigmentosa, and diabetic retinopathy.

In certain embodiments, the methods and compositions of the invention can be used to treat an autoimmune disease. In some embodiments, the autoimmune disease is rheumatoid arthritis, multiple sclerosis, or systemic lupus erythematosus. Rheumatoid arthritis (RA) is a chronic autoimmune disease that leads to inflammation of the joints. While RA principally affects synovial joints, it can affect surrounding tissues and organs. The pathology of RA involves an inflammatory process that can lead to the destruction of cartilage and the ankylosis (hybrid) of joints. Other pathological manifestations of RA include vasculitis (inflammation of the blood vessels), which can affect nearly any organ system and can cause additional complications, including polyneuropathy, cutaneous ulceration, and visceral infarction. Pleuropulmonary manifestations include pleuritis, interstitial fibrosis, Caplan's syndrome, pleuropulmonary nodules, pneumonitis, rheumatoid lung disease and arteritis. Other manifestations include the development of inflammatory rheumatoid nodules on a variety of periarticular structures such as extensor surfaces, as well as on pleura and meninges. Weakness and atrophy of skeletal muscle are common.

In certain embodiments, the methods and compositions of the invention can be used to treat an inflammatory disease. In some embodiments, the inflammatory disease is inflammatory arthritis, osteoarthritis, psoriasis, chronic inflammation, irritable bowel disease, lung inflammation or asthma. Inflammatory arthritis refers to inflammation of the joints that can result from an autoimmune disease such as, e.g., ankylosing spondylitis, juvenile idiopathic arthritis, mixed connective tissue disease, psoriatic arthritis, reactive arthritis, scleroderma, Sjogren's Syndrome, Still's Disease, and systemic lupus erythematosus. Inflammatory arthritis can also be caused by certain types of bacteria (such as with reactive arthritis) or by deposits of crystalline structures in the joints (such as with gout and pseudogout). The characteristic symptoms of inflammatory arthritis are pain and swelling of one or more joints, which may be warmer than the other joints. Stiffness of the joints following prolonged inactivity (such as in the morning or after sitting for a length of time) is a very common symptom. Patients with inflammatory arthritis usually have multiple joint complaints. Osteoarthritis, also known as degenerative arthritis or degenerative joint disease, is a group of mechanical abnormalities involving degradation of joints, including articular cartilage and subchondral bone. Symptoms may include joint pain, tenderness, stiffness, locking. A variety of causes, e.g., hereditary, developmental, metabolic, obesity-related, and mechanical, may initiate processes leading to loss of cartilage. As breakdown products from the cartilage are released into the synovial space, the cells lining the joint attempt to remove them. New bone outgrowths, or “spurs” can form. Often, when bone becomes less well protected by cartilage, bone may be exposed and damaged. These bone changes, in combination with inflammation of the joint, cause pain. As a result of decreased movement secondary to pain, regional muscles may atrophy, and ligaments may become more lax.

Persistent and unregulated angiogenesis occurs in a multiplicity of disease states such as cancer. In cancer, cells divide and grow uncontrollably, forming malignant tumors, which vascularize and invade nearby parts of the body. The cancer may also spread (metastasize) to more distant parts of the body through the lymphatic system or bloodstream. The causes of cancer can be environmental (due to exposure to chemicals, radiation or due to lifestyle), hereditary, or infectious. In some embodiments, the methods and compositions of the invention can be used to treat cancer. In some embodiments, the cancer is prostate cancer, breast cancer, lung cancer, esophageal cancer, colon cancer, rectal cancer, liver cancer, urinary tract cancer (e.g., bladder cancer), kidney cancer, lung cancer (e.g., non-small cell lung cancer), ovarian cancer, cervical cancer, endometrial cancer, pancreatic cancer, stomach cancer, thyroid cancer, skin cancer (e.g., melanoma), hematopoietic cancers of lymphoid or myeloid lineage, head and neck cancer, nasopharyngeal carcinoma (NPC), glioblastoma, teratocarcinoma, neuroblastoma, adenocarcinoma, cancers of mesenchymal origin such as a fibrosarcoma or rhabdomyosarcoma, soft tissue sarcoma and carcinoma, choriocarcinioma, hepatoblastoma, Karposi's sarcoma or Wilm's tumor.

Other diseases that are associated with angiogenesis can be treated with the methods and compositions disclosed herein. These diseases include atherosclerosis, retrolentral fibroplasia, thyroid hyperplasias (including grave's disease), nephrotic syndrome, preclampasia, ascites, pericardial effusion (such as associated with pericarditis) and pleural effusion.

In some embodiments, the methods and compositions of the invention can be used to treat an ocular disease. In some embodiments, the ocular disease is AMD such as wet AMD or dry AMD, uveitis, retinitis pigmentosa, neovascular glaucoma, diabetic retinopathy, and other eye diseases that involve a local inflammatory process. In some embodiments, the ocular disease is characterized by neovascularization, such as choroidal neovascularization. In some embodiments, the ocular disease is a result of corneal transplantation. In some embodiments, the invention provides methods of treating or preventing one or more aspects or symptoms of an ocular disease including, but not limited to, formation of ocular drusen, inflammation in the eye or eye tissue and loss of vision. In certain embodiments, the compositions and methods described herein can be used to detect and/or treat uveitis, i.e., inflammation of the uvea, the middle layer of the eye beneath the sclera. Uveitis is estimated to be responsible for approximately 10%-20% of the blindness in the United States. The uvea is traditionally divided into 3 areas, from front to back, the iris, ciliary body, and choroid. The prime functions of the uvea are nutrition and gas exchange, light absorption, and secretion of the aqueous humour by the cilliary processes. Uveitis is typically associated with exposure to toxins, infection, and/or autoimmune disorders. However, in many cases, the cause is unknown. Uveitis can affect one or both eyes. Symptoms may develop rapidly and can include blurred vision, floating dark spots in the field of vision, eye pain, eye redness, and sensitivity to light. The most common form of uveitis is anterior uveitis, or iritis, which involves inflammation of the iris. Pars plantis refers to inflammation of the uvea in the middle of the eye, i.e., between the iris and the choroid. Posterior uveitis affects the back of the eye, i.e., the choroid. Inflammation associated with posterior uveitis can also affect the retina (retinitis) or the blood vessels at the back of the eye (vasculitis).

In certain embodiments, the methods and compositions of the invention can be used to treat retinitis pigmentosa (RP). RP is a heritable eye disease that is caused by abnormalities of the photoreceptors (rods and cones) or the retinal pigment epithelium of the retina. The disease can lead to progressive sight loss and often blindness. The symptoms of RP include decreased vision at night or in low light, loss of side (peripheral) vision, and, in advanced cases, loss of central vision. The diagnosis of RP relies upon the documentation of progressive loss in photoreceptor cell function via visual field testing and electroretinography. At least 35 genetic loci are known to cause “non-syndromic retinitis pigmentosa” (i.e., RP that is not the result of another disease or part of a wider syndrome).

In certain embodiments, the methods and compositions of the invention can be used to treat diabetic retinopathy. Diabetic retinopathy refers to damage to the retina caused by the complications of diabetes. Specifically, vascular walls are compromised by hyperglycemia, changing the formation of the blood-retinal barrier and making the retinal blood vessels more permeable. The damaged blood vessels leak fluid and lipids into the macula, causing the macular to swell (i.e., macular edema), which blurs vision. As the disease progresses, it enters a proliferative stage, in which blood vessels grow along the retina and in the vitreous humour that fills the eye. These blood vessels can bleed, cloud vision, and e.g., destroy the retina, cause retinal detachment, or cause neovascular glaucoma.

In certain embodiments, the methods and compositions of the invention can be used to treat age-related macular degeneration (AMD). AMD is characterized by progressive loss of central vision which occurs as a result of damage to the photoreceptor cells in an area of the retina called the macula. AMD has been broadly classified into two clinical states: a wet form and a dry form, with the dry form making up to 80-90% of total cases. Dry AMD is characterized by the formation of macular drusen, tiny yellow or white accumulations of extracellular material that builds up between Bruch's membrane and the retinal pigment epithelium of the eye. Wet AMD, which accounts for approximately 90% of serious vision loss, is associated with neovascularization, wherein blood vessels grow up from the choroid beneath the retina, and with the leakage of these new vessels. The accumulation of blood and fluid can cause retinal detachment followed by rapid photoreceptor degeneration and loss of vision in either form of AMD. It is generally accepted that the wet form of AMD is preceded by and arises from the dry form.

In some embodiments, a pharmaceutical composition comprising a polypeptide of the present invention is administered to a patient on a regular basis, e.g., three times a day, two times a day, once a day, every other day or every 3 days. In other embodiments, a pharmaceutical composition comprising a polypeptide of the present invention is administered to the patient on an intermittent basis, e.g., twice a day followed by once a day followed by three times a day; or the first two days of every week; or the first, second and third day of a week. In some embodiments, intermittent dosing is as effective as regular dosing. In the case wherein the patient's condition does not improve, upon the doctor's discretion the administration of a pharmaceutical composition comprising a polypeptide of the present invention is administered chronically, that is, for an extended period of time, including throughout the duration of the patient's life in order to ameliorate or otherwise control or limit the symptoms of the patient's disorder.

In the case wherein the patient's status does improve, upon the doctor's discretion the administration of a pharmaceutical composition comprising a polypeptide of the present invention is given continuously; alternatively, the dose of drug being administered is temporarily reduced or temporarily suspended for a some length of time (i.e., a “drug holiday”). In some embodiments, the length of the drug holiday varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday is from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

### Imaging

Disclosed herein, in certain embodiments, are methods for diagnosing an angiogenic disorder in a subject in need thereof comprising: (a) contacting a biological sample from the subject with a labelled hybrid polypeptide of the invention that binds to a biomarker; (b) determining the amount of the biomarker in the biological sample by measuring the amount of the labelled VEGF variant polypeptide bound to the biomarker; (c) comparing the determined amount of the biomarker in the biological sample to an amount of the biomarker in a control; and (d) diagnosing the subject as having an angiogenic disorder based on the comparison.

In some embodiments, the labelling agent comprises a label, a dye, a photocrosslinker, a cytotoxic compound, a drug, an affinity label, a photoaffinity label, a reactive compound, an antibody or antibody fragment, a biomaterial, a nanoparticle, a spin label, a fluorophore, a metal-containing moiety, a radioactive moiety, a novel functional group, a group that covalently or noncovalently interacts with other molecules, a photocaged moiety, an actinic radiation excitable moiety, a ligand, a photoisomerizable moiety, biotin, a biotin analog, a moiety incorporating a heavy atom, a chemically cleavable group, a photocleavable group, a redox-active agent, an isotopically labeled moiety, a biophysical probe, a phosphorescent group, a chemiluminescent group, an electron dense group, a magnetic group, an intercalating group, a chromophore, an energy transfer agent, a biologically active agent, a detectable label, or a combination thereof. In some embodiments, the fluorophore is selected from the group consisting of BODIPY 493/503, BODIPY FL, BODIPY R6G, BODIPY 530/550, BODIPY TMR, BODIPY 558/568, BODIPY 564/570, BODIPY 576/589, BODIPY 581/591, BODIPY TR, Fluorescein, 5(6)-Carboxyfluorescein, 2,7 -Dichlorofluorescein, N,N-Bis(2,4,6-trimethylphenyI)-3,4: 9,10-perylenebis(dicarboximide, HPTS, Ethyl Eosin, DY-490XL MegaStokes, DY-485XL MegaStokes, Adirondack Green 520, ATTO 465, ATTO 488, ATTO 495, YOYO-1, 5-FAM, BCECF, BCECF, dichlorofluorescein, rhodamine 110, rhodamine 123, Rhodamine Green, YO-PRO-1, SYTOX Green, Sodium Green, SYBR Green I, Alexa Fluor 500, FITC, Fluo-3, Fluo-4, fluoro-emerald, YoYo-1 ssDNA, YoYo-1 dsDNA, YoYo-1, SYTO RNASelect, Diverse Green-FP, Dragon Green, EvaGreen, Surf Green EX, Spectrum Green, Oregon Green 488, NeuroTrace 500525, NBD-X, MitoTracker Green FM, LysoTracker Green DND-26, CBQCA, PA-GFP (post-activation), WEGFP (post-activation), FIASH-CCXXCC, Azami Green monomeric, Azami Green, EGFP (Campbell Tsien 2003), EGFP (Patterson 2001), Fluorescein, Kaede Green, 7-Benzylamino-4-Nitrobenz-2-Oxa-1,3-Diazole, Bex1, Doxorubicin, Lumio Green, IRDye 800, IRDye 750, IRDye 700, DyLight 680, DyLight 755, DyLight 800 and SuperGlo GFP. In some embodiments, the labelling agent is selected from the group consisting of: a positron-emitting isotope (such as ¹⁸F), a gamma-ray isotope (such as ^{99m}Tc), a paramagnetic molecule or nanoparticle (such as a coated magnetite nanoparticle), a gadolinium chelate (such as diethylene triamine pentaacetic acid (DTPA), 1,4,7,10-tetraazacyclododecane-1,4,7,10-tetraacetic acid (DOTA), and 1,4,7-triazacyclononane-N,N′,N″-triacetic acid (NOTA)), an iron oxide particle, a super paramagnetic iron oxide particle, an ultra small paramagnetic particle, a manganese chelate, a gallium containing agent, a technetium chelate (such as HYNIC, DTPA, and DOTA), a copper chelate, a radioactive fluorine, a radioactive iodine, a indiuim chelate, or a radioactive moiety (such as ²¹¹At, ¹³¹I, ¹²⁵I, ⁹⁰Y, ¹⁸⁶Re, ¹⁸⁸Re, ¹⁵³Sm, ²¹²Bi, ³²P, ⁶⁴Cu radioactive isotopes of Lu). In some embodiments, the connecting moiety connects the labelling agent to the VEGF variant polypeptide. In some embodiments, the connecting moiety is selected from the group consisting of a bond, a peptide, a polymer, a water soluble polymer, optionally substituted alkyl, optionally substituted heteroalkyl, optionally substituted heterocycloalkyl, optionally substituted cycloalkyl, optionally substituted heterocycloalkylalkyl, optionally substituted heterocycloalkylalkenyl, optionally substituted aryl, optionally substituted heteroaryl, and optionally substituted heterocycloalkylalkenylalkyl. In some embodiments, the connecting moiety is 4′-phosphopantetheine.

In some embodiments, the fluorophore is selected from the group consisting of: BODIPY 493/503, BODIPY FL, BODIPY R6G, BODIPY 530/550, BODIPY TMR, BODIPY 558/568, BODIPY 564/570, BODIPY 576/589, BODIPY 581/591, and BODIPY TR. In some embodiments, the fluorophore is BODIPY FL. In some embodiments, the fluorophore is not BODIPY 530. In some embodiments, the fluorophore has an excitation maxima of between about 500 and about 600 nm. In some embodiments, the fluorophore has an excitation maxima of between about 500 and about 550 nm. In some embodiments, the fluorophore has an excitation maxima of between about 550 and about 600 nm. In some embodiments, the fluorophore has an excitation maxima of between about 525 and about 575 nm. In some embodiments, the fluorophore has an emission maxima of between about 510 and about 670 nm. In some embodiments, the fluorophore has an emission maxima of between about 510 and about 600 nm. In some embodiments, the fluorophore has an emission maxima of between about 600 and about 670 nm. In some embodiments, the fluorophore has an emission maxima of between about 575 and about 625 nm.

In some embodiments, the fluorophore is fluorescein or indocyanine green.

In some embodiments, the fluorophore is ATTO 488, DY-547 or DY-747.

In some embodiments, the labelling agent is a positron-emitting isotope (e.g.,¹⁸F) for positron emission tomography (PET), gamma-ray isotope (e.g., ^{99m}Tc) for single photon emission computed tomography (SPECT), or a paramagnetic molecule or nanoparticle (e.g., Gd³⁺ chelate or coated magnetite nanoparticle) for magnetic resonance imaging (MRI).

In some embodiments, the labelling agent is: a gadolinium chelate, an iron oxide particle, a super paramagnetic iron oxide particle, an ultra small paramagnetic particle, a manganese chelate or gallium containing agent. Examples of gadolinium chelates include, but are not limited to diethylene triamine pentaacetic acid (DTPA), 1,4,7,10-tetraazacyclododecane-1,4,7,10-tetraacetic acid (DOTA), and 1,4,7-triazacyclononane-N,N′,N″-triacetic acid (NOTA).

In some embodiments, the labelling agent is a near-infrared fluorophore for near-infra red (near-IR) imaging, a luciferase (firefly, bacterial, or coelenterate) or other luminescent molecule for bioluminescence imaging, or a perfluorocarbon-filled vesicle for ultrasound.

In some embodiments, the labelling agent is a nuclear probe. In some embodiments, the imaging agent is a SPECT or PET radionuclide probe. In some embodiments, the radionuclide probe is selected from: a technetium chelate, a copper chelate, a radioactive fluorine, a radioactive iodine, a indiuim chelate. Examples of Tc chelates include, but are not limited to HYNIC, DTPA, and DOTA.

In some embodiments, the labelling agent is a radioactive moiety, for example a radioactive isotope such as ²¹¹At, ¹³¹I, ¹²⁵I, ⁹⁰Y, ¹⁸⁶Re, ¹⁸⁸Re, ¹⁵³Sm, ²¹²Bi, ³²P, ⁶⁴Cu radioactive isotopes of Lu, and others.

In some embodiments, the polypeptide of the invention further comprises a Sfp tag that is at least 90%, at least 95%, at least 99%, or 100% identical to a peptide sequence of DSLEFIASKLA.

In some embodiments, a labelled hybrid polypeptide of the invention comprises the hybrid polypeptide, a connecting moiety, and a labelling agent. In some embodiments, the connecting moiety connects the labelling agent to the polypeptide. In some embodiments, the connecting moiety is selected from a bond, a peptide, a polymer, a water soluble polymer, optionally substituted alkyl, optionally substituted heteroalkyl, optionally substituted heterocycloalkyl, optionally substituted cycloalkyl, optionally substituted heterocycloalkylalkyl, optionally substituted heterocycloalkylalkenyl, optionally substituted aryl, optionally substituted heteroaryl, and optionally substituted heterocycloalkylalkenylalkyl. In some embodiments, the connecting moiety is an optionally substituted heterocycle. In some embodiments, the heterocycle is selected from aziridine, oxirane, episulfide, azetidine, oxetane, pyrroline, tetrahydrofuran, tetrahydrothiophene, pyrrolidine, pyrazole, pyrrole, imidazole, triazole, tetrazole, oxazole, isoxazole, oxirene, thiazole, isothiazole, dithiolane, furan, thiophene, piperidine, tetrahydropyran, thiane, pyridine, pyran, thiapyrane, pyridazine, pyrimidine, pyrazine, piperazine, oxazine, thiazine, dithiane, and dioxane. In some embodiments, the heterocycle is piperazine. In further embodiments, the connecting moiety is optionally substituted with a halogen, CN, OH, NO₂, alkyl, S(0), and S(0)₂. In other embodiments, the water soluble polymer is a PEG group.

In some embodiments, the angiogenic disorder is ocular neovascularization, choroidal neovascularization, iris neovascularization, corneal neovascularization, retinal neovascularization, pterygium, pannus, pinguecula, diabetic retinopathy, diabetic macular edema, retinal detachment, posterior uveitis, macular degeneration, a keloid, glaucoma, cataract, partial blindness, complete blindness, myopia, myopic degeneration, deterioration of central vision, metamophospsia, color disturbances, hemorrhaging of blood vessels, or retinal vein occlusion.

In some embodiments, the angiogenic disorder is a cancer. In some embodiments, the cancer is prostate cancer, breast cancer, lung cancer, esophageal cancer, colon cancer, rectal cancer, liver cancer, urinary tract cancer (e.g., bladder cancer), kidney cancer, lung cancer (e.g., non-small cell lung cancer), ovarian cancer, cervical cancer, endometrial cancer, pancreatic cancer, stomach cancer, thyroid cancer, skin cancer (e.g., melanoma), hematopoietic cancers of lymphoid or myeloid lineage, head and neck cancer, nasopharyngeal carcinoma (NPC), glioblastoma, teratocarcinoma, neuroblastoma, adenocarcinoma, cancers of mesenchymal origin such as a fibrosarcoma or rhabdomyosarcoma, soft tissue sarcoma and carcinoma, choriocarcinioma, hepatoblastoma, Karposi's sarcoma or Wilm's tumor.

In some embodiments, the angiogenic disorder is an inflammtory disorder. In some embodiments, the inflammatory disorder is inflammatory arthritis, osteoarthritis, psoriasis, chronic inflammation, irritable bowel disease, lung inflammation or asthma. In some embodiments, the angiogenic disorder is an autoimmune disorder. In some embodiments, the autoimmune disorder is rheumatoid arthritis, multiple sclerosis, or systemic lupus erythematosus.

In some embodiments, the biomarker is a biomarker of an angiogenic disorder. In some embodiments, the growth factor receptor is a vascular endothelial growth factor receptor (VEGFR). In some embodiments, the VEGFR is VEGFR1 or VEGFR2. In some embodiments the growth factor receptor is PDGFR-α or PDGFR-β.

In some embodiments, the biomarker is a combination of biomarkers. In some embodiments, the combination of biomarkers comprises VEGFR1, VEGFR2, PDGFR-α and PDGFR-β. In some embodiments, the measuring the amount of the labelled hybrid polypeptide bound to the biomarker comprises a detection method. In some embodiments, the detection method is selected from the group consisting of Western Blot, immunoprecipitation, enzyme-linked immunosorbent assay (ELISA), immunohistochemistry, and radioimmunoassay. In some embodiments, the detection method is selected from the group consisting of spectroscopic, photochemical, biochemical, radiographical, immunochemical, chemical, electrical, and optical detection methods. In some embodiments, the detection method comprises detecting the concentration or the presence of the labelling agent. In some embodiments, the biological sample comprises tissue. In some embodiments, the biological sample comprises pterygium tissue. In some embodiments, the biological sample is in vivo or ex vivo.

The invention also provides methods for assessing a response of a subject to a therapy for treatment of an angiogenic disorder comprising: (a) contacting a first biological sample from the subject with a labelled hybrid polypeptide of the invention that binds to a biomarker and determining the amount of the biomarker in the first biological sample by measuring the amount of the labelled polypeptide bound to the biomarker; (b) contacting a second biological sample from the subject with the labelled polypeptide after the subject has been administered a therapeutic agent and determining the amount of the biomarker in the second biological sample by measuring the amount of the labelled polypeptide bound to the biomarker; and (c) determining whether the subject has a positive, negative, or neutral response to the therapy based on a comparison of the amounts of the biomarker in the first and second biological samples.

In some embodiments, the amount of the biomarker in a first biological sample is determined before treatment with a therapeutic agent, for example a therapeutic hybrid polypeptide of the invention. In some embodiments, the amount of the biomarker in a second biological sample is determined after completion of a treatment regimen with the therapeutic agent, for example 1 week, 2 weeks, 1 month, 2 months, or 6 months after completion of treatment regimen.

In some embodiments, determining the amount of biomarker in a sample or control comprises in vivo imaging, non-invasive or invasive. In some embodiments, determining the amount of biomarker in a sample or control comprises ex vivo imaging. In some embodiments, the biological sample is a biopsy sample or an aspiration sample.

The selection of a diagnostic control depends on the type of control (positive or negative), the type of biological sample, and whether the imaging is in vivo or ex vivo. For example, where the biological sample is an eye (for in vivo screening of an angiogenesis-related ocular disorder), in some embodiments, the negative control is the subject's healthy, non-affected eye. In some embodiments, the negative control is the average concentration of the biomarker present in a population of healthy, un-related, eyes where it is known that the subject does not suffer from any disease or condition that involves angiogenesis. For ex vivo determination of the biomarker concentration, such as ex vivo determination of the amount of the biomarker in a biopsy sample, is some embodiments, the control is a biopsy sample taken at an early date. In some embodiments, the control is subjected to the treatment as the biological sample.

In some embodiments the diagnostic absence, diagnostic presence, or change in the amount of a biomarker of an angiogenic disorder, for example an angiogenesis-associated disorder, is predictive of whether a therapy will be effective, or whether a therapy is having an effect. The individual may be treated with a hybrid polypeptide of the invention in accordance with the diagnosis.

Also provided are kits or articles of manufacture comprising the compositions described herein in suitable packaging. Suitable packaging for compositions (such as ophthalmic compositions) described herein are known in the art, and include, for example, vials (such as sealed vials), vessels, ampules, bottles, jars, flexible packaging (e.g., sealed Mylar or plastic bags), and the like. These articles of manufacture may further be sterilized and/or sealed.

The present invention also provides kits comprising compositions described herein and may further comprise instruction(s) on methods of using the composition, such as uses described herein. The kits described herein may further include other materials desirable from a commercial and user standpoint, including other buffers, diluents, filters, needles, syringes, and package inserts with instructions for performing any methods described herein.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible. In the following, examples will be described to illustrate parts of the invention.

### EXAMPLES

### Example 1

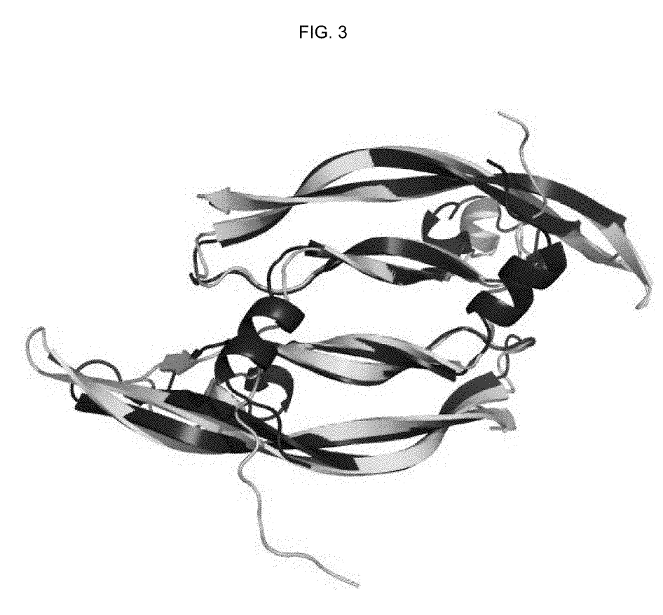

PDGF and VEGF have two binding epitopes which allow them to bind and dimerize two molecules of PDGFR and VEGFR, respectively, promoting activation and downstream cell signaling (FIG. 2A). To create antagonists towards PDGFR and VEGFR, we engineered a molecule that has only one binding site to PDGFR and VEGFR and that prevents receptor dimerization and activation. A comparison of the VEGF-A and PDGF-B crystal structures indicate significant overall structural homology (FIG. 3). Hence, we used a novel approach of engineering a hybrid PDGF/VEGF with PDGF residues on one pole and VEGF residues on the opposite pole that can be used to target both PDGFR and VEGFR (FIG. 2B). Despite the high structural similarity between VEGF-A and PDGF-B (RMSD 1.7 Å), the two growth factors have highly variable primary sequences, with only 21.6% sequence identity. Additionally, the respective receptor binding interfaces of VEGF and PDGF spans both chains of the growth factor dimers.

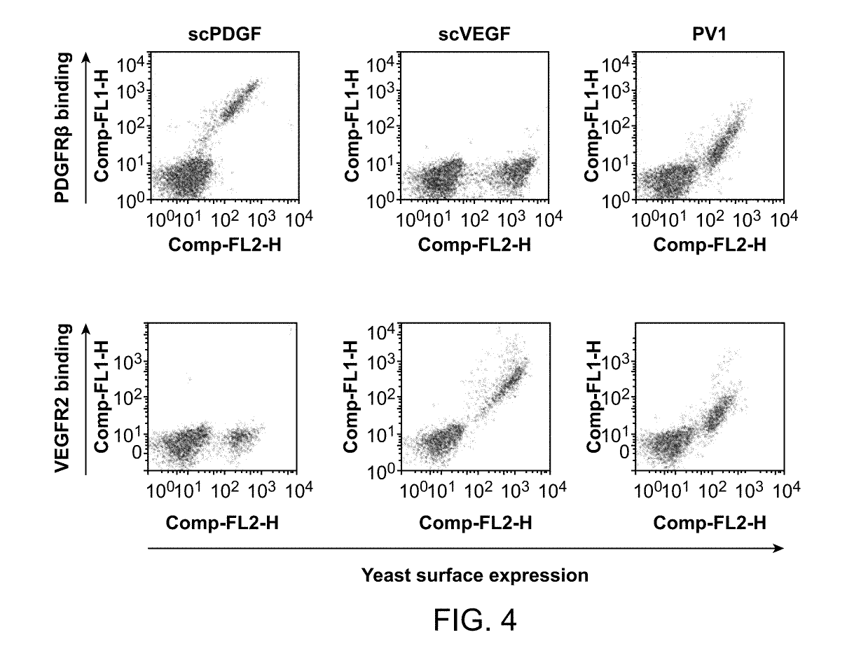

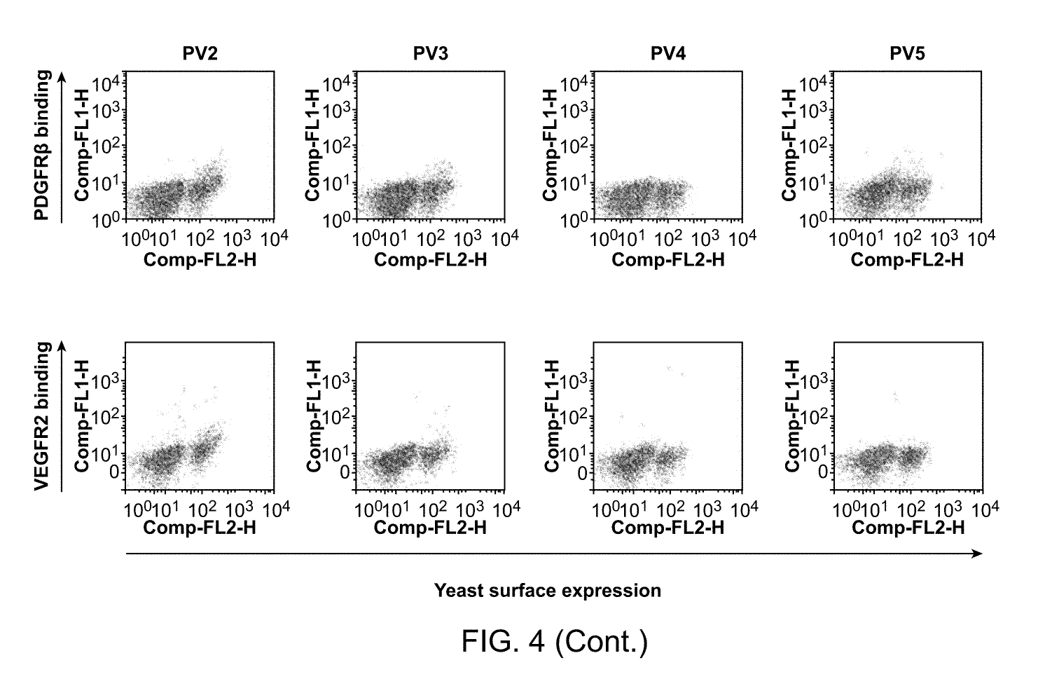

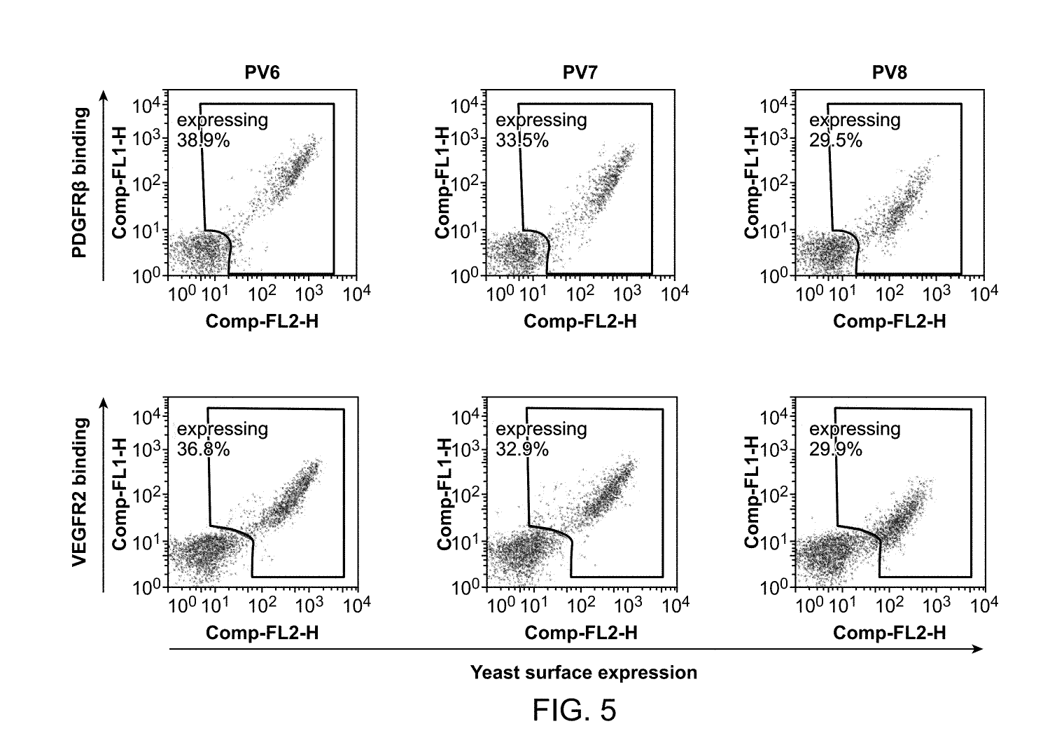

Since the receptor binding interface is composed of noncontiguous segments, a hybrid polypeptide was created by alternating PDGF and VEGF patches to preserve the PDGF-PDGFR and VEGF-VEGFR binding interfaces. Using molecular modeling, we tested various breakpoints at regions of structural overlap and/or conserved residues to determine which hybrid proteins can fold properly and adopt the native PDGF and VEGF structures at each pole. A 14-aa linker for creating a single-chain version of VEGF (scVEGF) was used to join the two chains of the dimer (Pantoliano et al. (1991), Biochemistry 30, pp. 10117-10125; Boesen et al. (2002), J Biol Chem 277(43), pp. 40335-40341). Each chain of the dimer required 4 breakpoints, resulting in 8 junctions total to create the hybrid protein. Genes for these constructs were synthesized, followed by site-directed mutagenesis for additional variants. Yeast display, where proteins of interest are presented as tethered fusions on the yeast cell surface, was used to test five initial hybrid designs for expression levels and for their ability to bind to both PDGFRβ and VEGFR2. All five constructs were able to be expressed on the yeast cell surface; however, only one construct (PV1) was functionally competent to bind to soluble PDGFRβ and VEGFR2 (FIG. 4). With the information gained from these initial constructs, we created three second-generation constructs using alternative breakpoints; all three bound to both PDGFRβ and VEGFR2 (FIG. 5), and in some cases (e.g., construct PV6) had higher expression and binding compared to PV1, and thus was used as a starting point for further engineering. This demonstrates that our strategy of creating a hybrid protein composed of 11 distinct segments was successful in creating a properly folded protein that retains the native PDGF-PDGFR and VEGF-VEGFR binding interfaces of the parent proteins.

Expression and characterization of PV6. The PDGF/VEGF hybrid polypeptide with N-terminal FLAG (DYKDDDDK) and C-terminal hexahistidine tags was solubly expressed in S. cerevisiae strain YVH10, and purified with metal chelating chromatography using Ni-NTA agarose resin followed by gel filtration chromatography. The protein was then characterized for binding affinity and inhibition of PDGF-induced PDGFR phosphorylation.

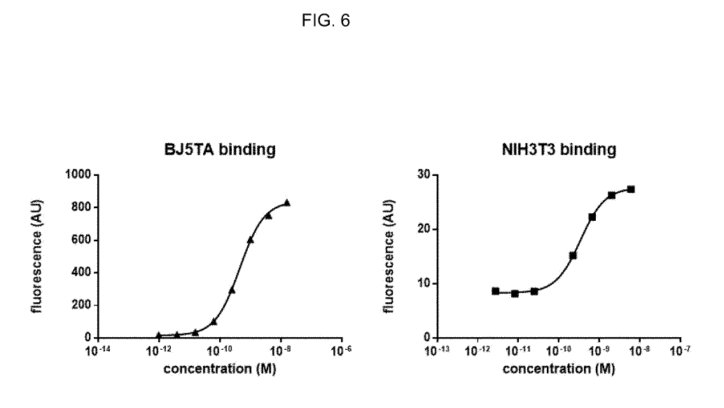

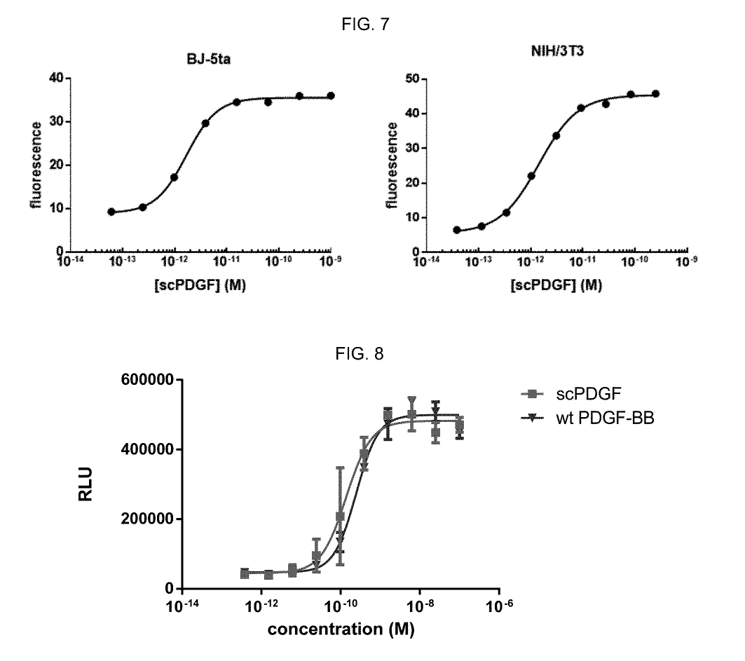

Binding affinity to PDGFR expressing cell lines. A binding titration of soluble PV6 was performed on BJ-5ta and NIH/3T3, human and mouse fibroblast cells that express PDGFR. We chose the NIH/3T3 cells for our initial studies because this is a commonly used cell line for the study of PDGF signaling, and BJ-5ta are human fibroblast cells that express human PDGFR. Cells were incubated with various concentrations of PV6 for 3 hours at 4° C. Binding was detected using an R-PE-conjugated anti-FLAG antibody, then analyzed by flow cytometry. We obtained K_{d} values of 0.45 nM and 0.34 nM for binding to BJ-5ta and NIH/3T3, respectively (FIG. 6). While this is a strong binding interaction, PV6 binds much weaker than a single-chain version of wild-type PDGF-BB (scPDGF), which has K_{d} values of 1.5±0.2 pM and 2.5±1.5 pM to BJ-5ta and NIH/3T3, respectively (FIG. 7). This was expected based on our design; our engineered molecule only has one binding site (which confers antagonistic activity) compared to the bivalent PDGF, which can bind with a much stronger apparent affinity to PDGFR on cells due to avidity effects.

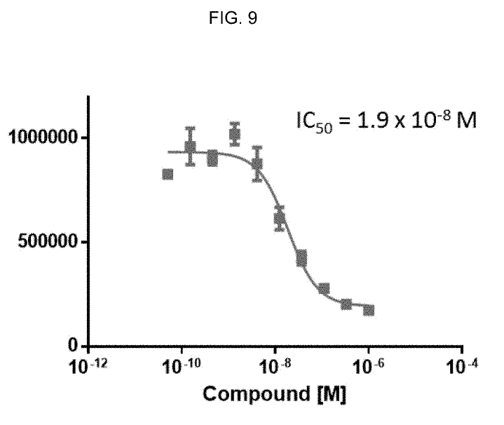

Phosphorylation of PDGFR. To determine PDGFR phosphorylation, we used the PathHunter Receptor Tyrosine Kinase reporter cell line developed by DiscoveRx, which allows for a cell-based functional assay to detect activated receptor tyrosine kinases. We tested the ability of scPDGF to activate the DiscoveRx PathHunter PDGFR cell line. As shown in FIG. 8, we observed a robust signal and broad dynamic range in the dose response curves, with an EC₅₀ of 0.14 nM and 0.25 nM for scPDGF and wt PDGF-BB, respectively. The response of scPDGF and wt PDGF-BB are very similar, demonstrating that scPDGF behaves similarly to wt. We then tested whether PV6 can inhibit PDGF-induced phosphorylation of PDGFR. We found that PV6 can completely inhibit stimulation by 0.1 nM scPDGF with an IC₅₀ of 19 nM (FIG. 9). This demonstrates that our antagonist behaves as expected, that a molecule with only one binding site for PDGF will bind to and inhibit PDGFR activation.

### Example 2

The rationally-engineered hybrid polypeptide is expected to bind only one copy of PDGFR or VEGFR, (a design feature that confers antagonistic activity); thus, it will have weaker affinity compared to the native bivalent PDGF and VEGF proteins. It is desirable for a competitive antagonist to be able to effectively outcompete the strong affinity of endogenous growth factor (10 pM and 0.5 nM for PDFGR and VEGFR, respectively). Otherwise, high dosing is required to achieve therapeutic benefit. The PDGF/VEGF hybrid was thus engineered to have high affinity binding to both PDGFR and VEGFR.

Generation of yeast-displayed library: We used combinatorial methods to engineer the hybrid PDGF/VEGF to have increased binding affinity and a slower kinetic off-rate of binding to PDGFR or VEGFR. Error-prone PCR was used to create a library of 6×10⁷ million variants that was displayed on the yeast cell surface.

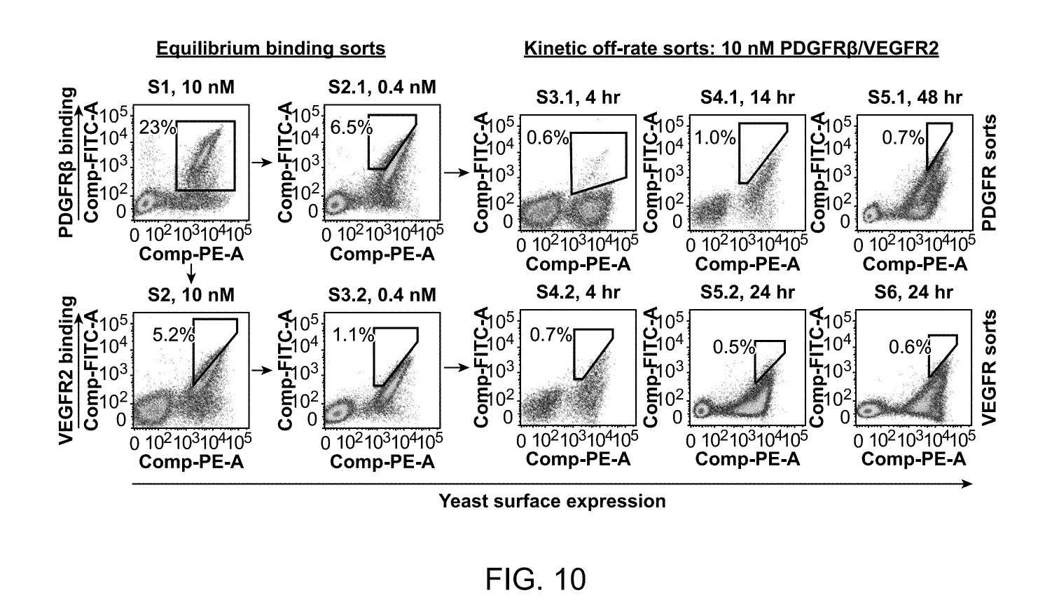

Library screening with soluble PDGFR and VEGFR: Five or six consecutive rounds of flow cytometric sorting were used to isolate yeast-displayed hybrid PDGF/VEGF that bound to higher levels of soluble PDGFR or VEGFR extracellular domains, for a given amount of expression, as detected by fluorescent antibodies. Two parallel library screens were performed, one with soluble PDGFRβ and one with soluble VEGFR2, to separately identify mutations that conferred high affinity binding to PDGFR or VEGFR, respectively. To differentiate the highest affinity clones, the concentration of soluble receptor was decreased with each round of sorting to increase the screening stringency. In addition, for the last few rounds of library screening, “off-rate” screens were performed in which binding reactions were sorted 4 to 48 hours post removal of unbound receptor to isolate hybrid PDGF/VEGF variants with decreased kinetic off-rates that retained binding to PDGFR or VEGFR (FIG. 10). Sequencing of individual clones isolated from these library screens identified mutations that conferred increased binding affinity to PDGFRβ(˜500-fold decrease in K_{d} compared to the starting protein) or VEGFR2 (>100-fold decrease, the K_{d} of the starting protein is too weak to accurately determine in this assay) as measured on the surface of yeast. Importantly, the variants bind with increased affinity to both mouse and human receptors.

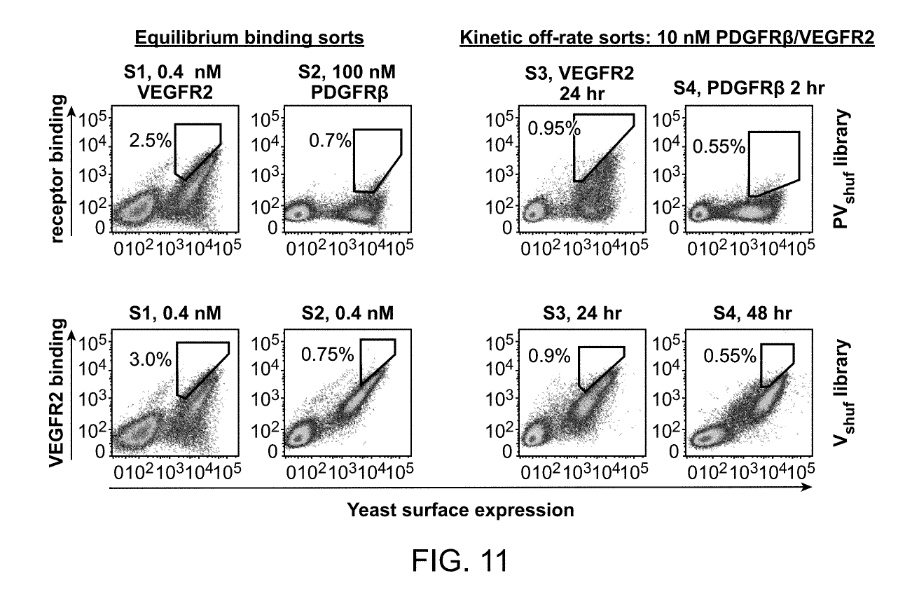

Engineered hybrid PDGF/VEGF with increased binding affinity to both PDGFR and VEGFR2: We created a variant that has increased binding affinity to both PDGFR and VEGFR2 by combining the mutations that we found for increased affinity to each receptor. We used in vitro recombination to combine the beneficial mutations and remove silent and deleterious mutations. This process was employed using the staggered extension process (StEP) developed by Zhao and Arnold (2006) Nat. Protoc. 1, 755-68. We performed a second StEP reaction using only the sequences obtained from the VEGFR2 sorts. For the recombined PDGFR and VEGFR sequences (PV_shuf), we performed two rounds of equilibrium sorts followed by two rounds of “off-rate” sorts (FIG. 11), alternating between PDGFR and VEGFR binding for each sort. For the recombined VEGFR sequences (V_shuf), we performed two rounds of equilibrium sorts followed by two rounds of “off-rate” sorts for VEGFR binding.

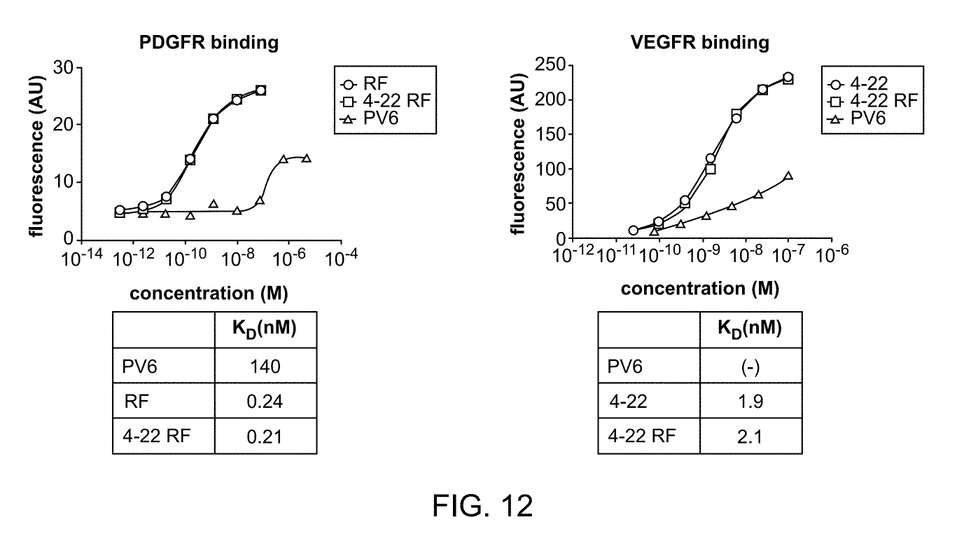

Since the mutations for PDGFR and VEGFR binding appeared to be additive, we combined the two mutations for PDGFR binding with the four mutations for VEGFR binding to create a variant that has high affinity to both PDGFR and VEGFR (named 4-22 RF). This variant has almost identical binding to PDGFR and VEGFR as the PDGFR and VEGFR only clones, with K_{d} of 0.21 nM to PDGFR and 2.1 nM to VEGFR (FIG. 12).

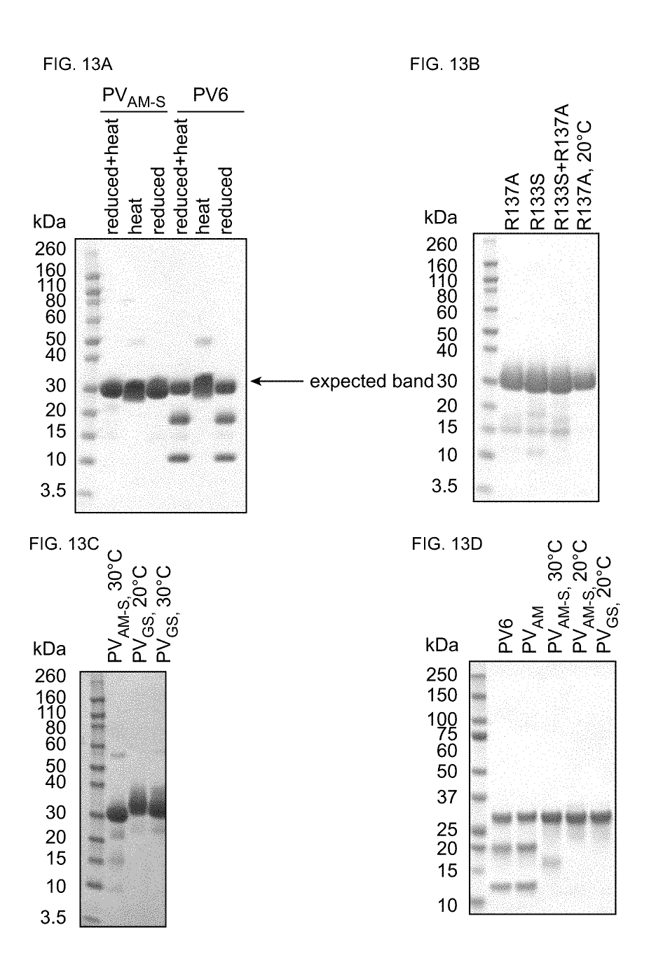

Expression and characterization of engineered hybrids: The hybrid PDGF/VEGF was solubly expressed and purified as described above. While performing soluble expression of our protein, we noticed there were extra bands in our final product, which was indicative of protein degradation. Previous studies have shown that PDGF has a protease site at arginine-32 (Cook et al. (1992), Biochem. J. 281, pp. 57-65). We mutated arginine-30 in HC2 (arginine-32 in PDGF and arginine-137 in PV6) to alanine in our final construct (named PV R32A), and found that degradation was significantly eliminated upon expression of this protein at 20° C. (FIG. 13).

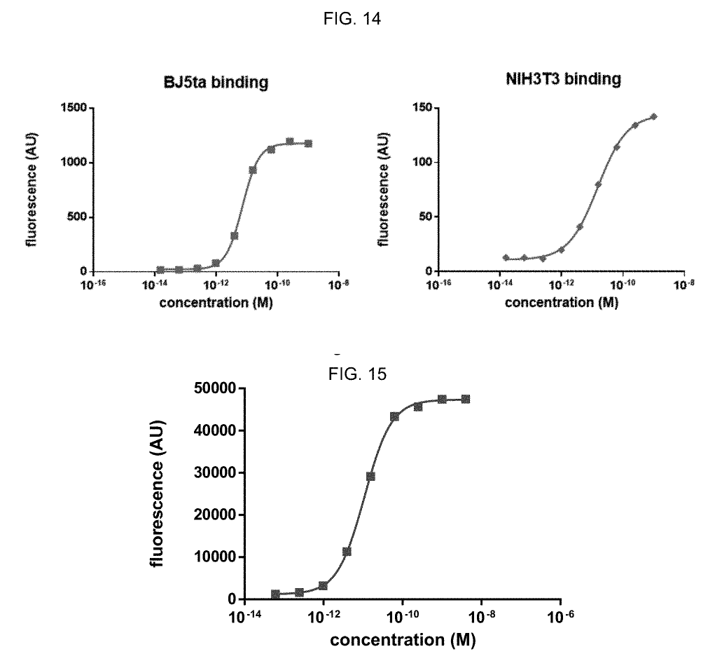

Binding affinity to PDGFR and VEGFR expressing cell lines: A binding titration of soluble PV RF (PV K87R chain 1 and PV I182F chain 2, obtained from PDGFR sorts) and PV R32A (final affinity-matured variant) was performed on BJ-5ta and NIH/3T3 mouse fibroblast cells that express PDGFR, using our protocol described previously. Cells were incubated with various concentrations of PV RF or PV R32A for 3 hours at 4° C. Binding was detected using an R-PE-conjugated anti-FLAG antibody, then analyzed by flow cytometry. We obtained K_{d} values of 7.1 pM and 15 pM for PV RF binding to BJ-5ta and NIH/3T3, respectively (FIGS. 14) and 10.9 pM for PV R32A binding to BJ-5ta (FIG. 15). This represents a decrease of 63-, 22-, and 41-fold in K_{d} compared to PV6. This is comparable but not quite as strong binding compared to the bivalent scPDGF, which has K_{d} of 1.5 pM and 2.5 pM to BJ-5ta and NIH/3T3, respectively (FIG. 7). However, our values are similar to the reported literature value of 10 pM.

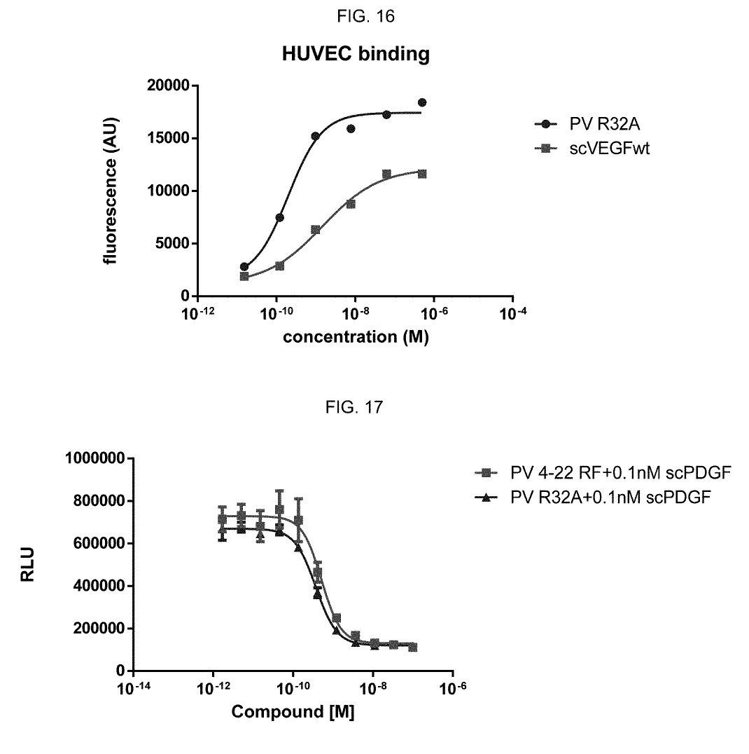

A binding titration of soluble PV R32A was performed on human umbilical vein endothelial cells (HUVECs) that express VEGFR. Cells were incubated with various concentrations of hybrid PDGF/VEGF for 3 hours at 4° C. Binding was detected using an R-PE-conjugated anti-FLAG antibody, then analyzed by flow cytometry. We obtained K_{d} values of 0.2 nM for PV R32A and 1.5 nM for a single-chain version of VEGF (scVEGFwt) (FIG. 16). This represents a 7.5-fold improvement over the bivalent scVEGFwt.

Collectively, these results demonstrate that we have successfully engineered a protein that acts as a potent antagonist for both PDGFR and VEGFR activation, in accordance with our design principles.

### Methods

Engineering PDGF/VEGF hybrid constructs. We used the structural homology of VEGF-A and PDGF-B to engineer a hybrid protein with PDGF residues on one pole and VEGF residues on the opposite pole that can bind to both PDGFR and VEGFR. Using molecular visualization in PyMOL, various breakpoints were introduced at regions of structural overlap and/or conserved residues to create the PDGF/VEGF hybrid polypeptide. Gene constructs for these variants were synthesized using custom gene synthesis (Biomatik Corporation, Life Technologies). The genes were cloned into yeast display vector pCT with restriction sites Nhel and Mlul. Mutations for making additional PDGF/VEGF variants were created by oligonucleotide site-directed mutagenesis.

Library creation. PV6 was used as the initial template for error-prone PCR. Mutations were introduced using low fidelity Taq polymerase (New England Biolabs) and nucleotide analogs 8-oxo-dGTP and dPTP (TriLink Biotech). To obtain a range of mutation frequencies, six PCRs were performed with varying concentrations of nucleotide analogs and number of PCR cycles: 5 cycles (200 μM analogs), 10 cycles (2 μM, 20 μM, 200 μM), and 20 cycles (2 μM, 20 μM). Primers consisting of 50 bp overlap with the pCT plasmid in the forward and reverse direction were designed for homologous recombination in yeast. PCR products were amplified in the absence of nucleotide analogs and purified on a 1% agarose gel. Five transformations of 5 ug purified DNA and 1 ug restriction enzyme digested pCT were electroporated into EBY100 competent yeast. A library size of 6.1×10⁷ transformants was obtained, estimated by dilution plating.

Staggered extension process (StEP) was used to perform in vitro recombination of our error-prone PCR library sort products. For the PV_shuf library, templates from the PDGFR sort 4 (22.5%) and 5 (22.5%) were mixed with VEGFR sort 6 (45%) and PV6 (10%). For V_shuf library, templates from VEGFR sort 6 (90%) and PV6 (10%) were mixed. PCR was performed using Taq polymerase with short cycles of 5 s extension. Thermal cycling conditions: 1 cycle at 94° C. for 30s; 60 cycles of 94° C. for 30s, 55° C. for 5s; 4° C. for ∞. PCR products were amplified and purified on a 1% agarose gel. 5 ug purified DNA and 1 ug restriction enzyme digested pCT were electroporated into EBY100 competent yeast. A library size of 5.1×10⁶ and 1.4×10⁷ transformants was obtained for PV_shuf and V_shuf, respectively, estimated by dilution plating.

Library screening. Yeast displaying hybrid PDGF/VEGF variants with high affinity PDGFR or VEGFR binding were isolated by fluorescence-activated cell sorting (FACS). For equilibrium sorting, various concentrations of soluble PDGRβ-Fc or PDGRβ-His (expressed and purified in Cochran Lab) or VEGFR2-Fc (R&D Systems) were incubated with yeast-displayed libraries in phosphate-buffered saline with 1 mg/ml BSA (PBSA) at room temperature. Cells were incubated in large enough volumes to avoid ligand depletion and appropriate time to reach equilibrium. During the last hour of incubation, yeast were incubated with 1:250 dilution of chicken anti-c-Myc (Invitrogen) in PBSA. Yeast were then washed and pelleted, and incubated with 1:100 dilution of secondary antibodies for 10 minutes on ice: anti-mouse-IgG (Fc specific)-AF488 (Invitrogen) for PDGRβ-Fc, anti-His Hilyte Fluor 488 (Anaspec) for PDGRβ-His, anti-human-IgG (Fc specific)-FITC (Sigma) for VEGFR2-Fc, and phycoerythrin-conjugated goat anti-chicken-IgY-PE (Santa Cruz Biotechnology). Yeast were then washed and pelleted, and resuspended in PBSA right before FACS sorting.

For kinetic off-rate sorts, yeast were incubated with of soluble 10 nM PDGRβ-Fc or PDGRβ-His or VEGFR2-Fc for 1 hr at room temperature, then washed with PBSA to remove unbound receptor. Cells were then resuspended in 100 nM scPDGF or scVEGFwt (expressed and purified in Cochran Lab), and incubated for various times so that any receptor that dissociates from the yeast surface will not rebind. Yeast were incubated with primary and secondary antibodies as described above.

Labeled yeast were sorted by FACS using BD FACSAria II (Stanford FACS Core Facility). Sorted clones were amplified in SD-CAA pH 4.5 media, induced for expression at SG-CAA, and subjected to further rounds of labeling and FACS. Sorting stringency was increased by lowering the concentration of PDGFR or VEGFR for equilibrium sorts and increasing the time for dissociation for kinetic off-rate sorts. Plasmid DNA was recovered from sorted yeast using a Zymoprep kit (Zymo Research Corp.), transformed into XL-1 blue supercompetent cells (Stratagene), and isolated using plasmid miniprep kit (Qiagen). Sequencing was performed by Sequetech Corp.

Yeast surface display and binding. Plasmids were transformed into Saccharomyces cerevisiae strain EBY100 by electroporation. Yeast were grown in selective media and induced for expression and display on the cell surface in galactose media according to established protocols. 50,000 yeast cells were incubated with PDGRβ-Fc, PDGFR-His, or VEGFR2-Fc, followed by labeling with primary and secondary antibodies as described for the library sorts. Cells were analyzed by flow cytometry using a FACSCalibur instrument (Becton Dickinson), and data was quantified using FlowJo software (Treestar).

Recombinant expression and purification of scPDGF and hybrid PDGF/VEGF. scPDGF and hybrid PDGF/VEGF constructs were cloned into a vector containing N-terminal FLAG and C-terminal hexahistadine tags and transformed into Saccharomyces cerevisiae strain YVH10 using electroporation. Transformed yeast were selected on SD-SCAA plates with Leu2 as a selectable marker. Cells were inoculated into liquid SD-SCAA and induced in galactose containing SG-SCAA media. Cells were harvested after 3 days, and the supernatants were collected for protein purification. Proteins were purified using metal chelating chromatography. Briefly, proteins were bound to Ni-NTA agarose resin (Invitrogen), washed with binding buffer (50 mM NaH₂PO₄, 300 mM NaCl, 10 mM imidazole, pH 8), and eluted with elution buffer containing 250 mM imidazole. N-linked glycosylation was removed by digestion with Endo Hf (New England Biolabs), and proteins were further purified by size exclusion chromatography on a Varian ProStar HPLC system, using a Superdex 75 column (GE Healthcare) and PBS running buffer. Purified proteins were concentrated using an Amicon Ultra-0.5 mL centrifugation filter (Millipore) with a 10 kDa molecular weight cutoff membrane.

Recombinant expression and purification of PDGFRβ. Residues 1-314 encoding the first three Ig-like domains of PDGFR-β was cloned from the cDNA (Genbank Accession ID BC032224) into pAdd2 vector for HEK293 cell expression. For the PDGRβ-His construct, residues 1-314 were attached to a C-terminal hexahistadine tag and cloned into pAdd2 using EcoRl and Xhol restriction sites. For PDGRβ-Fc, residues 1-314 were fused to the Fc region of mouse IgG2a and cloned into pAdd2 using EcoRl and Sall. DNA was transfected into FreeStyle 293-F cells according to the manufacturer's protocols for the FreeStyle MAX 293 Expression System (Invitrogen). Transfection was performed with the cationic lipid-based transfection reagent, FreeStyle MAX Reagent. For a 30 ml culture, 37.5 μl of 1mg/m1 plasmid DNA was added to 600 μl of OptiPRO SFM, and 37.5 μl of FreeStyle MAX Reagent was added to 600 μl of OptiPRO SFM. The diluted FreeStyle MAX Reagent solution was mixed with the diluted DNA solution and incubated for 10-20 minutes at room temperature for DNA-lipid complexes to form. The DNA-lipid mixture was then slowly added to cell culture while swirling the flask. Transfected cells were incubated at 37° C., 8% CO2 on an orbital shaker rotating at 135 rpm. Cells were harvested after 6 days, and the supernatants were collected for protein purification.

PDGRβ-His was purified using metal chelating chromatography followed by size exclusion chromatography as described above. PDGRβ-Fc was purified using Protein A chromatography followed by size exclusion chromatography. Briefly, PDGRβ-Fc was bound to Protein A-Sepharose (Invitrogen), washed with binding buffer (0.02 M NaH₂PO₄ and 0.15 M NaCl, pH 8), and eluted with pH 5 elution buffer (0.2 M Na₂HPO₄ and 0.1 M citric acid). Proteins were eluted into neutralization buffer (1 M Tris-HCl, pH 8.8) to a pH of 7.4. Proteins were further purified by size exclusion chromatography using a Superdex 200 column (GE Healthcare) and concentrated as described above.

PDGFR phosphorylation assay. BJ-5ta cells were grown in 4 parts Dulbecco's Modified Eagle Medium (Gibco 11995) and 1 part M199 (Gibco 11150), supplemented with 10% fetal bovine serum (Gibco 10437) and 0.01 mg/ml hygromycin B (Gibco 10687). NIH/3T3 cells were grown in Dulbecco's Modified Eagle Medium supplemented with 10% calf serum (Gibco 16010). BJ-5ta or NIH/3T3 cells were seeded on 6-well plates at a density of 25,000 cells/well. After 24 hr, cells were replaced with serum free media (SFM). After 24 hr of serum starvation, cells were stimulated with 1 ml of varying concentrations of PDGF-BB (Peprotech) and hybrid PDGF/VEGF in SFM+0.1% BSA for 10 min at 37° C. in the presence of phosphatase inhibitor cocktail 2 (Sigma). Cells were washed with ice-cold PBS and treated with 100 μl NP-40 lysis buffer (20 mM Tris-HCl, pH 8.0, 137 mM NaCl, 10% Glycerol, 1% Nonidet P-40) with 1× phosphatase inhibitor cocktail 2 and 1× protease inhibitor cocktail 2 (Sigma) for 1 hr at 4° C. Lysates were frozen at −80° C., clarified by centrifugation, and quantified with BCA assay (Pierce). Lysates were then separated on a NuPAGE Novex 4-12% Bis-Tris gel (Invitrogen) with MES buffer under reducing conditions, then transferred to a nitrocellulose membrane using the iBlot module (Invitrogen). Immunoblots were incubated in 5% milk in Tris-buffered saline with 1 mg/L Tween 20 (TBST), probed with primary antibodies overnight at 4° C., washed with TBST, then probed with secondary antibodies for 1 hr at room temperature. Antibodies were incubated in 5% milk in TBST. Primary antibodies used were anti-phospho-PDGFRβ (Cell Signaling 2227) at 1:1000 dilution and anti-β-tubulin (Covance MMS-410P) at 1:10,000 dilution. Secondary antibodies conjugated with horseradish peroxidase used were anti-rabbit (Jackson Immunoresearch 711-035-152) at 1:5000 dilution for anti-phospho-PDGFRβ and 1:50,000 for anti-β-tubulin. After secondary antibody incubation, immunoblots were developed using SuperSignal West Femto (Thermo), and chemiluminescence was detected on a ChemiDoc XRS+System (Bio-Rad).

VEGFR phosphorylation assay. HUVECs were seeded on 6-well plates at a density of 100,000 cells/well in EGM BulletKit Media (Lonza CC-3124). After 24 hr, cells were replaced with serum-starvation media (SSM): EBM (Lonza CC-3121) with 0.2% BSA and 0.5% FBS. After 12 hr of serum starvation, cells were stimulated with 1 ml of varying concentrations of VEGF₁₆₅ (Peprotech) and hybrid PDGF/VEGF in SSM for 8 min at 37° C. in the presence of phosphatase inhibitor cocktail 2. Cells were washed with ice-cold HEPES-BSS (Lonza CC-5024) and treated with 100 μl NP-40 lysis buffer with 1× phosphatase inhibitor cocktail 2 and 1× protease inhibitor cocktail 2 for 1 hr at 4° C. Immunoblotting of cell lysates were performed as described above. Primary antibodies used were anti-phospho-VEGFR2 Y1175 (Cell Signaling 2478) at 1:1000 dilution and anti-p-tubulin (Covance MMS-410P) at 1:10,000 dilution. Secondary antibodies conjugated with horseradish peroxidase used were anti-rabbit (Jackson Immunoresearch 711-035-152) at 1:5000 dilution for anti-phospho-VEGFR2 and 1:50,000 for anti-β-tubulin.

DiscoveRx PathHunter cell-based kinase assay. PathHunter U2OS PDGFRb cells (DiscoveRx) were cultured in PathHunter select U2OS cell culture media K (DiscoveRx). The PathHunter cell-based assay was performed according to DiscoveRx protocols. Cells were plated in PathHunter Cell Plating 16 media on 384-well plates at a density of 10,000 cells/well. After 24 hr, cells were stimulated with various concentrations of PDGF ligands for 3 hr at room temperature. PathHunter Detection Reagent (DiscoveRx) was added and incubated for 1 hr at room temperature to detect activated receptor. Chemiluminescence signal was analyzed on a microplate reader (BioTek).

Cell-binding assay. Varying concentrations of scPDGF, scVEGF, and PDGF/VEGF ligands were incubated with 25,000 cells (BJ-5ta, NIH/3T3, or HUVEC) in binding buffer (20 mM Tris pH 7.5 with 1 mM MgCl₂, 1 mM MnCl₂, 2 mM CaCl₂, 100 mM NaCl, and 1 mg/ml BSA) for 3 hr at 4° C. Cells were washed with binding buffer and incubated with 1:100 R-PE-conjugated anti-FLAG antibody (Prozyme) for 15 min on ice. Cells were washed with binding buffer and analyzed by flow cytometry using a guava easyCyte (Millipore) or BD Accuri, and data was quantified using FlowJo software (Treestar).

### Example 3

### Engineering Chimeric PDGF/VEGF that Binds and Antagonizes both PDGFR and VEGFR

PDGF-B and VEGF-A are part of the PDGF family of cystine knot growth factors. They share the same overall fold, and comparison of the two crystal structures show significant structural homology, with RMSD of 1.7 Å (FIG. 3). PDGF and VEGF have two binding epitopes that allow them to bind to two PDGFR and two VEGFR, respectively, leading to dimerization of the receptors and activation of downstream signaling. We used the structural similarity and the bivalent nature of the two growth factors to engineer a chimeric PDGF/VEGF with PDGF on one pole and VEGF on the opposite pole that can be used to target both PDGFR and VEGFR and antagonize both receptors (FIG. 2).

Despite the structural similarity between PDGF-B and VEGF-A, the two growth factors have highly variable primary sequences, with 21.6% sequence identity. Additionally, each growth factor is a disulfide-bonded dimer, and the receptor binding interface is composed of noncontiguous segments that span both chains of the dimer. Hence, the PDGF/VEGF chimera was created by alternating PDGF and VEGF patches to preserve the native PDGF-PDGFR and VEGF-VEGFR binding interfaces. To facilitate the correct heterodimer formation in the chimera, a 14-aa linker was used to join the two chimeric PDGF/VEGF chains, derived from the linker used for creating a single-chain version of VEGF.

Using molecular modeling, we proposed various crossover points at regions of structural overlap and/or conserved residues to determine which chimeric proteins can fold properly and adopt the native PDGF and VEGF structures at each pole. Each chain of the dimer required 4 crossover points, resulting in 8 junctions total to create the chimera (FIG. 1). Yeast surface display, where proteins of interest are presented as tethered fusions on the yeast cell surface, was used to test five initial chimera designs for expression levels and for their ability to bind to both PDGFRβ and VEGFR2. Yeast display allows us to quickly express and test variants without purification, and presents the proteins in the correct format for library generation and screening, as discussed in the next section. Out of five initial constructs, all were expressed well on the yeast cell surface; however, only one construct (PV1) was functionally competent to bind to soluble PDGFRβ and VEGFR2 (FIG. 4). PV1 differs from the other four constructs at only one crossover point. The difference at that crossover may have avoided a potential steric clash between VEGF E30 and PDGF E45.

With the information gained from the initial constructs, we created three additional constructs using alternative crossover points. At each crossover, care was taken to ensure that each new PDGF-VEGF junction minimizes the disruption of any side-chain interactions of the parent molecules. For example, analysis of the crystal structure of PDGF reveals that S50 interacts with K17 and R19 and Q59 with R61. Similarly, VEGF E103 interacts with R105. All three new designs bound to both PDGFR and VEGFR2 (FIG. 5). PV6 had the highest expression and binding, and thus was used as a starting point for further engineering.

Engineering chimeric PDGFNEGF variants with increased binding affinity to PDGFR and VEGFR. The rationally-engineered PDGFNEGF chimera is expected to bind only one copy of PDGFR or VEGFR (a design feature that confers antagonistic activity); thus, it will have weaker affinity for binding to cell surface receptors compared to the native bivalent PDGF and VEGF due to loss of avidity. In order for a competitive antagonist to be a viable therapeutic candidate, it must be able to effectively outcompete the strong affinity of endogenous growth factor (10 pM and 0.5 nM for PDFGR and VEGFR, respectively). Otherwise, high dosing is required to achieve therapeutic benefit, which is not cost effective and introduces toxicity and off-target effects. The PDGFNEGF chimera was thus engineered to have high affinity binding to both PDGFR and VEGFR. Two rounds of directed evolution were used to engineer PV6 to have increased binding affinity to PDGFR or VEGFR.

Random mutagenesis library generation. For the first round of directed evolution, error-prone PCR was used to create a library that introduced random mutations throughout PV6. While the binding interface of PDGF-PDGFR and VEGF-VEGFR are known from crystal structures of the complexes, we did not want to perform saturation mutagenesis of the binding interface residues. Since we were starting with the strong binding affinity of the native PDGF-PDGFR and VEGF-VEGFR interactions, we wanted to only make a few small changes to improve the binding interaction without disrupting the strong native interaction. Inserting mutations throughout the protein would also allow us to identify mutations that affect the core of the protein, and is particularly beneficial for the PDGFNEGF chimera, as we may identify mutations that help stabilize and improve the chimera structure.

Random mutations were introduced into PV6 using error-prone PCR, and the DNA was transformed into yeast to create a library of 6.1×10⁷ variants that was displayed on the yeast cell surface. Sequence analysis of 20 clones revealed that there were 0-28 base pair mutations and 0-15 amino acid mutations, corresponding to a mutation rate of 0-4.5% and 0-7.2%, respectively. Yeast display allows us to efficiently screen and isolate variants with improved binding affinity and expression. Our protein of interest is displayed a fusion protein that is anchored to the yeast cell surface, along with a c-myc epitope tag that can be used to detect for full-length protein expression. To analyze for binding, the soluble protein partner (in our case, soluble PDGFR or VEGFR extracellular domains) was incubated with the yeast displayed proteins. Next, fluorescently-labeled antibodies were used to label the bound receptors and c-myc tag on the cells. The cells were then analyzed by flow cytometry. Preliminary analysis of the unsorted library shows significant binding to both PDGFR and VEGFR, at concentrations of 10 nM PDGRβ-Fc and 10 nM VEGFR2-Fc. A portion of the library members lost their ability to bind to PDGFR and VEGFR or had low expression, which is expected as many of the random mutations would be deleterious to the folding and/or binding of the protein.

Random mutagenesis library screening. Five or six consecutive rounds of fluorescence-activated cell sorting (FACS) were used to isolate yeast-displayed PDGF/VEGF chimeras that bound to higher levels of soluble PDGRβ-Fc or VEGFR2-Fc relative to protein expression. Since the random mutagenesis library may not contain enough mutations to improve the binding affinity to both PDGFR and VEGFR, two parallel library screens were performed, one with soluble PDGRβ-Fc and one with soluble VEGFR2-Fc, to separately identify mutations that conferred high affinity binding to each receptor.

In the initial round of sorting, the objective was to screen through as much of the library as possible to remove all the nonbinding members of the library (FIG. 10). In the second round of sorting, the screens were separated into PDGFR and VEGFR screens. High affinity binders were identified by the levels of equilibrium binding to a constant concentration of PDGRβ-Fc and VEGFR2-Fc. Concentrations were selected using a pre-sort analysis to give the best separation of high and low affinity binders. To differentiate the highest affinity clones, the concentration of soluble receptor was decreased with each round of sorting to increase the screening stringency. A diagonal sort gate was drawn to normalize the levels of PDGFR or VEGFR binding to the levels of full-length protein expression (detected by c-myc expression levels).

After 2 rounds of sorting for PDGFR and 3 rounds of sorting for VEGFR, it was increasingly difficult to separate the highest affinity clones by equilibrium binding. An equilibrium screen for concentrations below 0.4 nM would require excessively large volumes to avoid ligand depletion and long incubation times to reach equilibrium binding. Hence, an alternative screening strategy was used, based on the kinetics of the binding reaction. K_{d} is defined a ratio of the kinetic parameters k_{off}/kₒₙ. Differences in binding affinity are largely due to the dissociation rate k_{off}. Therefore, we used a kinetic off-rate sort to screen for variants that have the slowest off-rates.

For the “off-rate” screens, the library was first incubated with 10 nM of PDGRβ-Fc or VEGFR2-Fc and allowed to reach equilibrium. Cells were then washed to remove any unbound receptor. Without free ligand in solution, the binding reaction will shift towards dissociation of the bound receptor from the yeast surface, as the proteins approach a new equilibrium to the solution without any ligand. During the dissociation step, cells were incubated with high concentrations of soluble scPDGF or scVEGF as competitor, such that any receptor that dissociates will bind to the competitor, rendering the dissociation from the yeast surface irreversible. Cells were then screened after 4 to 48 hours of dissociation to sort for cells that retained the highest binding, indicating slower off-rate. The dissociation times were increased from 4 hours in round 3 to 48 hours for round 5 in the PDGFR sorts and 4 hours in round 4 to 24 hours for round 6 in the VEGFR sorts (FIG. 10). In addition, sort gates were drawn to be more stringent in the latest rounds to only collect the top 0.5-1% of the highest binding and expressing populations.

Post-sort analysis of the sorted cells reveals that the library was enriched for better binders in both equilibrium binding and slower dissociation as the sorts progressed. The final sort products retained high levels of binding after 48 hours of dissociation for PDGFR and 24 hours of dissociation for VEGFR, while the original library were mostly dissociated.

Analysis of random mutagenesis library sort products. After 4 rounds of sorting, sequences of individual clones were analyzed to determine whether the library had sufficiently reached consensus mutations. For the PDGFR sorts, two consensus mutations emerged. After round 4, K87R was the dominant mutation, occurring in 13 out of 20 clones; after round 5, 1182F was the dominant mutation that occurred in all 9 clones, with 7 repeats of the clone containing only that mutation. For the VEGFR sorts, several mutations occurred multiple times, such as F2OL, F20S, Q71R, and repeats T90A, G100D, T123A, D160H in the sort 5 products. An additional round 6 was performed, resulting in repeats of T90A, G100D, T123A, D160H and M6V, F20L, E28G, Q71R, G100D, and S103P. The high frequency of repeated sequences in round 5 of the PDGFR sorts and round 6 of the VEGFR sorts indicated that the sorts were sufficiently enriched and further sorts would not be productive. However, with the large number of mutations identified in the VEGFR sorts, we continued with a second library screen to determine which of the mutations were most beneficial for binding.

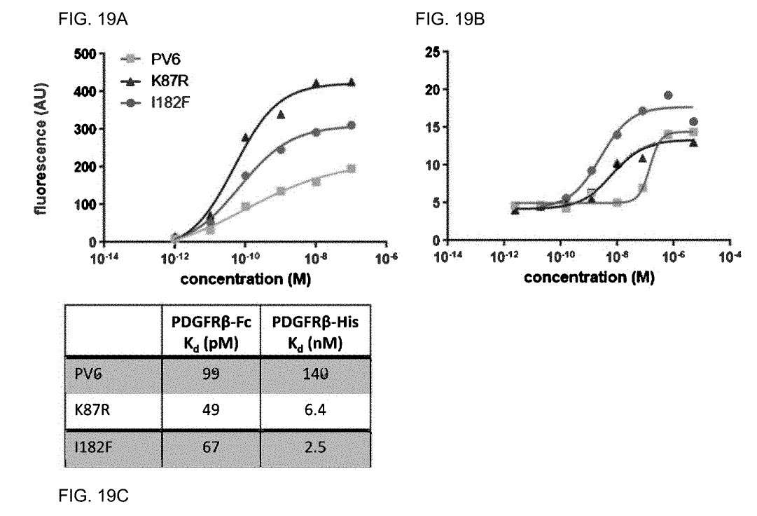

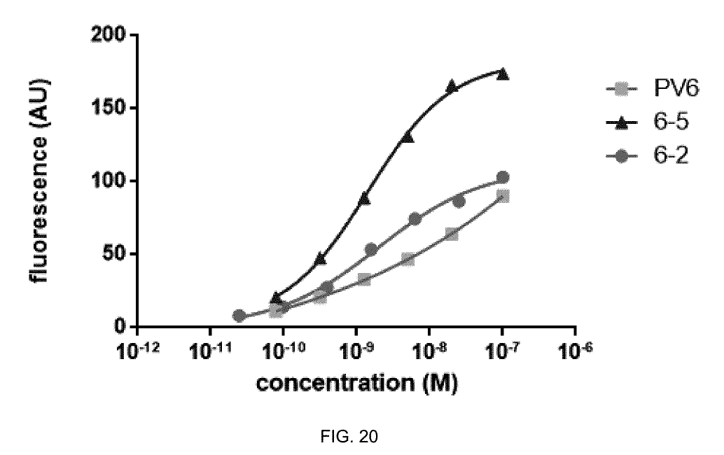

PDGRβ-Fc has an extremely strong binding affinity to PDGF (˜40 pM), which made it difficult to distinguish between the affinities of tight binders. When compared to PV6, it is not apparent that the mutations K87R and I182F showed improvements in binding affinity to PDGRβ-Fc, but the maximum levels of binding are higher (FIG. 19A, C). Binding of PDGRβ-Fc to PV6 had a K_{d} of 99 pM, while K87R and I182F had K_{d} of 49 and 67 pM, respectively. We tested the monovalent version of PDGFR (PDGRβ-His) to remove any avidity effects that may affect the measurements. This version of the receptor showed much weaker binding affinity to yeast displayed PDGF, and reveals the improvement in affinity for the PDGFR sort products (FIG. 19B, C). PV6 bound to PDGRβ-His with a K_{d} of 140 nM, while K87R and I182F had K_{d} of 6.4 and 2.5 nM, respectively. For VEGFR2-Fc, binding affinity of the two dominant clones in sort 6, 6-2 and 6-5, were compared to PV6 (FIG. 20). PV6 bound to VEGFR too weakly to accurately determine a K_{d}, while 6-2 and 6-5 had a K_{d} of 2.0 and 1.4 nM, respectively.

In vitro recombination library generation and screening. To create a PDGF/VEGF chimera with increased affinity to both PDGFRβ and VEGFR2, a second library was generated in which we used in vitro recombination to combine the beneficial mutations from the sequences isolated from the PDGFR and VEGFR sorts, while removing the silent and deleterious mutations. This process was employed using the staggered extension process (StEP) developed by Zhao and Arnold. Since the VEGFR sort products had a large number of mutations, we performed a second StEP reaction using only the sequences obtained from the VEGFR sorts. For the recombined PDGFR and VEGFR binding sequences (PV_{shuf}), templates from both PDGFR sort 4 and 5 were combined with templates from VEGFR sort 6 and the original PV6. For the recombined VEGFR binding sequences (V_{shuf}), templates from VEGFR sort 6 were combined with PV6. The libraries were transformed into yeast as before, creating a library of 5.1×10⁶ variants for PV_{shuf} and 1.4×10⁷ for V_{shuf}.

For the PV_{shuf} library, we performed two rounds of equilibrium sorts followed by two rounds of “off-rate” sorts (FIG. 11), alternating between PDGRβ-His and VEGFR2-Fc binding to isolate variants with increased binding affinity to both receptors. To increase the stringency of this second library sorting, monomeric PDGRβ-His was used for the PDGFR sorts. For the recombined VEGFR binding sequences (V_{shuf}), we performed two rounds of equilibrium sorts followed by two rounds of “off-rate” sorts for VEGFR2-Fc binding (FIG. 11). Post-sort analysis of the sorted cells shows that the library was enriched for clones with better binding as the sorts progressed.

Analysis of in vitro recombination library sort products. Sequencing of individual clones isolated from the PV_{shuf} library showed two consensus mutations for PDGFR binding, K87R and I182F. K87R occurred 8 times, and I182F occurred 9 times out of a total of 11 clones. Repeated mutations were also observed for VEGFR2 binding: F20L, E28G, Q71R, D160H, and D209V. From the V_{shuf} library, we can better identify the mutations for improved VEGFR2 binding. F20L and E28G occurred 12 times out of 13 clones and D160H occurred 10 times. Several other mutations occurred multiple times: M6V, Q71R, and D209V. While we identified mutations from the small sampling of sequences, more extensive sequencing would help provide a better representation of all the mutations in the population.

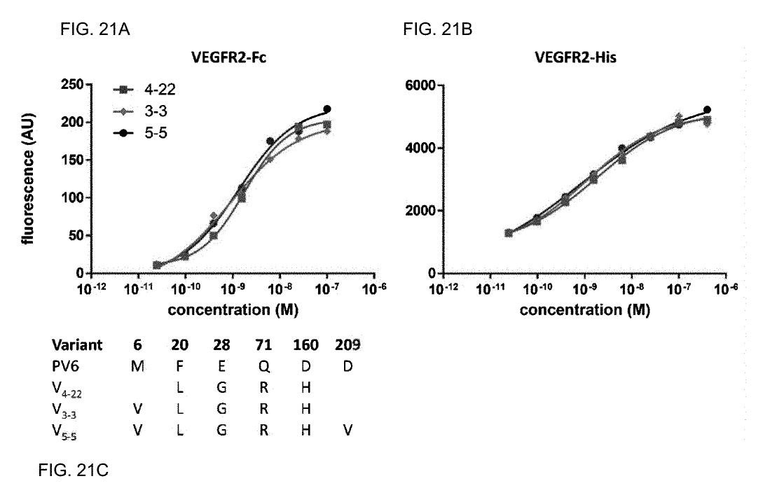

To determine whether the mutations from the VEGFR2 sorts were most beneficial for binding, we tested a few of the variants with different combinations of the M6V, Q71R, and D209V mutations (V₄₋₂₂, V₃₋₃, and V₅₋₅, FIG. 21). Both the VEGFR2-Fc used for sorting and a monomeric form of VEGFR2-His were tested. All 3 variants looked identical, indicating that M6V and D209V had less of an effect on binding. Since we want to have the minimal number of mutations, the variant V₄₋₂₂ with only 4 mutations (F2OL, E28G, Q71R, and D160H) was used for further characterization.

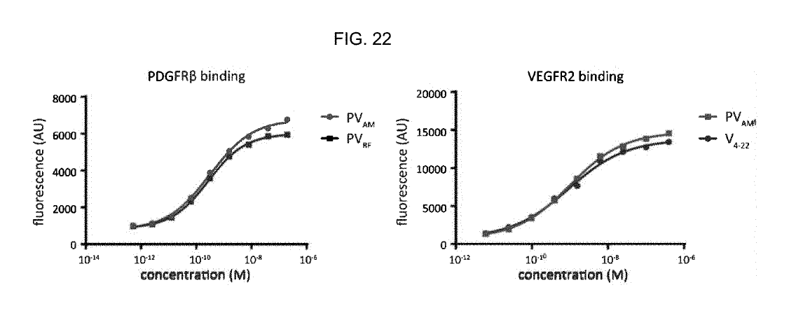

Combination of mutations for improved binding to both PDGFR and VEGFR. Since the mutations for PDGFR and VEGFR binding appeared to be additive, we combined the two mutations for improved PDGFR binding with the four mutations for VEGFR binding to create a variant that has high affinity to both PDGFR and VEGFR (named PV_{AM}). PV_{AM} had almost identical binding to PDGFR and VEGFR as the variants from which they were derived (PV_{RF} and V₄₋₂₂, respectively) (FIG. 22, Table 4). PV_{AM} and PV_{RF} bound to PDGRβ-His with K_{d} of 0.39±0.09 nM and 0.37±0.14 nM, respectively. PV_{AM} and V₄₋₂₂ bound to VEGFR2-His with K_{d} of 1.1±0.2 nM and 1.3±0.4 nM, respectively. PV6 bound to PDGRβ-His and VEGFR2-His with K_{d} of 270±70 nM and 230±50 nM, respectively. This represents an 1100-fold improvement for binding to PDGRβ-His and 200-fold improvement to VEGFR2-His.

Analysis of the mutations on the crystal structures reveals that I182F was at the PDGF-PDGFR binding interface and F20L, E28G, Q71R, and D160H were at the VEGF-VEGFR binding interface. K87R is solvent-exposed and does not engage PDGFR, but analysis of the chimera structure reveals that this mutation occurred at the interface between the PDGF and VEGF parts of the molecule. This mutation may be more beneficial for stabilizing the PDGF/VEGF chimera structure rather than direct interaction with PDGFR.

TABLE 4
Binding affinities of yeast-displayed
PDGF/VEGF variants to PDGFR and VEGFR.

| | PDGFRβ-His | VEGFR2-His |
|---|---|---|
| Variant | (nM) | (nM) |
| PV6 | 270 ± 70 | 230 ± 50 |
| PV_{RF} | 0.37 ± 0.14 | n.d. |
| V₄₋₂₂ | n.d. | 1.3 ± 0.4 |
| PV_{AM} | 0.39 ± 0.09 | 1.1 ± 0.2 |

n.d. = not determined

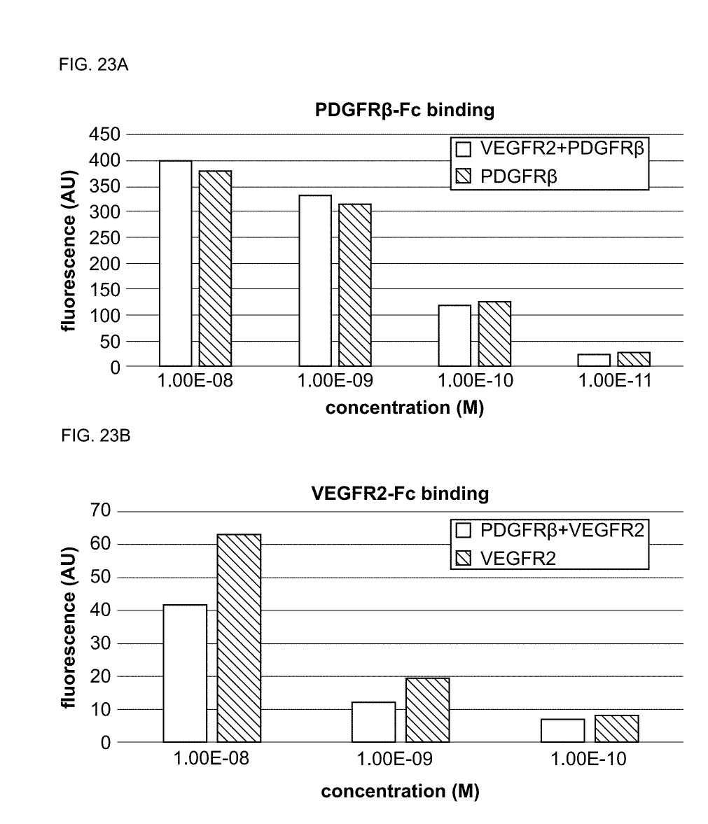

Engineered PDGFNEGF chimera binds to both PDGFR and VEGFR simultaneously. The engineered PDGFNEGF chimera was tested to see whether it can bind to both PDGFR and VEGFR simultaneously. Yeast-displayed PV_{RF} was incubated with a saturating concentration of 100 nM PDGRβ-Fc or VEGFR2-Fc and allowed to reach equilibrium. Then, various concentrations of the other receptor was added to the solution and allowed to bind. Comparison of PDGFR and VEGFR binding levels after pre-incubation with the other receptor demonstrates that the chimera is able to bind to both receptors simultaneously (FIG. 23). For PDGFR binding, there were no significant differences between binding to PV_{RF} that is already bound to VEGFR and unbound PV_{RF}. For VEGFR binding, there is a decrease in binding to PV_{RF} already bound to PDGFR compared to unbound PV_{RF}, but significant binding was observed.

We have demonstrated that it is possible to create a bispecific protein by creating a chimera of two different growth factors that are structurally similar. While both PDGF and VEGF are similar in structure, their primary sequences are divergent, with 21.6% sequence identity. PDGF and VEGF have binding interfaces composed of residues from both chains of the dimer. Hence, our design necessitated a chimeric approach of alternating patches of PDGF and VEGF to preserve their binding interfaces. Furthermore, the proteins are held together by disulfide bonds, so it was important to ensure that the chimeric structure can still form the correct disulfide bonds and fold properly.

In our initial test, only one out of five chimera designs was capable of binding to PDGFR and VEGFR, indicating that the precise crossover points must be chosen carefully as to not disrupt the native side-chain interactions. With the information gained from the first set of constructs, we designed three additional variants, all of which bound to both PDGFR and VEGFR. These results demonstrate that despite the complex design and requirements, our strategy of creating a chimeric protein composed of 11 distinct segments and 8 disulfide bonds (6 intrachain and 2 interchain disulfide bonds) was successful in creating a properly folded protein that retains the native PDGF-PDGFR and VEGF-VEGFR binding interfaces of the parent proteins. This approach is generalizable to create other multi-specific chimeras from structurally similar proteins.

We have demonstrated that our approach of random mutagenesis followed by in vitro recombination allowed for the generation of PDGF/VEGF variants with increased binding affinity to PDGFR and VEGFR. The use of yeast display allowed us to rapidly screen through a large library of 6.1×10⁷ clones by FACS. For the PDGFR sorts, two independent mutations were identified in the mutagenesis round: K87R after sort 4 and I182F after sort 5. During the in vitro recombination round, sort 4 and 5 products were recombined, and the double mutations K87R and I182F were identified to have the best binding. This result demonstrates the need to analyze intermediate sort products to ensure that we do not perform too stringent sorting and isolate only the best mutation out of the population, while losing information about other beneficial mutations. Had we analyzed just the sort 5 products, we would have only identified the I182F mutation, as K87R was eliminated after the stringent sort 5. The combination of both I182F and K87R was not observed in the first round, as that exact combination may not have existed in the original library. It would be helpful to get even more sequence information, by looking at even earlier sort products and performing more extensive sequencing to get a better representation of the whole population.

Analysis of the K87R and I182F mutations on the crystal structure of the PDGF/PDGFR complex reveals that I182F is at the interface between PDGF and PDGFR, which may help with the contact between the two interfaces. However, K87R is at the core of the molecule and not near the interface; this residue is adjacent to the cysteine at one of the PDGF/VEGF crossover points, so this mutation may help with the stability of the chimeric structure rather than the binding interaction between PDGF and PDGFR.

For the VEGFR sorts, many mutations were identified at the end of sort 6, without clear consensus mutations. The large number of repeated sequences indicated it would be not be productive to perform further sorts as our diversity had already been decreased significantly and enriched for a few mutants. Hence, sort 6 products were recombined and sorted in the V_{shuf} library. Analysis of the sort products at the end of the V_{shuf} round showed combinations of the mutations previously seen in the first library, while some mutations were eliminated. This demonstrates the effectiveness of the recombination process, where beneficial mutations are selected for while neutral or deleterious mutations are eliminated. After the V_{shuf} round, we still identified several mutations (M6V and D209V) that did not appear to improve binding. To further enrich for the best mutations, a second recombination round with wild-type sequence may help to further identify the consensus mutations. Additionally, three of the four mutations identified in our final variant PV_{AM} were identical to mutations identified in a separate screen for a VEGF variant for increased binding affinity to VEGFR, with the fourth mutation located in the same position but mutated from D to N instead of H. This further confirms that our chimera functions and binds similarly to VEGFR as the native VEGF.

Our post-sort analysis of the library products revealed that since PDGRβ-Fc has an extremely tight affinity (K_{d}˜40 pM) to PDGF, we could not distinguish the affinities between the original PV6 and the affinity-matured variants. When we used a monomeric form of the receptor PDGRβ-His, we saw weaker binding and significant affinity improvements of the variants. Hence, while a bivalent Fc fusion can be beneficial to improve the binding affinity and screening for weak binders, it is disadvantageous for screening strong binders because it limits the range that can be used to separate the highest binders from the weaker binders. In the second library round, we used PDGRβ-His for the sorts, but it may have also been beneficial to use it to screen the initial error-prone PCR library. For the characterization of the affinity matured variants to PDGFR and VEGFR described in Example 4, we use the monomeric versions of the receptors instead of the Fc fusions.

We have engineered a multi-specific protein targeting PDGFR and VEGFR by making a chimera of PDGF and VEGF, converting the ligands from agonists to antagonists. By using the natural ligands, we take advantage of the native binding interactions of the ligands to their receptors to create a single protein that inhibits both receptors. The chimera was then affinity matured to have stronger binding affinity to PDGFR and VEGFR. This resulted in engineered PDGF/VEGF variants with 1100-fold improvement in PDGFR binding and 200-fold improvement in VEGFR binding, as measured with yeast display.

### Materials and Methods

Engineering PDGF/VEGF chimera constructs. Residues 7-104 from human PDGF-B and residues 13-109 from human VEGF-A were used as the parent sequences. Using molecular visualization in PyMOL, various breakpoints were introduced at regions of structural overlap and/or conserved residues to create the PDGF/VEGF chimera. Gene constructs for these variants were synthesized using custom gene synthesis (Biomatik Corporation, Life Technologies). The genes were cloned into yeast display vector pCT with restriction sites Nhel and Mlul. Mutations for additional PDGF/VEGF variants were created by oligonucleotide site-directed mutagenesis.

Yeast surface display and binding. YPD medium contained 20 g/L dextrose, 20 g/L peptone and 10 g/L yeast extract. Selective SD-CAA medium contained 20 g/L dextrose, 6.7 g/L yeast nitrogenous base without amino acids (Difco), 5 g/L casamino acids (Bacto), 5.4 g/L Na₂HPO₄, and 8.56 g/L NaH₂PO₄·H₂O. SG-CAA induction medium is identical to SD-CAA, except with 20 g/L galactose instead of dextrose. SD-CAA plates contained the same components as the media, with the addition of 182 g/L sorbitol and 15 g/L of agar. Yeast were grown at 30° C. with shaking at 235 rpm.

Plasmids were transformed into Saccharomyces cerevisiae strain EBY100 by electroporation and recovered in YPD at 30° C. for 1 hr, then plated on SD-CAA plates. Yeast were inoculated overnight in SD-CAA and induced in SG-CAA at 30° C. for expression and display on the yeast surface, according to established protocols.

For binding experiments, 50,000 induced yeast cells were incubated with PDGRβ-Fc, PDGRβ-His, VEGFR2-His (expressed and purified as described below) or VEGFR2-Fc (R&D Systems) in phosphate-buffered saline with 1 mg/ml BSA (PBSA) at room temperature. Cells were incubated in large enough volumes to avoid ligand depletion and appropriate time to reach equilibrium. During the last hour of incubation, yeast were incubated with 1:250 dilution of chicken anti-c-Myc (Invitrogen) in PBSA. Yeast were washed and pelleted, then incubated with 1:100 dilution of secondary antibodies on ice for 10 min: anti-mouse-IgG (Fc specific)-AF488 (Invitrogen) for PDGRβ-Fc, anti-His Hilyte Fluor 488 (Anaspec) for PDGRβ-His and VEGFR-His, anti-human-IgG (Fc specific)-FITC (Sigma) for VEGFR2-Fc, and anti-chicken-IgY-PE for anti-c-Myc (Santa Cruz Biotechnology). Yeast were washed, pelleted, and resuspended in PBSA immediately before analysis by flow cytometry using BD FACSCalibur or BD Accuri C6. Flow cytometry data were analyzed using FlowJo (v7.6.1 and v10, Treestar) or BD Accuri C6 software. Statistical analysis was performed using GraphPad Prism 6.

Library creation. In the first round of library creation, a yeast surface display library was generated using error-prone PCR as described previously. PV6 was used as the template, and mutations were introduced using Taq polymerase (New England Biolabs) and nucleotide analogs 8-oxo-dGTP and dPTP (TriLink Biotech). To obtain a range of mutation frequencies, six PCRs were performed with varying concentrations of nucleotide analogs and number of PCR cycles: 5 cycles (200 μM analogs), 10 cycles (2 μM, 20 μM, 200 μM), and 20 cycles (2 μM, 20 μM). Primers consisting of 50 bp overlap with the pCT plasmid in the forward and reverse direction were designed for homologous recombination in yeast. PCR products were amplified in the absence of nucleotide analogs and purified using gel electrophoresis. pCT plasmid was digested with Nhel and Mlul. Five transformations of 5 μg purified DNA insert and 1 μg restriction enzyme digested pCT were electroporated into EBY100 competent yeast. The yeast were recovered in YPD 30° C. for 1 hr, then grown in selective SD-CAA medium. After two passages, the cells were transferred to SG-CAA to induce protein expression. A library size of 6.1×10⁷ transformants was obtained, estimated by dilution plating.

For the second library round, staggered extension process (StEP) was used to perform in vitro recombination of our error-prone PCR library sort products. For the PV_{shuf} library, templates from the PDGFR sort 4 (22.5%) and 5 (22.5%) were mixed with VEGFR sort 6 (45%) and PV6 (10%). For the V_{shuf} library, templates from VEGFR sort 6 (90%) and PV6 (10%) were mixed. PCR was performed using Taq polymerase with short cycles of 5 s extension. Thermal cycling conditions: 1 cycle at 94° C. for 30s; 60 cycles of 94° C. for 30s, 55° C. for 5s; 4° C. for ∞. PCR products were amplified and purified using gel electrophoresis. One transformation of 5 ug purified DNA and 1 ug restriction enzyme digested pCT were electroporated into EBY100 competent yeast. A library size of 5.1×10⁶ and 1.4×10⁷ transformants was obtained for PV_{shuf} and V_{shuf}, respectively, estimated by dilution plating.

Library screening. Yeast library was grown in SD-CAA media, then induced in SG-CAA at 30° C. for expression of proteins to be displayed on the yeast surface, as described above. For equilibrium binding sorts, various concentrations of soluble PDGRβ-Fc, PDGRβ-His, or VEGFR2-Fc were incubated with yeast-displayed libraries in PBSA at room temperature. Cells were incubated in large enough volumes to avoid ligand depletion and appropriate time to reach equilibrium.

For kinetic off-rate sorts, yeast were incubated with 10 nM PDGRβ-Fc, PDGRβ-His, or VEGFR2-Fc for 1 hr at room temperature, then washed with PBSA to remove unbound receptor. Cells were then resuspended in 100 nM scPDGF or scVEGFwt for PDGFR or VEGFR binding, respectively, and incubated for various times so that any receptor that dissociates from the yeast surface will be bound by the soluble competitor.

After the equilibrium binding or dissociation step of off-rate sorts, yeast were incubated with primary and secondary antibodies as described above. Labeled yeast were sorted by FACS using BD FACSAria II (Stanford Shared FACS Facility) to isolate the yeast clones with the highest binding to c-myc expression levels. Sorted clones were amplified in SD-CAA pH 4.5 media, induced for expression at SG-CAA, and subjected to further rounds of labeling and FACS to enrich the population for the highest binding clones. Sorting stringency was increased by reducing the concentration of PDGFR or VEGFR for equilibrium sorts and increasing the time for dissociation for off-rate sorts. Plasmid DNA was recovered from sorted yeast using a Zymoprep kit (Zymo Research Corp.), transformed into XL1-Blue supercompetent cells (Stratagene), and DNA was isolated using a plasmid miniprep kit (Qiagen). Sequencing was performed by Sequetech Corp. and MCLAB.

### Recombinant Protein Expression and Purification

scPDGF expression and purification. YPD medium contained 20 g/L dextrose, 20 g/L peptone and 10 g/L yeast extract. Selective SD-SCAA medium contained 20 g/L dextrose, 6.7 g/L yeast nitrogenous base without amino acids (Difco), 0.62 g/L -leu/-trp/-ura drop out supplement (Clontech), 0.04 g/L L-tryptophan (Sigma), 5.4 g/L Na₂HPO₄, and 8.56 g/L NaH₂PO₄·H₂O. SG-SCAA induction medium is identical to SD-SCAA, except with 20 g/L galactose instead of dextrose and supplemented with 0.5 g/L BSA (Roche). SD-SCAA plates contained the same components as the media, with the addition of 182 g/L sorbitol and 15 g/L of agar. Yeast were grown at 30° C. with shaking at 235 rpm.

scPDGF was expressed in Saccharomyces cerevisiae strain YVH10 that overexpresses protein disulfide isomerase to facilitate disulfide bond formation. DNA encoding residues 7-104 from human PDGF-B fused with a 14-amino acid linker was cloned into a vector containing N-terminal FLAG and C-terminal hexahistadine tags and transformed into YVH10 using electroporation. Transformed cells were recovered in 1 ml YPD at 30° C. with shaking at 235 rpm for 1 hr, then grown on selective SD-SCAA agar plates at 30° C. for 3 days. Colonies were inoculated into 5 ml SD-SCAA at 30° C. overnight and scaled up to the appropriate volumes, 500 ml in a 2 L baffled flask or 1 L in a 2.5 L baffled flask, for 36 hr until an OD₆₀₀ of 7-8. Cells were centrifuged at 4000 rpm for 10 min and resuspended in the same volume of SG-SCAA media for induction. Cells were grown for 3 days at 30° C.

Cells were centrifuged as before, and yeast supernatant was passed through a 0.22 pm membrane filter unit (Millipore). Imidazole and sodium chloride were added to the supernatant to final concentrations of 10 mM and 150 mM, respectively, and the pH was adjusted to 8.0 with sodium hydroxide. Supernatant was placed at 4° C. with stirring to allow salts in the yeast media to precipitate, then passed through a 0.22 pm membrane filter. Proteins were purified with nickel affinity chromatography using Ni-NTA agarose resin (Qiagen). 1.5 ml of resin was used per liter of supernatant. Resin was washed with 10 volumes of water, followed twice by 10 volumes of binding buffer (50 mM NaH₂PO₄, 300 mM NaCl, 10 mM imidazole, pH 8.0) and incubated with the filtered supernatant overnight at 4° C. with stirring. The mixture was then passed through a chromatography column (Kimble). Resin was washed three times with 10 volumes of binding buffer, and proteins were eluted with 10 volumes of elution buffer (50 mM NaH₂PO₄, 300 mM NaCl, 250 mM imidazole, pH 8.0). Elution fractions were concentrated with a centrifugal filter unit with 10 kDa cutoff (Amicon), and buffer exchanged into PBS to a final imidazole concentration of less than 10 mM. Proteins were further purified by size exclusion chromatography on a Varian ProStar HPLC system, using a Superdex 75 column (GE Healthcare) and PBS running buffer (Fisher) at a flow rate of 0.4 ml/min.

scVEGFwt expression and purification. scVEGFwt was expressed using the Multi-Copy Pichia Expression Kit (Invitrogen). DNA encoding residues 13-109 from human VEGF-A fused with a 14-amino acid linker was cloned into the pPIC9K vector (Invitrogen) containing N-terminal FLAG and C-terminal hexahistadine tags. Plasmid DNA was linearized by digestion with Sad. Cut plasmid and DNA insert were transformed into Pichia pastoris strain GS115 by electroporation. Transformed cells were recovered in 1 ml YPD at 30° C. with shaking at 235 rpm for 1 hr, then grown on YPD plates containing 4 mg/ml geneticin. Colonies were inoculated into 5 ml BMGY at 30° C. overnight and scaled up to the appropriate volumes, 500 ml in a 2 L baffled flask or 1 L in a 2.5 L baffled flask, until an OD₆₀₀ of 4-6. Cells were induced in BMMY at an OD₆₀₀ of 1. Cells were grown for 3 days at 30° C., adding methanol daily to maintain a concentration of approximately 0.5%. Cells were centrifuged and purified using nickel affinity chromatography and size exclusion chromatography as described above.

PDGFRβ and VEGFR2 expression and purification. PDGFRβ and VEGFR2 were expressed using the FreeStyle MAX 293 Expression System (Invitrogen). Residues 1-314 encoding the first three Ig-like domains of PDGFRβ was cloned from the cDNA (Open Biosystems, Genbank Accession ID BC032224) into the cytomegalovirus-driven pAdd2 adenoviral shuttle vector, attached to a C-terminal hexahistidine tag for PDGRβ-His or mouse IgG2a Fc domain for PDGRβ-Fc. Residues 1-764 of VEGFR2 encoding Ig-like domains 1-7 of VEGFR2 with a hexahistidine tag in mammalian expression vector pcDNA3 for VEGFR2-His. DNA was transfected into FreeStyle 293-F cells using FreeStyle MAX Reagent (Invitrogen) according to the manufacturer's protocols. Transfected cells were incubated at 37° C., 8% CO2 on an orbital shaker rotating at 135 rpm. Cells were harvested after 6 days, and the supernatants were collected for protein purification.

PDGRβ-His and VEGFR2-His were purified using nickel affinity chromatography using Ni-NTA agarose resin (Qiagen) as described above. PDGRβ-Fc was purified using Protein A chromatography. Briefly, Fc-fusion proteins were bound to Protein A-Sepharose (Invitrogen), washed with binding buffer (0.02 M NaH₂PO₄ and 0.15 M NaCl at pH 8), and eluted with pH 5 elution buffer (0.2 M NaH₂PO₄ and 0.1 M citric acid). Proteins were eluted into neutralization buffer (1 M Tris-HCl, pH 8.8) to a pH of 7.4. Proteins were buffer exchanged into PBS using an Amicon Ultra-4 Centrifugal Filter Unit with 10 kDa membrane (Millipore). Proteins were further purified by size exclusion chromatography on a Varian ProStar HPLC system, using a Superdex 200 Increase column (GE Healthcare) and PBS running buffer (Fisher) at a flow rate of 0.4 ml/min. The final concentration of protein was determined by absorbance at 280 nm.

### Example 4

### Characterization of Engineered PDGF/EGF Chimera

Recombinant expression of PDGFNEGF variants. To characterize the engineered PDGFNEGF variants in the soluble form off of the yeast surface, PV6 and _{PVAM} were recombinantly expressed in Saccharomyces cerevisiae strain YVH10 that overexpresses protein disulfide isomerase to facilitate disulfide bond formation. The engineered PDGFNEGF constructs were cloned from the yeast display vector into a yeast secretion vector with N-terminal FLAG and C-terminal hexahistidine tags to facilitate purification of the proteins and detection in binding assays. Yeast was transformed with the DNA, grown in selective SD-SCAA media, then induced in galactose containing SG-SCAA. Secreted protein from the yeast supernatant were purified using nickel affinity chromatography. The protein was then treated with Endo H_{f} to cleave yeast glycosylation, followed by size exclusion chromatography. The overall yield was approximately 1 mg/L of yeast culture. Several bands were observed when the purified protein was run on a reducing SDS-PAGE but not in non-reducing conditions, indicating cleavage of the polypeptide that remains held together by disulfide bonds (FIG. 13A).

PDGF/VEGF variants with increased stability and high expression. PDGF-B is known to have a protease site at arginine-32 (corresponding to arginine-137 in PV6). This residue was mutated to alanine to remove the protease site. An alternate site was also identified at arginine-28 (corresponding to arginine-133); this residue was mutated to serine. Comparison of R137A, R133S, and the double mutant showed that R137 was the cause of the multiple bands (FIG. 13B). We termed the R137A variant PV_{AM-S}. Additionally, we found that degradation was significantly eliminated upon expression of this protein at 20° C. As further evidence that the original cleavage site was at R137, the reduced PV6 sample was analyzed by matrix-assisted laser desorption/ionization time-of-flight (MALDI-TOF) mass spectrometry. A peak with mass of 9176 Da was observed, consistent with the expected masses of 16721 and 9177 Da if the protein were cleaved at R137 in PV6.

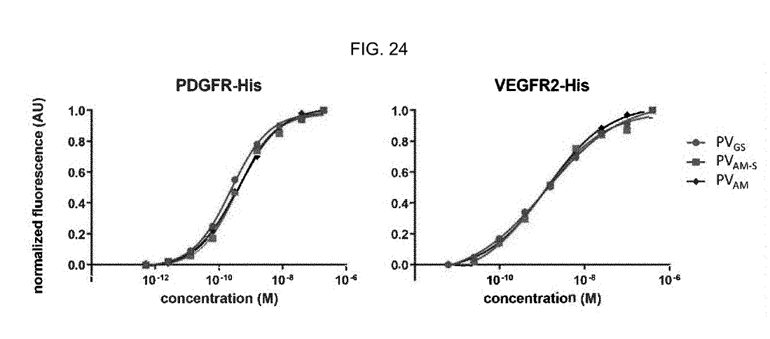

While the R137A mutation and expression at 20° C. removed the most prominent bands on the reducing SDS-PAGE, there was an additional band that remained, seen most prominently in the protein expressed at 30° C. (FIG. 13B). Edman sequencing of the band revealed that the degradation site was in the 14-aa linker joining the two chains, with the sequence SSEGKGE identified. Hence, we replaced the linker with an inert (Gly₄Ser)₄ linker to create PV_{GS}. We noticed that the length of the original 14-aa linker barely spanned the distance between the two chains of the dimer; hence, the linker was lengthened to 20 amino acids to help provide more flexibility for the protein to form the correct dimerization of the single-chain construct. Expression of this variant showed a clean product (FIG. 13C, D), and the expression yield was significantly improved compared to PV_{AM-S}. Expression of PV_{AM-S} at 20° C. had decreased the yield to 200 pg/L of culture, and PV_{GS} increased the yield to 1.8 mg/L at 20° C. Yeast display binding showed that all the various changes to the protein had negligible effects on the binding affinity to PDGFR and VEGFR (FIG. 24, Table 5).

TABLE 5
Binding affinities of yeast-displayed
PDGF/VEGF variants to PDGFR and VEGFR.

| | PDGFRβ-His | VEGFR2-His |
|---|---|---|
| Variant | (nM) | (nM) |
| PV_{AM} | 0.39 ± 0.09 | 1.1 ± 0.2 |
| PV_{AM-S} | 0.34 ± 0.05 | 1.0 ± 0.4 |
| PV_{GS} | 0.25 ± 0.04 | 1.1 ± 0.4 |

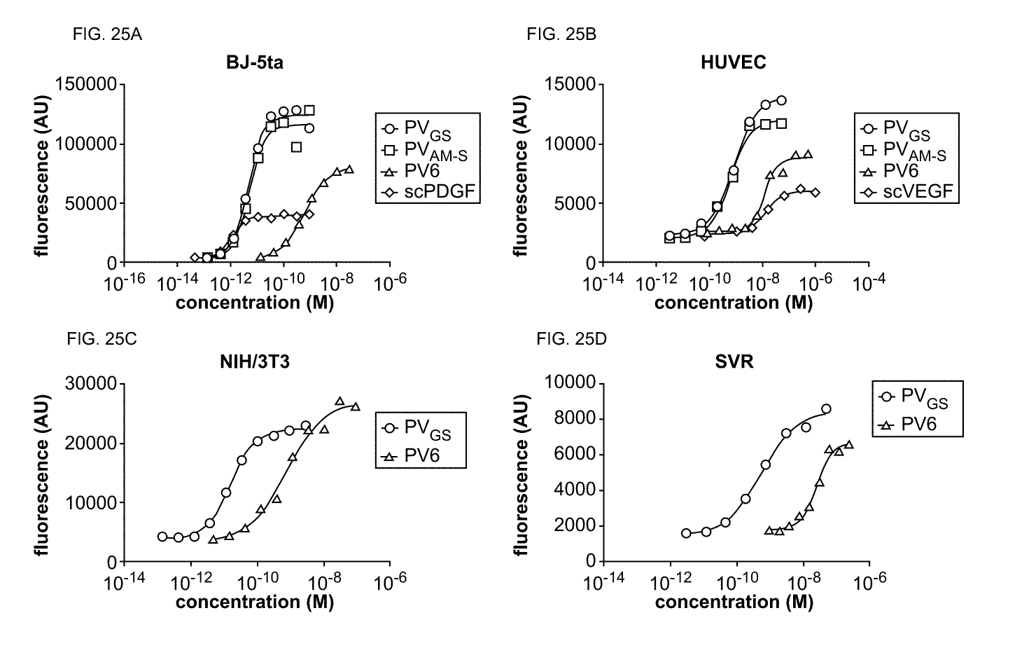

Binding of engineered PDGFNEGF chimera to PDGFR and VEGFR expressing cells. The recombinantly expressed PDGF/VEGF chimeras were measured for their binding affinity to PDGFR and VEGFR expressed on the surface of several mammalian cell lines. To test binding to PDGFR, human and mouse fibroblasts BJ-5ta and NIH/3T3, respectively, were used. Various concentrations of PDGFNEGF chimera were incubated with the cells in binding buffer for 5 hr at 4° C. Cells were then washed and stained with fluorescently-labeled anti-FLAG antibody, and cells were analyzed by flow cytometry. For BJ-5ta, a K_{d} of 5.0±0.7 pM was observed for PV_{GS}, a 70-fold improvement in affinity compared to a K_{d} of 350±40 pM for PV6 (FIG. 25A, Table 6). PV_{AM-S} had an almost identical K_{d} of 5.1±0.6 pM. This is comparable but weaker binding compared to the bivalent single-chain PDGF-BB (scPDGF), which has a K_{d} of 1.5±0.3 pM. For NIH/3T3, a K_{d} of 19±6 pM was observed, which is a 53-fold improvement compared to 1.0±0.4 nM PV6 (FIG. 25C, Table 6). This indicates that the mutations that conferred affinity improvement to human PDGFR also improved affinity to mouse PDGFR.

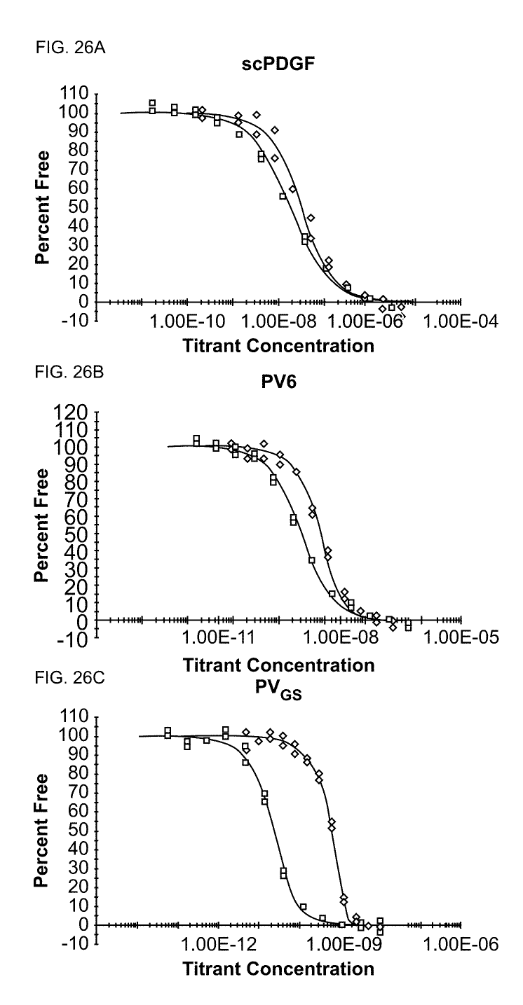

To test binding to VEGFR, human umbilical vein endothelial cells (HUVECs) and murine angiosarcoma endothelial cells (SVR) were used. For HUVECs, cells could only be incubated for 3 hr due to high cell death at longer incubations. A K_{d} of 0.77±0.04 nM was observed for PV_{GS}, compared to 11±3 nM for PV6, a 14-fold improvement in affinity (FIG. 26B, Table 6). PV_{AM-S} had a K_{d} of 0.53±0.08 nM, and single-chain VEGF-A (scVEGF) had a K_{d} of 18±9 nM. For SVR, a K_{d} of 0.45±0.10 nM was observed for PV_{GS}, a 78-fold improvement in affinity compared to a K_{d} of 35±22 nM for PV6 (FIG. 25D, Table 6).

While binding to cell expressed surface receptors provides a relevant biological system to assess the binding affinity, we were limited by the short incubation times (3-5 hours) for the binding reactions due to cell death during the binding incubation. The binding reactions do not reach equilibrium and thus true binding affinity is not measured. In addition, for the tight picomolar binding to PDGFR cells, there may be problems with ligand depletion. While we tried to avoid ligand depletion by incubating with increasing volumes of ligand at lower concentrations, we were limited from using larger volumes beyond the 50 ml incubations that were used with the lowest concentrations.

TABLE 6
Binding affinities of PDGF/VEGF variants
to PDGFR and VEGFR expressing cells.

| | HUVEC (nM) | SVR (nM) | BJ-5ta (pM) | NIH/3T3 (pM) |
|---|---|---|---|---|
| | human | mouse | human | mouse |
| Variant | VEGFR | VEGFR | PDGFR | PDGFR |
| scVEGF | 18 ± 9 | 10 ± 5 | n.d | n.d. |
| scPDGF | n.d. | n.d. | 1.5 ± 0.3 | 2.7 ± 1.8 |
| PV6 | 11 ± 3 | 35 ± 22 | 350 ± 40 | 1000 ± 400 |
| PV_{AM-S} | 0.53 ± 0.08 | n.d. | 5.1 ± 0.6 | n.d. |
| PV_{GS} | 0.77 ± 0.04 | 0.45 ± 0.10 | 5.0 ± 0.7 | 19 ± 6 |

n.d. = not determined

Binding affinity and kinetics of PDGF/VEGF chimera to PDGFR and VEGFR using kinetic exclusion assay. Given the limitations of measuring accurate binding affinity using a yeast-displayed construct that is tethered to the yeast or measuring binding to mammalian cells, we measured the binding affinity of the engineered PDGF/VEGF chimeras using the kinetic exclusion assay (KinExA). KinExA can be used to measure the equilibrium binding affinity (K_{d}) and the association rate constant (kₒₙ), from which the dissociation rate constant (k_{off}) can be calculated. For KinExA measurements, a constant amount of PDGF/VEGF ligand is titrated with differing concentrations of PDGRβ-His or VEGFR2-His. The solutions are incubated for an appropriate amount of time to allow the ligand-receptor binding reaction to reach equilibrium. The solution is then briefly exposed to a solid support containing immobilized PDGFR or VEGFR, which captures unbound ligand. The amount of ligand bound to the solid support is then detected using a fluorescently-labeled antibody. The short contact time does not allow for dissociation of pre-formed ligand-receptor complexes in solution; hence, competition between solution PDGFR/VEGFR and solid phase receptor is “kinetically excluded,” and the detected signal represents only the amount of unbound ligand in solution. For each binding interaction measured, curves for two concentrations of the constant ligand are performed. By performing two experiments with fixed ligand concentrations above and below the K_{d}, and fitting both sets of data with a nonlinear regression analysis, a more accurate measurement for the affinity can be obtained.

Additionally, the kinetic parameter kₒₙ can be determined by using a “kinetics injection” experiment. A constant concentration of PDGF/VEGF ligand is injected with an equal volume of differing concentrations of PDGRβ-His or VEGFR2-His. The two solutions are allowed to mix briefly (several seconds) in the time required for the instrument to deliver the mixture to the detection bead column. The pre-equilibrium concentrations of free ligand is fit to the standard bimolecular rate equation to obtain kₒₙ.

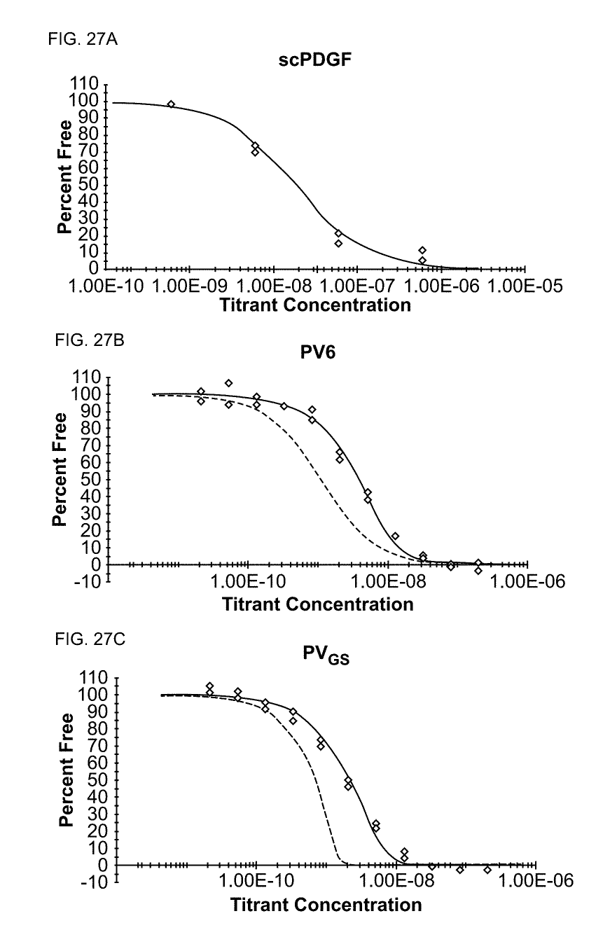

For PDGRβ-His binding, a K_{d} of 18 nM was observed for scPDGF. For PV6, a K_{d} of 870 pM was observed, while PV_{GS} had a K_{d} of 6.7 pM, representing a 130-fold improvement in binding affinity (FIG. 26, Table 7). The on-rates were extremely fast, 2.2×10⁷ M⁻¹s⁻¹ for PV6 and 2.0×10⁷ M⁻¹s⁻¹ for PV_{GS} (FIG. 27, Table 7). This indicates that the improvement in affinity is entirely attributed to a decrease in off-rate. Interestingly, the scPDGF binding reaction reached equilibrium in under 5 s (FIG. 27A), which is expected if scPDGF has the same on-rate as PV6 and PV_{GS}. The fast equilibrium time precluded the use of pre-equilibrium kinetic analysis by KinExA for scPDGF.

TABLE 7

| Variant | K_{d} (M) | kₒₙ (10⁷ M⁻¹ s⁻¹) | k_{off} (10⁻⁴ s⁻¹) |
|---|---|---|---|
| scPDGF | 18 (12-27) × 10⁻⁹ | >1.7* | >3100* |
| PV6 | 870 (530-1350) × 10⁻¹² | 2.2 (1.8-2.7) | 190 (160-240) |
| PV_{GS} | 6.7 (3.3-11.7) × 10⁻¹² | 2.0 (1.6-2.5) | 1.3 (1.1-1.7) |

Numbers in parentheses represent the 95% confidence interval.
*Could not be accurately determined with a pre-equilibrium “kinetics injection” experiment due to fast equilibrium time.

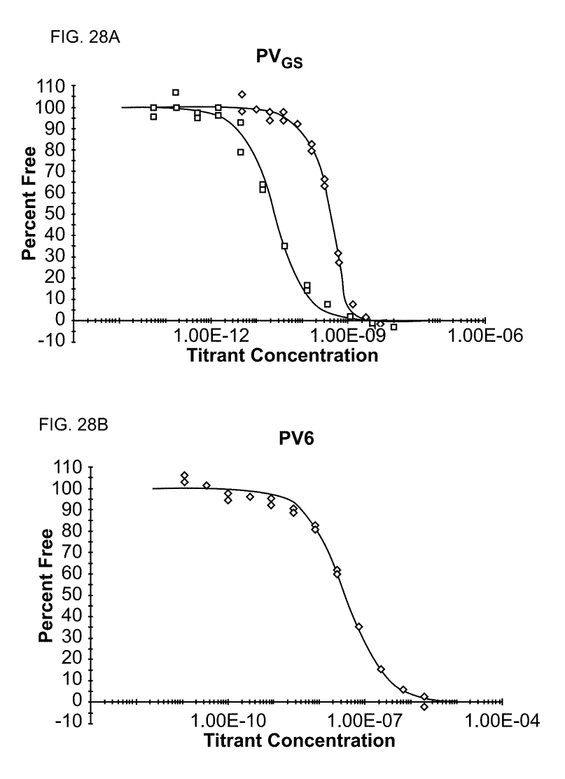

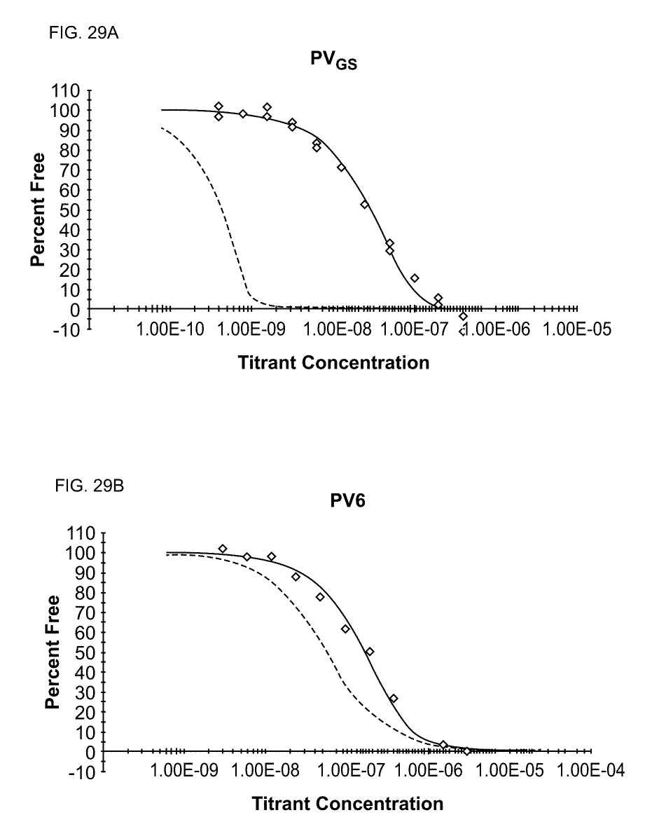

For VEGFR2-His binding, a K_{d} of 38 nM was observed for PV6, while PV_{GS} had a K_{d} of 14 pM. This represents a 2700-fold improvement in affinity, a significant increase compared to what was observed yeast-display and mammalian cell binding (FIG. 28, Table 8. The on-rates were 2.4×10⁵ M⁻¹s⁻¹ for PV6 and 6.8×10⁵ M⁻¹s⁻¹ for PV_{GS} (FIG. 29, Table 8. As seen with PDGFR binding, the improvement in affinity is mostly attributed to a decrease in off-rate.

TABLE 8
Binding affinities and rate constants of PDGF/VEGF
variants to VEGFR2, measured by KinExA.

| Variant | K_{d} (M) | kₒₙ (10⁵ M⁻¹ s⁻¹) | k_{off} (10 s⁻¹) |
|---|---|---|---|
| PV6 | 38 (27-47) × 10⁻⁹ | 2.4 (2.0-3.0) | 9.2 (7.6-11.4) × 10⁻³ |
| PV_{GS} | 14 (9-22) × 10⁻¹² | 6.8 (5.6-8.1) | 9.6 (8.0-11.4) × 10⁻⁶ |

Numbers in parentheses represent the 95% confidence interval.

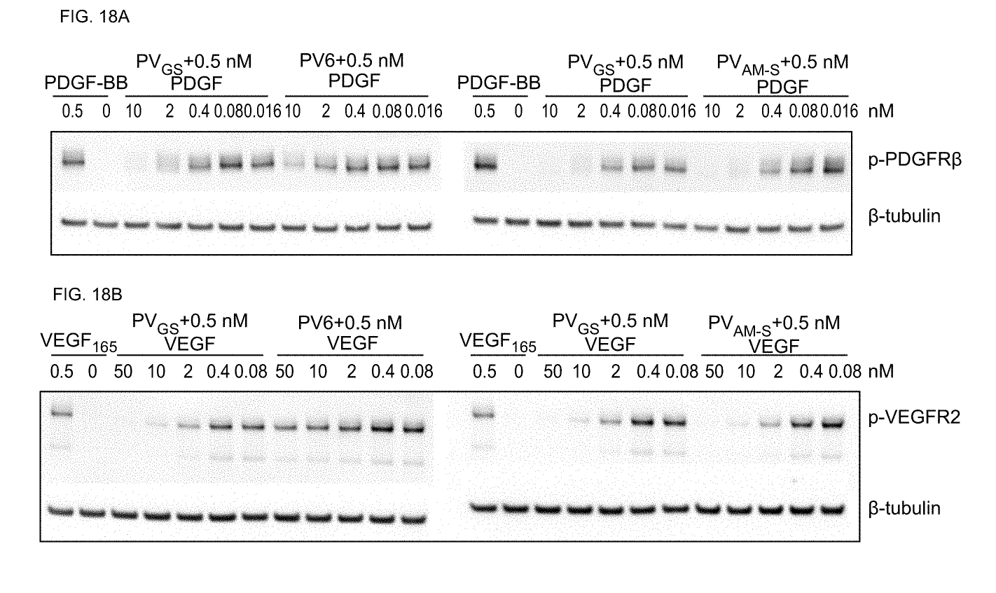

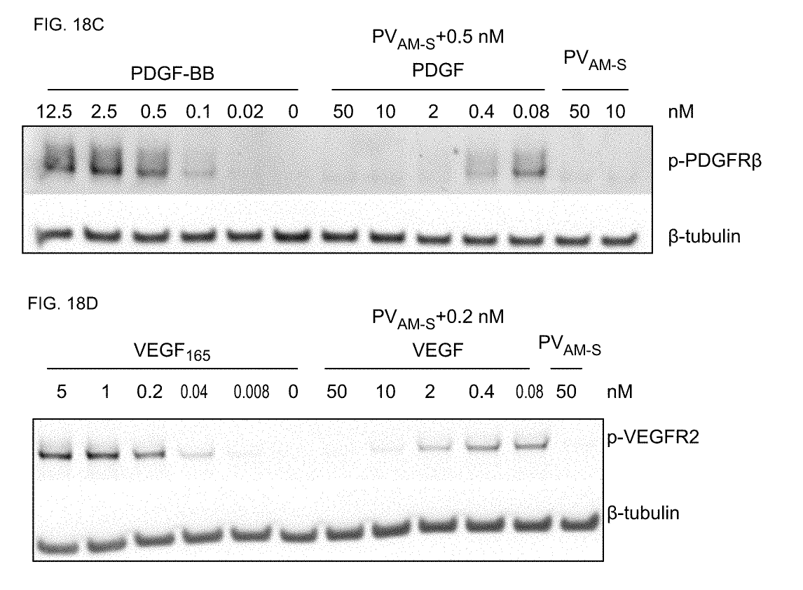

PDGFNEGF chimera inhibits PDGF- and VEGF-mediated phosphorylation of PDGFR/3 and VEGFR2. After verifying that the PDGFNEGF chimeras bind to cell surface expressed receptors, the chimeras were tested for their ability to inhibit PDGF- and VEGF-induced phosphorylation of PDGFR and VEGFR2, respectively. Serum-starved BJ-5ta cells were stimulated with 0.5 nM PDGF-BB in the presence of various concentrations of PV_{GS}, PV_{AM-S}, and PV6 for 10 min. Cells were lysed, and western blot was used to detect levels of phosphorylated PDGFRβ in the cell lysates with an antibody that detects phosphorylated PDGFRβ at Tyr1021. PV_{GS} and PV_{AM-S} show complete inhibition of PDGFRβ phosphorylation to baseline levels at a concentration of 10 nM, and PV6 inhibits to a lesser degree (FIG. 18A, C). Serum-starved HUVECs were stimulated with 0.5 nM VEGF₁₆₅ in the presence of PV_{GS}, PV_{AM-S}, and PV6 for 8 min, and phosphorylated VEGFR2 was detected with an antibody against phosphorylated VEGFR2 at Tyr1175. PV_{GS} and PV_{AM-S} show complete inhibition of VEGFR2 phosphorylation to baseline levels at a concentration of 50 nM, while PV6 only weakly inhibits (FIG. 18B, D).

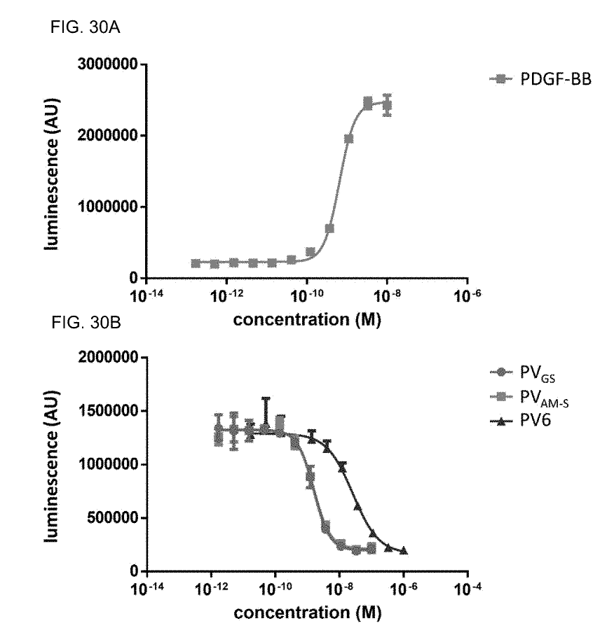

PDGFNEGF chimera inhibits PDGF-mediated phosphorylation in reporter cell line. In addition to western blot analysis, stimulation of PDGFRβ phosphorylation was detected using the DiscoveRx PathHunter reporter cell line. DiscoveRx PDGFRβ cells were stimulated with 0.5 nM PDGF-BB in the presence of various concentrations of PV_{GS}, PV_{AM-S}, and PV6 for 3 hr, followed by cell lysis and detection with PathHunter reagent and luminescence readout. PDGF-BB stimulated DiscoveRx PDGFRβ cells with an EC₅₀ of 0.54±0.16 nM. PV_{GS}, PV_{AM-S}, and PV6 completely inhibited PDGF-BB stimulation of PDGFR, with an IC₅₀ of 1.1±0.6 nM, 1.4±0.3 nM, and 23±10 nM, respectively (FIG. 30).

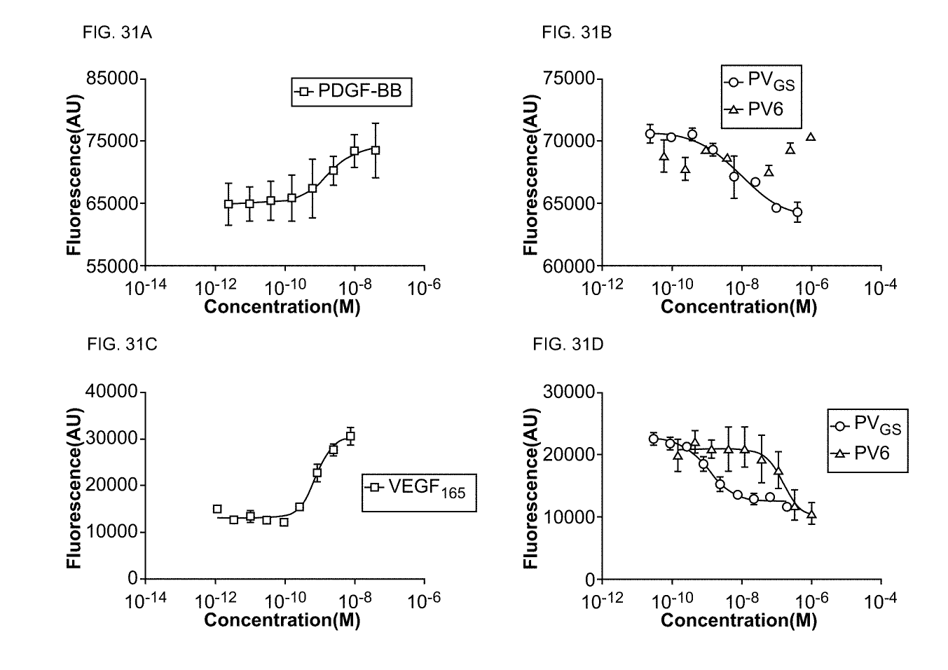

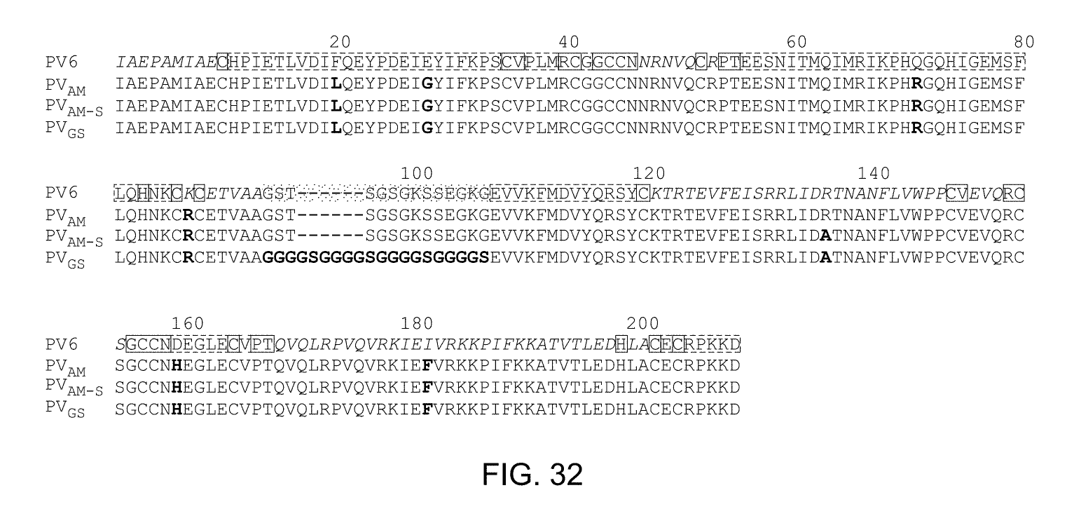

PDGFNEGF chimera inhibits PDGF- and VEGF-mediated cell proliferation. PDGFNEGF chimeras were tested for their ability to inhibit PDGF- and VEGF-induced cell proliferation. After serum-starvation, BJ-5ta cells were stimulated with 5 nM PDGF-BB in the presence of PV_{GS} and PV6. Addition of 10 pg/ml insulin to the assay media improved the dynamic range of the assay. After 48 hr, alamarBlue was added to measure cell proliferation, as described in Chapter 3. PDGF-BB stimulated BJ-5ta cell proliferation with an EC₅₀ of 2.7±0.9 nM (FIG. 31A). PV_{GS} showed complete inhibition of PDGF-induced cell proliferation with an IC50 of 14±10 nM, while PV6 did not show any inhibition (FIG. 31B).

For HUVECs, serum-starved cells were stimulated with 0.5 nM VEGF₁₆₅ in the presence of PV_{GS} and PV6. Addition of 1% FBS to the serum starvation media and 10 μg/ml insulin to the assay media improved the dynamic range of the assay. VEGF₁₆₅ stimulated HUVEC proliferation with an EC₅₀ of 0.67±0.15 nM (FIG. 31C). Both PV_{GS} and PV6 showed complete inhibition of VEGF-induced cell proliferation with an IC₅₀ of 1.2±0.3 nM and 110±60 nM, respectively (FIG. 31D).

All of the results collectively support the conclusion that our engineering strategy was successful. The receptor phosphorylation and cell proliferation assays demonstrate that the engineered PDGF/VEGF chimera functions as an antagonist of PDGFR and VEGFR. Furthermore, the affinity-matured variant PV_{GS} is a more effective antagonist compared to PV6. These promising in vitro results provide the rationale for use in vivo. While these proteins were engineered to bind with high affinity to human receptors, they also showed improved binding affinity to mouse PDGFR and VEGFR, allowing these molecules to be tested in animal models.

We were successful in our approach to rationally engineer a chimera of two growth factors for creating a multi-specific protein. Using directed evolution, the proteins were engineered to have increased affinity to PDGFR and VEGFR. KinExA measurements revealed that these proteins have significantly stronger affinity than the original chimera. The engineered chimera was a potent anatagonist of PDGF and VEGF stimulation in cell models, demonstrating utility for anti-angiogenesis therapy.

### Materials and Methods

Recombinant protein expression and purification. scPDGF and PDGF/VEGF chimera expression and purification. YPD medium contained 20 g/L dextrose, 20 g/L peptone and 10 g/L yeast extract. Selective SD-SCAA medium contained 20 g/L dextrose, 6.7 g/L yeast nitrogenous base without amino acids (Difco), 0.62 g/L -leu/-trp/-ura drop out supplement (Clontech), 0.04 g/L L-tryptophan (Sigma), 5.4 g/L Na₂HPO₄, and 8.56 g/L NaH₂PO₄·H₂O. SG-SCAA induction medium is identical to SD-SCAA, except with 20 g/L galactose instead of dextrose and supplemented with 0.5 g/L BSA (Roche). SD-SCAA plates contained the same components as the media, with the addition of 182 g/L sorbitol and 15 g/L of agar. Yeast were grown at 30° C. with shaking at 235 rpm.

scPDGF and PDGF/VEGF were expressed in Saccharomyces cerevisiae strain YVH10 that overexpresses protein disulfide isomerase to facilitate disulfide bond formation. DNA constructs were cloned into a vector containing N-terminal FLAG and C-terminal hexahistadine tags and transformed into YVH10 using electroporation. Transformed cells were recovered in 1 ml YPD at 30° C. with shaking at 235 rpm for 1 hr, then grown on selective SD-SCAA agar plates at 30° C. for 3 days. Colonies were inoculated into 5 ml SD-SCAA at 30° C. for 24 hr and scaled up to the appropriate volumes, 500 ml in a 2 L baffled flask or 1 L in a 2.5 L baffled flask, for 36 hr until an 0D₆₀₀ of 7-8. Cells were centrifuged at 4000 rpm for 10 min and resuspended in the same volume of SG-SCAA media for induction. Cells were grown for 3 days at 20° C. for PV_{GS} or 30° C. for the other constructs.

Cells were centrifuged as before, and yeast supernatant was passed through a 0.22 μm membrane filter unit (Millipore). Imidazole and sodium chloride were added to the supernatant to final concentrations of 10 mM and 150 mM, respectively, and the pH was adjusted to 8.0 with sodium hydroxide. Supernatant was placed at 4° C. with stirring to allow salts in the yeast media to precipitate, then passed through a 0.22 pm membrane filter. Proteins were purified with nickel affinity chromatography using Ni-NTA agarose resin (Qiagen). 1.5 ml of resin was used per liter of supernatant. Resin was washed with 10 volumes of water, followed twice by 10 volumes of binding buffer (50 mM NaH₂PO₄, 300 mM NaCl, 10 mM imidazole, pH 8.0) and incubated with the filtered supernatant overnight at 4° C. with stirring. The mixture was then passed through a chromatography column (Kimble). Resin was washed three times with 10 volumes of binding buffer, and proteins were eluted with 10 volumes of elution buffer (50 mM NaH₂PO₄, 300 mM NaCl, 250 mM imidazole, pH 8.0). Elution fractions were concentrated with a centrifugal filter unit with 10 kDa cutoff (Amicon), and buffer exchanged into PBS to a final imidazole concentration of less than 10 mM. For PDGF/VEGF chimera, N-linked glycosylation was removed by digestion with Endo H_{f} (New England Biolabs) for 2 hours at 37° C. Proteins were further purified by size exclusion chromatography on a Varian ProStar HPLC system, using a Superdex 75 column (GE Healthcare) and PBS running buffer at a flow rate of 0.4 ml/min.

scVEGFwt expression and purification. scVEGFwt was expressed using the Multi-Copy Pichia Expression Kit (Invitrogen). DNA encoding residues 13-109 from human VEGF-A fused with a 14-amino acid linker was cloned into the pPIC9K vector (Invitrogen) containing N-terminal FLAG and C-terminal hexahistadine tags. Plasmid DNA was linearized by digestion with Sad. Cut plasmid and DNA insert were transformed into Pichia pastoris strain GS115 by electroporation. Transformed cells were recovered in 1 ml YPD at 30° C. with shaking at 235 rpm for 1 hr, then grown on YPD plates containing 4 mg/ml geneticin. Colonies were inoculated into 5 ml BMGY at 30° C. overnight and scaled up to the appropriate volumes, 500 ml in a 2 L baffled flask or 1 L in a 2.5 L baffled flask, until an OD₆₀₀ of 4-6. Cells were induced in BMMY at an OD₆₀₀ of 1. Cells were grown for 3 days at 30° C., adding methanol daily to maintain a concentration of approximately 0.5%. Cells were centrifuged and purified using nickel affinity chromatography and size exclusion chromatography as described above.

PDGFRβ and VEGFR2 expression and purification. PDGFRβ and VEGFR2 were expressed using the FreeStyle MAX 293 Expression System (Invitrogen). Residues 1-314 encoding the first three Ig-like domains of PDGFRp was cloned from the cDNA (Open Biosystems, Genbank Accession ID BC032224) into the cytomegalovirus-driven pAdd2 adenoviral shuttle vector, attached to a C-terminal hexahistidine tag for PDGFRβ-His or mouse IgG2a Fc domain for PDGFRβ-Fc. Residues 1-764 of VEGFR2 encoding Ig-like domains 1-7 of VEGFR2 with a hexahistidine tag in mammalian expression vector pcDNA3 for VEGFR2-His. DNA was transfected into FreeStyle 293-F cells using FreeStyle MAX Reagent (Invitrogen) according to the manufacturer's protocols. Transfected cells were incubated at 37° C., 8% CO2 on an orbital shaker rotating at 135 rpm. Cells were harvested after 6 days, and the supernatants were collected for protein purification.

PDGFRβ-His and VEGFR2-His were purified using nickel affinity chromatography using Ni-NTA agarose resin (Qiagen) as described above. PDGFRβ-Fc was purified using Protein A chromatography. Briefly, Fc-fusion proteins were bound to Protein A-Sepharose (Invitrogen), washed with binding buffer (0.02 M NaH₂PO₄ and 0.15 M NaCl at pH 8), and eluted with pH 5 elution buffer (0.2 M NaH₂PO₄ and 0.1 M citric acid). Proteins were eluted into neutralization buffer (1 M Tris-HCl, pH 8.8) to a pH of 7.4. Proteins were buffer exchanged into PBS using an Amicon Ultra-4 Centrifugal Filter Unit with 10 kDa membrane (Millipore). Proteins were further purified by size exclusion chromatography on a Varian ProStar HPLC system, using a Superdex 200 Increase column (GE Healthcare) and PBS running buffer (Fisher) at a flow rate of 0.4 ml/min. The final concentration of protein was determined by absorbance at 280 nm.

Kinetic Exclusion Assay (KinExA). All experiments were performed on a KinExA 3200 instrument with siliconized flow cell (Sapidyne Instruments Inc). Recombinantly expressed PDGFRβ-Fc or VEGFR2-Fc were conjugated to activated azlactone beads (Sapidyne Instruments Inc). 30 μg of receptor was incubated in 1 ml of 50 mM sodium bicarbonate, 0.5 M sodium citrate buffer, pH 8.3, for 2 hr at room temperature with rocking. Beads were allowed to settle by gravity and liquid removed. Beads were blocked by incubating in 1 ml of 10 mg/ml BSA in 1 M Tris buffer, pH 8.0, for 1 hr at room temperature with rocking, and stored in blocking buffer at 4° C. for up to one week until use. Running buffer contained PBS with 10 mM imidazole and 0.02% sodium azide. Samples were prepared in running buffer with 1 mg/ml BSA. Binding reactions were prepared with a fixed concentration of PDGF/VEGF ligands and dilution series of PDGFR or VEGFR. Captured PDGF/VEGF ligands were detected using a Dylight 649-conjugated anti-FLAG antibody (Rockland) at 1:1000 dilution in sample buffer. Kinetic experiments were performed with the “Kinetics Injection” method defined in the KinExA software. Data were analyzed with KinExA Pro Software. The K_{d} was determined from a global fit of two concentration curves using the “n-curve analysis” tool and the titrant concentrations as reference. The software reports a best fit value and calculates the 95% confidence intervals for the K_{d} and active binding site concentration. The kₒₙ was calculated as a fit of the pre-equilibrium free ligand concentrations to the bimolecular binding equation using the “Kinetics Injection” tool.

Cell culture conditions. BJ-5ta cells (ATCC CRL-4001) were grown in 4 parts Dulbecco's Modified Eagle Medium (DMEM, Gibco 11995) and 1 part M199 (Gibco 11150), supplemented with 10% fetal bovine serum (FBS, Gibco 10437) and 0.01 mg/ml hygromycin B (Gibco 10687). HUVECs (Lonza CC-2519) were grown in EGM BulletKit Media (Lonza CC-3124). NIH/3T3 cells (ATCC CRL-1658) were grown in DMEM (Gibco 11995) with 10% calf serum (Gibco 16010). SVR cells (ATCC CRL-2280) were grown in DMEM (ATCC 30-2002), supplemented with 5% FBS and 1% Pen-Strep (Gibco 15140).

Cell-binding assay. Varying concentrations of scPDGF, scVEGF, and PDGF/VEGF ligands were incubated with 25,000 PDGFR- or VEGFR-expressing cells in binding buffer (20 mM Tris pH 7.5 with 1 mM MgCl₂, 1 mM MnCl₂, 2 mM CaCl₂, 100 mM NaCl, and 1 mg/ml BSA) for 5 hr (BJ-5ta, NIH/3T3) or 3 hr (HUVEC, SVR) at 4° C. Cells were washed and pelleted, then incubated with 1:100 R-PE-conjugated anti-FLAG antibody (Prozyme) for 15 min on ice. Cells were washed, pelleted, and resuspended in binding buffer immediately before analysis by flow cytometry using BD Accuri C6. Flow cytometry data were analyzed using BD Accuri C6 software, and statistical analysis was performed using GraphPad Prism 6.

PDGFR phosphorylation assay. BJ-5ta cells were seeded on 6-well plates at a density of 50,000 cells/well. After 24 hr, cells were replaced with serum-free media (SFM) containing 4 parts DMEM, 1 part M199, and 0.1% BSA for 24 hr. Cells were stimulated with PDGF-BB (Peprotech) and PDGF/VEGF chimera in SFM for 10 min at 37° C. in the presence of phosphatase inhibitor cocktail 2 (Sigma). Cells were washed with ice-cold PBS and treated with 100 μl of lysis buffer (20 mM Tris-HCl, pH 8.0, 137 mM NaCl, 10% Glycerol, 1% Nonidet P-40) with 1× phosphatase inhibitor cocktail 2 and 1× protease inhibitor cocktail 2 (Sigma) for 1 hr at 4° C. Lysates were frozen at −80° C., clarified by centrifugation, and quantified with BCA assay (Pierce) to normalize for protein concentration.

Cell lysates were separated by SDS-PAGE, followed by transfer to nitrocellulose membrane using the iBlot module (Invitrogen). Immunoblots were incubated in 5% milk (Bio-Rad) in Tris-buffered saline (20 mM Tris-HCl, 150 mM NaCl, pH 7.6) with 1 mg/L Tween 20 (TBST), probed with primary antibodies overnight at 4° C., washed with TBST, then probed with secondary antibodies for 1 hr at room temperature. Primary antibodies used were anti-phospho-PDGFRβ (Cell Signaling 2227) at 1:1000 dilution and anti-β-tubulin (Covance MMS-410P) at 1:10,000 dilution. Secondary antibodies conjugated with horseradish peroxidase used were anti-rabbit (Jackson Immunoresearch 711-035-152) at 1:5000 dilution for anti-phospho-PDGFRβ and anti-mouse (Jackson Immunoresearch 715-035-150) at 1:50,000 for anti-β-tubulin. Immunoblots were developed using SuperSignal West Femto (Thermo), and chemiluminescence was detected on a ChemiDoc XRS+ System (Bio-Rad).

VEGFR phosphorylation assay. HUVECs were seeded on 6-well plates at a density of 100,000 cells/well in EGM BulletKit Media. After 24 hr, cells were replaced with serum-starvation media (SFM): EBM (Lonza CC-3121) with 0.2% BSA and 0.5% FBS for 12 hr. Cells were stimulated with VEGF₁₆₅ (Peprotech) and PDGF/VEGF chimera in SFM for 8 min at 37° C. in the presence of phosphatase inhibitor cocktail 2 (Sigma). Cells were washed with ice-cold HEPES-BSS (Lonza CC-5024), and treated with lysis buffer with 1× phosphatase inhibitor cocktail 2 and 1× protease inhibitor cocktail 2 (Sigma) for 1 hr at 4° C. Immunoblotting of cell lysates was performed as described above. Primary antibodies used were anti-phospho-VEGFR2 Y1175 (Cell Signaling 2478) at 1:1000 dilution and anti-β-tubulin (Covance MMS-410P) at 1:10,000 dilution. Secondary antibodies conjugated with horseradish peroxidase used were anti-rabbit (Jackson Immunoresearch 711-035-152) at 1:5000 dilution for anti-phospho-VEGFR2 and anti-mouse (Jackson Immunoresearch 715-035-150) at 1:50,000 for anti-β-tubulin.

DiscoveRx PathHunter cell-based kinase assay. PathHunter U2OS PDGFRβ cells (DiscoveRx) were cultured in PathHunter select U2OS cell culture media K (DiscoveRx). The PathHunter cell-based assay was performed according to DiscoveRx protocols. Cells were plated in PathHunter Cell Plating 16 media on 384-well plates at a density of 10,000 cells/well. After 24 hr, cells were stimulated with various concentrations of PDGF and PDGF/VEGF chimera for 3 hr at room temperature. PathHunter Detection Reagent (DiscoveRx) was added and incubated for 1 hr at room temperature to detect activated receptor. Chemiluminescence signal was analyzed on a microplate reader (BioTek Synergy H4).

Cell proliferation assay. BJ-5ta cells were seeded on 96-well plates at a density of 4,500 cells/well in complete media. After 24 hr, cells were replaced with serum-free media (SFM) containing 4 parts DMEM, 1 part M199, and 0.1% BSA for 24 hr. Cells were stimulated with PDGF-BB (Peprotech) and PDGF/VEGF chimera in SFM supplemented with 10 μg/ml insulin (Sigma). After 48 hr of growth, cell proliferation was measured using alamarBlue (Invitrogen) according to manufacturer's protocols. 10 μl of alamarBlue reagent was added to cells and incubated for 4 hr at 37° C. Fluorescence was measured on a microplate reader at an excitation wavelength of 570 nm and emission wavelength of 585 nm.

HUVECs were seeded on 96-well plates at a density of 7,500 cells/well in EGM BulletKit Media. After 24 hr, cells were replaced with serum-starvation media (SFM): EBM (Lonza CC-3121) with 0.2% BSA and 1% FBS for 12 hr. Cells were stimulated with VEGF₁₆₅ (Peprotech) and PDGF/VEGF chimera in SFM supplemented with 10 μg/ml insulin (Sigma). After 48 hr of growth, cell proliferation was measured using alamarBlue as described above.

### Example 5

To recombinantly express soluble PDGFR, we cloned the cDNA of residues 1-314 encoding the first three Ig-like domains of PDGFR-3 into the pAdd2 vector for HEK293 cell expression. Two different constructs were made: one with residues 1-314 attached to a C-terminal 6-His tag (PDGRβ-His), and one with residues 1-314 fused to the Fc region of mouse IgG2a (PDGRβ-Fc). The two constructs were expressed using the FreeStyle MAX 293 expression system. Both proteins were well expressed, with yields of over 1 mg protein from 60 ml of culture for PDGRβ-His and 120 ml of culture for PDGRβ-Fc.

A binding titration of PDGRβ-His and PDGRβ-Fc was performed on scPDGF proteins expressed on the surface of yeast. Bound protein was detected using fluorescently-labeled anti-His and anti-mouse-Fc antibodies. PDGRβ-Fc showed extremely tight binding with K_{d} of 28±2 pM (FIG. 33A). PDGRβ-His had much weaker binding and lower signal with a K_{d} of 310 nM (FIG. 33B), likely due to monovalent binding compared to PDGFR.

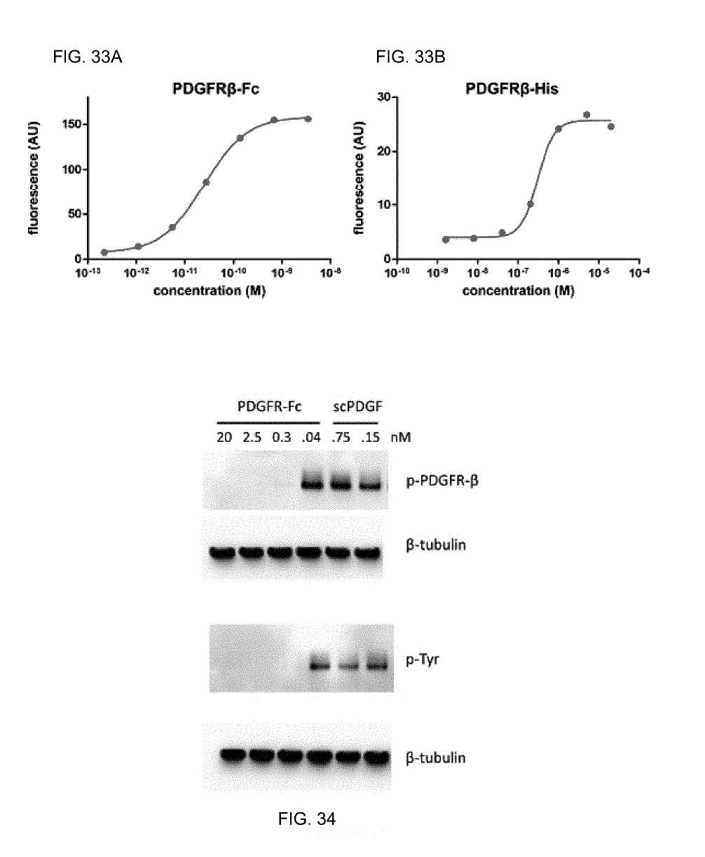

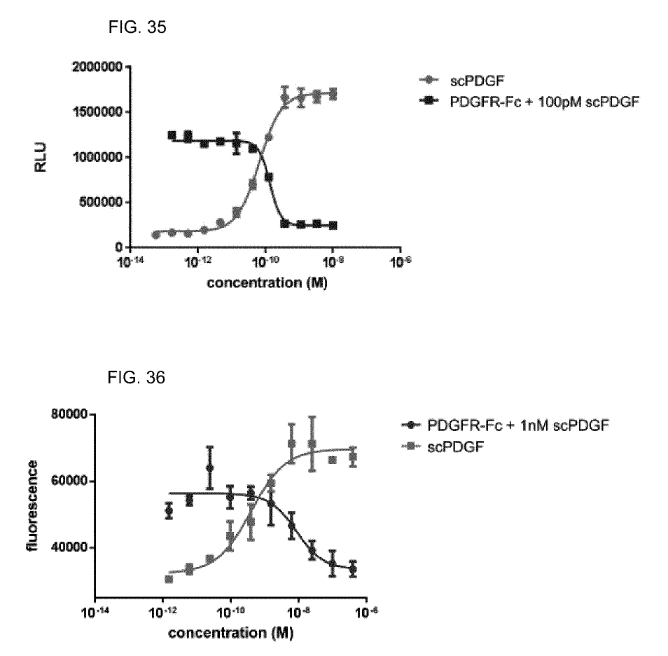

We also tested in a phosphorylation assay whether PDGRβ-Fc can be used as a ligand “trap” to bind PDGF in solution and neutralize its biological activity. PDGRβ-Fc strongly inhibited receptor phosphorylation by 150 pM scPDGF, showing complete inhibition at concentrations as low as 0.3 nM (FIG. 34). PDGRβ-Fc was tested in the DiscoveRx PathHunter reporter cell line. PDGRβ-Fc acted as an antagonist and inhibited stimulation by 100 pM of scPDGF with an IC₅₀ of 130 pM (FIG. 35). In a cell proliferation assay with NIH/3T3 cells, PDGRβ-Fc inhibits 1 nM scPDGF with an IC₅₀ of 8.7 nM (FIG. 36).

### Example 6

### Pterygium

Tissue specimens obtained from consenting patients undergoing clinically indicated pterygium removal surgery were subsequently tested for markers of angiogenic vasculature. Tissues were fixed in formalin before paraffin processing, embedding, and were sectioned at 5 pm onto Superfrost Plus slides. Pterygium tissue sections were deparaffinized in xylene and rehydrated through a graded alcohol series to water. The slides were subjected to heat-mediated antigen retrieval in sodium citrate buffer. Slides were washed 3×5 min in PBS, then incubated in 10% normal goat serum in PBS with 1% BSA for 3 hrs at RT for blocking. Each section was then incubated for 12 hrs at 4 C with a cocktail of two antibodies raised in differing species to achieve staining overlays. Antibodies for von Willebrand Factor (vWF), VEGFR2, CD31, and PDGFRβ were used. PBS with 1% BSA was used for all antibody dilutions. The slides were then washed 3×5 min in PBS, and incubated for 1 hr at RT in Alexa Fluor 488 and Alexa Fluor 594 conjugated antibodies raised in goat against mouse and rabbit, respectively. Slides were then washed 3×5 min in PBS, and mounted with 4′-6-diamidino-2-phenylindole (DAPI)-containing Vectashield mounting media. Fluorescence images were captured using a 10× Plan Apochromat objective on an Axiolmager Z1 Epifluorescence Microscope with appropriate filter sets. Exposure times for each antigen were constant across samples. All images of an antigen received the same linear brightness and contrast adjustments using Zen Blue software.

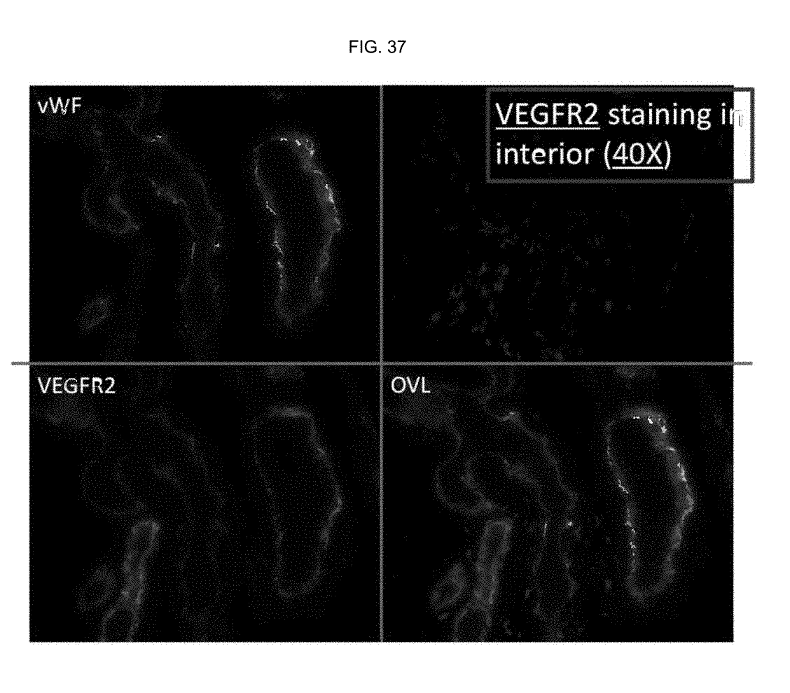

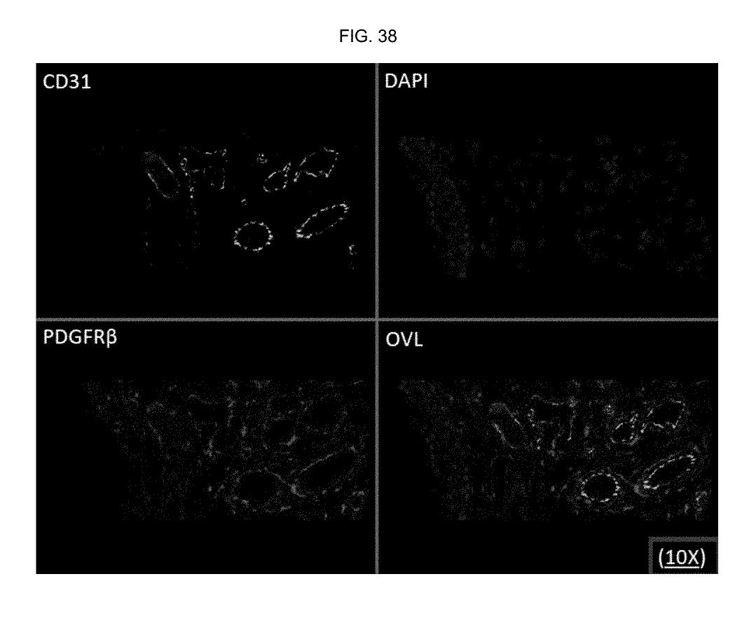

Shown in FIG. 37 are fluorescence images of von Willebrand Factor (vWF) and VEGFR2 staining, prominent staining with VEGFR2 was observed. Significantly, this staining co-localized with a known marker of endothelial cells (vWF) confirming that the expression of VEGFR2 is associated with endothelial cells. Shown in FIG. 38 are fluorescence images of CD31 and PDGFRβ staining. Prominent staining with PDGFRβ was observed. Significantly, this staining was found on cells surrounding endothelial cells (endothelial cells were identified by staining with a known marker, CD31); this staining pattern is consistent with the identity of PDGFRβ expressing cells being pericytes, which are known to closely associate with endothelial cells. In addition, PDGFRβ expression was also observed on cells that were not associated with vasculature.

Although the foregoing invention and its embodiments have been described in some detail by way of illustration and example for purposes of clarity of understanding, it is readily apparent to those of ordinary skill in the art in light of the teachings of this invention that certain changes and modifications may be made thereto without departing from the spirit or scope of the appended claims. Accordingly, the preceding merely illustrates the principles of the invention. It will be appreciated that those skilled in the art will be able to devise various arrangements which, although not explicitly described or shown herein, embody the principles of the invention and are included within its spirit and scope.

What is claimed is:

1. A hybrid polypeptide comprising:
a first chain HC1 comprised of alternating blocks of human PDGF amino acid sequence and human VEGF amino acid sequence; and a second chain HC2 comprised of alternating blocks of human VEGF amino acid sequence and human PDGF amino acid sequence;
wherein the hybrid polypeptide binds to PDGFR at one pole and VEGFR with one pole, thereby inhibiting signaling of both PDGFR and VEGFR.

2. A hybrid polypeptide according to claim 1, wherein the first chain HC1 is fused through a polypeptide linker to the second chain HC2.

3. The hybrid polypeptide of claim 1 or claim 2, wherein the polypeptide has a K_{d} of less than 100 nM for each of PDGFR and VEGFR.

4. The hybrid polypeptide of claim 1 or claim 2, wherein the polypeptide has a K_{d} of less than 10 nM for each of PDGFR and VEGFR.

5. The hybrid polypeptide of claim 1 or claim 2, wherein the polypeptide has a K_{d} of less than 5 nM for each of PDGFR and VEGFR.

6. The polypeptide of claim 1 or claim 2, wherein the HC1 polypeptide comprises cysteines residues at positions 10, 35, 41, 44, 45, 52, 86 and 88.

7. The polypeptide of claim 1 or claim 2, wherein the HC1 polypeptide comprises 5 alternating blocks of sequence, arranged as PDGF-B/VEGF-A/PDGF-B/VEGF-A/PDGF-B.

8. The polypeptide of claim 7 comprising the structure:
(a) first block (B1a) comprising from about 10-13 residues of PDGF-B, starting from residue 7 of the mature protein up to residue 16-19;
(b) second block of sequence (B1b) comprising from about 28-37 amino acid residues of VEGF-A, starting from residue 26-30 up to residue 57-62;
(c) third block (B1c) comprising from about 9-16 amino acid residues of PDGF-B, starting from residue 48-55 up to residue 63;
(d) fourth block (B1d) comprises from about 28-33 amino acid residues of VEGF-A, starting from residue 70-72 up to 99-102;
(e) fifth block (B1e) comprising from about 7-11 amino acid residues of PDGF-B, starting from residue 94-98 up to residue 104.

9. The polypeptide of claim 7 comprising the structure:
(a) first block (B1a) comprises from about 10-13 residues of PDGF-B, starting from residue 7 of the mature protein up to residue 16-19;
(b) second block of sequence (B1b) comprising from about 23-37 amino acid residues of VEGF-A, starting from residue 26-30 up to residue 52-62;
(c) third block (B1c) comprising from about 6-21 amino acid residues of PDGF-B, starting from residue 43-55 up to residue 60-63;
(d) fourth block (B1d) comprising from about 28-37 amino acid residues of VEGF-A, starting from residue 68-72 up to 99-104;
(e) fifth block (B1e) comprising from about 5-11 amino acid residues of PDGF-B, starting from residue 94-100 up to residue 104.

10. The polypeptide of claim 1 or claim 2, wherein the HC2 polypeptide comprises 5 alternating blocks of sequence, arranged as VEGF-A/PDGF-B/VEGF-A/PDGF-B/VEGF-A.

11. The polypeptide of claim 10 comprising the structure:
(a) first block (B2a) comprising from about 14-17 residues of VEGF-A, starting from residue 13 of the mature protein up to residue 26-29;
(b) second block of sequence (B2b) comprising from about 30-39 amino acid residues of PDGF-B, starting from residue 16-20 up to residue 49-54;
(c) third block (B2c) comprising from about 9-16 residues of VEGF-A, starting from residue 56-63;
(d) fourth block (B2d) comprising from about 31-36 amino acid residues of PDGF-B, starting from residue 62-64 up to residue 94-97;
(e) fifth block (B2e) comprising from about 7-11 residues of VEGF, starting from residue 99-103 up to residue 109.

12. The polypeptide of claim 10 comprising the structure:
(a) first block (B2a) comprising from about 14-17 residues of VEGF-A, starting from residue 13 of the mature protein up to residue 26-29;
(b) second block of sequence (B2b) comprising from about 25-39 amino acid residues of PDGF-B, starting from residue 16-20 up to residue 44-54;
(c) third block (B2c) comprising from about 6-21 residues of VEGF-A, starting from residue 51-63 up to residue 68-71;
(d) fourth block (B2d) comprising from about 31-40 amino acid residues of PDGF-B, starting from residue 60-64 up to residue 94-99;
(e) fifth block(B2e) comprising from about 5-11 residues of VEGF, starting from residue 99-105 up to residue 109.

13. The polypeptide of any one of claims 1-12 wherein VEGF-derived sequences of the hybrid polypeptide comprises at least one amino acid substitution selected from the group consisting of: V14A, V14I, V15A, K16R, F17L, M18R, D19G, Q22R, R23K, I29F, I29V, L32S, I35V, F36L, F36S, D41N, E42K, E44G, Y45H, F471, F47S, K48E, P49L, S50P, P53S, G58S, C60Y, D63H, D63N, D63G, E67G, I76T, M78V, Q79H, I80V, M81T, M81V, R82G, H86Y, Q87R, Q89H, H9OR, I91T, I91V, G92D, S95T, N100D, K101E, E103V, K107R, D109V with reference to SEQ ID NO: 2.

14. The polypeptide of any one of claims 1-12 wherein VEGF-derived sequences of the hybrid polypeptide comprises at least one amino acid substitution selected from the group consisting of: K16R, Y21H, I29M, I29V, L32S, D41N, F47S, V69A, M81I, K84T, I91F, L97F, D109V with reference to SEQ ID NO: 2.

15. The polypeptide of any one of claims 1-12 wherein VEGF-derived sequences of the hybrid polypeptide comprises at least one amino acid substitution selected from the group consisting of: T71A, Q79R, I83V with reference to SEQ ID NO: 2.

16. The polypeptide of any one of claims 1-12 wherein PDGF-derived sequences of the hybrid polypeptide comprises at least one amino acid substitution selected from the group consisting of: E9G, I13F, R28G, I30V, V39A, V72A, I77F, K8OR, L955, K98R with reference to SEQ ID NO: 1.

17. The polypeptide of any one of claims 1-12 wherein PDGF-derived sequences of the hybrid polypeptide comprises at least one amino acid substitution selected from the group consisting of: A8V, M12V, T18A, I25V, R56G, N57S, Q59R, I77T, K80R, F84L, A87T, A96T K98I, T101A with reference to SEQ ID NO: 1.

18. The polypeptide of any one of claims 1-12 wherein PDGF-derived sequences of the hybrid polypeptide comprises at least one amino add substitution selected from the group consisting of: R56K, T63A, T88S, E100V, E100R with reference to SEQ ID NO: 1.

19. The polypeptide of any one of claims 1-12 wherein PDGF-derived sequences of the hybrid polypeptide comprises at least one amino acid substitution selected from the group consisting of: R32A with reference to SEQ ID NO: 1.

20. An isolated polypeptide comprised of alternating blocks of human PDGF-B amino acid sequence and human VEGF-A amino acid sequence; fused through a polypeptide linker to a second chain HC2 comprised of alternating blocks of human VEGF-A amino acid sequence and human PDGF-B amino acid sequence, wherein the HC1 polypeptide has at least 90% sequence identity with any one of SEQ ID NO: 3, residues 1-93; and SEQ ID NO: 8, 10, 12, 14, 16, 18, 20, 22 and 24.

21. An isolated polypeptide comprised of alternating blocks of human PDGF-B amino acid sequence and human VEGF-A amino acid sequence; fused through a polypeptide linker to a second chain HC2 comprised of alternating blocks of human VEGF-A amino acid sequence and human PDGF-B amino acid sequence, wherein the HC1 polypeptide has at least 95% sequence identity with any one of SEQ ID NO: 3, residues 1-93; and SEQ ID NO: 8, 10, 12, 14, 16, 18, 20, 22 and 24.

22. The polypeptide of claim 20 or 21, wherein the HC1 polypeptide comprises the sequence set forth in any one of SEQ ID NO: 3, residues 1-93; and SEQ ID NO: 8, 10, 12, 14, 16, 18, 20, 22 and 24.

23. The polypeptide of claim 20 or 21, wherein the HC2 polypeptide has at least 90% sequence identity with any one of SEQ ID NO: 3, residues 108-209; and SEQ ID NO: 9, 11, 13, 15, 17, 19, 21, 23 and 25.

24. The polypeptide of of claim 20 or 21, wherein the HC2 polypeptide has at least 95% sequence identity with any one of SEQ ID NO: 3, residues 108-209; and SEQ ID NO: 9, 11, 13, 15, 17, 19, 21, 23 and 25.

25. The polypeptide of claim 20 or 21, wherein the HC2 polypeptide comprises the sequence set forth in any one of SEQ ID NO: 3, residues 108-209; and SEQ ID NO: 9, 11, 13, 15, 17, 19, 21, 23 and 25.

26. The polypeptide of claim 20 or 21, wherein the the HC1 polypeptide has at least 95% sequence identity with any one of SEQ ID NO: 3, residues 1-93; and SEQ ID NO: 8, 10, 12, 14, 16, 18, 20, 22 and 24 and the HC2 polypeptide has at least 95% sequence identity with any one of SEQ ID NO: 3, residues 108-209; and SEQ ID NO: 9, 11, 13, 15, 17, 19, 21, 23 and 25, fused through a polypeptide linker of 5-25 amino acids in length.

27. The polypeptide of claim 2 or claim 26, wherein the flexible linker comprises the sequence (GGGGS)n, where n is from 1-5, or GSTSGSGKSSEGKG.

28. A hybrid polypeptide according to any one of claims 1-27, further comprising a functional conjugate.

29. The hybrid polypeptide according to claim 28, wherein said functional conjugate is a detectable moiety.

30. A pharmaceutical composition comprising a hybrid polypeptide according to any one of claims 1-29, and a pharmaceutically acceptable excipient.

31. An isolated nucleic acid encoding a hybrid polypeptide according to any one of claims 1-29.

32. A method of concomitant inhibition of VEGFR and PDGFR, the method comprising contacting a cell or population of cells with an effective dose of a pharmaceutical composition according to claim 30, or an isolated nucleic acid according to claim 31.

33. A method of inhibiting angiogenesis, the method comprising:
contacting endothelial cells and/or perivascular cells associated with said angiogenesis with an effective dose of a pharmaceutical composition according to claim 30, or an isolated nucleic acid according to claim 31.

34. The method of claim 32 or 33 wherein said contacting is performed in vivo.

35. The method of claim 34, wherein said angiogenesis is associated with a vascularized tumor.

36. The method of claim 35, wherein said angiogenesis is associated with psoriasis.

37. The method of claim 35, wherein said angiogenesis is associated with age-related macular degeneration.

38. The method of claim 35, wherein said angiogenesis is associated with diabetic retinopathy.

39. The method of claim 35, wherein said angiogenesis is associated with rheumatoid arthritis.

40. A method for imaging tissue, comprising contacting a tissue with a hybrid polypeptide of claim 29.

41. A method for diagnosing the presence of one or more cancerous cells in a tissue, comprising contacting a tissue with a hybrid polypeptide of claim 29.

42. A method for monitoring the progression of one or more cancerous cells in a tissue, comprising contacting a tissue with a hybrid polypeptide of claim 29.

43. An isolated variant PDGF polypeptide having enhanced affinity for PDGFR, and comprising at least one amino acid substitution relative to wild-type PDGF.

44. The polypeptide of claim 33, comprising one or both of amino acid substitutions E9G, I13F, R28G, I30V, V39A, V72A, I77F, K8OR, L95S, K98R with reference to SEQ ID NO: 1.

45. The polypeptide of claim 1, fused or otherwise joined to an immunoglobulin sequence to form a chimeric protein.

46. The polypeptide of claim 45, wherein the immunoglobulin sequence comprises an immunoglobulin constant domain sequence.

47. A polypeptide comprising the first three Ig-like domains of PDGFR-β fused to an Fc sequence.

48. A method of non-surgically treating a disorder characterized by neovascularization of the external surface of an eye, including the cornea and bulbar conjunctiva, of a subject in need thereof, comprising administering to the subject an effective amount of a pharmaceutical composition comprising a composition of claim 30.

49. A method for preventing recurrence of neovascularization of the external surface of an eye, including the cornea and bulbar conjunctiva, of a subject in need thereof, comprising administering to the subject an effective amount of a pharmaceutical composition comprising a composition of claim 30

50. The method of any one of claim 48 or 49, wherein the pharmaceutical composition is formulated as an ophthalmically acceptable solution, cream or ointment.

51. The method of any one of claims 48-50 wherein the disorder characterized by neovascularization of the external surface of the eye is pterygium.


SEQUENCE LISTING (25 sequences)

SEQ ID NO: 1 - protein, 109 aa, Homo sapiens
SLGSLTIAEPAMIAECKTRTEVFEISRRLIDRTNANFLVWPPCVEVQRCSGCCNNRNVQCRPTQVQLRPVQVRKIEIVRKKPIFKKATVTLEDHLACKCETVAAARPVT

SEQ ID NO: 2 - protein, 109 aa, Homo sapiens
APMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKD

SEQ ID NO: 3 - protein, 209 aa, Homo sapiens
IAEPAMIAECKTRETLVDIFQEYPDEIEYIFKPSCVPLMRCSGCCNNRNVQCRPTEESNITMQIMRIKPHQGQHIGEMSFLQHLACKCETVAAGSTSGSGKSSEGKGEVVKFMDVYQRSYCHPITEVFEISRRLIDRTNANFLVWPPCVEVQRCGGCCNDEGLECVPTQVQLRPVQVRKIEIVRKKPIFKKATVTLEDHNKCECRPKKD

SEQ ID NO: 4 - protein, 209 aa, Homo sapiens
IAEPAMIAECKTRTELVDIFQEYPDEIEYIFKPSCVPLMRCSGCCNNRNVQCRPTEESNITMQIMRIKPHQGQHIGEMSFLQHLACKCETVAAGSTSGSGKSSEGKGEVVKFMDVYQRSYCHPIETVFEISRRLIDRTNANFLVWPPCVEVQRCGGCCNDEGLECVPTQVQLRPVQVRKIEIVRKKPIFKKATVTLEDHNKCECRPKKD

SEQ ID NO: 5 - protein, 209 aa, Homo sapiens
IAEPAMIAECKTRTEVVDIFQEYPDEIEYIFKPSCVPLMRCSGCCNNRNVQCRPTEESNITMQIMRIKPHQGQHIGEMSFLQHLACKCETVAAGSTSGSGKSSEGKGEVVKFMDVYQRSYCHPIETLFEISRRLIDRTNANFLVWPPCVEVQRCGGCCNDEGLECVPTQVQLRPVQVRKIEIVRKKPIFKKATVTLEDHNKCECRPKKD

SEQ ID NO: 6 - protein, 209 aa, Homo sapiens
IAEPAMIAECKTRTEVFDIFQEYPDEIEYIFKPSCVPLMRCSGCCNNRNVQCRPTEESNITMQIMRIKPHQGQHIGEMSFLQHLACKCETVAAGSTSGSGKSSEGKGEVVKFMDVYQRSYCHPIETLVEISRRLIDRTNANFLVWPPCVEVQRCGGCCNDEGLECVPTQVQLRPVQVRKIEIVRKKPIFKKATVTLEDHNKCECRPKKD

SEQ ID NO: 7 - protein, 209 aa, Homo sapiens
IAEPAMIAECKTRTEVFEIFQEYPDEIEYIFKPSCVPLMRCSGCCNNRNVQCRPTEESNITMQIMRIKPHQGQHIGEMSFLQHLACKCETVAAGSTSGSGKSSEGKGEVVKFMDVYQRSYCHPIETLVDISRRLIDRTNANFLVWPPCVEVQRCGGCCNDEGLECVPTQVQLRPVQVRKIEIVRKKPIFKKATVTLEDHNKCECRPKKD

SEQ ID NO: 8 - protein, 93 aa, Homo sapiens
IAEPAMIAECHPIETLVDIFQEYPDEIEYIFKPSCVPLMRCGGCCNNRNVQCRPTEESNITMQIMRIKPHQGQHIGEMSFLQHNKCKCETVAA

SEQ ID NO: 9 - protein, 102 aa, Homo sapiens
EVVKFMDVYQRSYCKTRTEVFEISRRLIDRTNANFLVWPPCVEVQRCSGCCNDEGLECVPTQVQLRPVQVRKIEIVRKKPIFKKATVTLEDHLACKCRPKKD

SEQ ID NO: 10 - protein, 93 aa, Homo sapiens
IAEPAMIAECKTRETLVDIFQEYPDEIEYIFKPSCVPLMRCSGCCNNRNVQCRPTEESNITMQIMRIKPHQGQHIGEMSFLQHNKCKCETVAA

SEQ ID NO: 11 - protein, 102 aa, Homo sapiens
EVVKFMDVYQRSYCHPITEVFEISRRLIDRTNANFLVWPPCVEVQRCGGCCNDEGLECVPTQVQLRPVQVRKIEIVRKKPIFKKATVTLEDHLACECRPKKD

SEQ ID NO: 12 - protein, 93 aa, Homo sapiens
IAEPAMIAECKTIETLVDIFQEYPDEIEYIFKPSCVPLMRCSGCCNNRNVQCRPTEESNITMQIMRIKPHQGQHIGEMSFLQHLACKCETVAA

SEQ ID NO: 13 - protein, 102 aa, Homo sapiens
EVVKFMDVYQRSYCHPRTEVFEISRRLIDRTNANFLVWPPCVEVQRCGGCCNDEGLECVPTQVQLRPVQVRKIEIVRKKPIFKKATVTLEDHNKCECRPKKD

SEQ ID NO: 14 - protein, 93 aa, Homo sapiens
IAEPAMIAECHPIETLVDIFQEYPDEIEYIFKPSCVPLMRCGGCCNNRNVQCRPTEESNITMQIMRIKPHQGQHIGEMSFLQHNKCRCETVAA

SEQ ID NO: 15 - protein, 102 aa, Homo sapiens
EVVKFMDVYQRSYCKTRTEVFEISRRLIDRTNANFLVWPPCVEVQRCSGCCNDEGLECVPTQVQLRPVQVRKIEFVRKKPIFKKATVTLEDHLACECRPKKD

SEQ ID NO: 16 - protein, 93 aa, Homo sapiens
IAEPAVIAECHPIETLVDILQEYPDEIGYIFKPSCVPLMRCGGCCNNRNVQCRPTEESNITMQIMRIKPHRGQHIGEMSFLQHNKCKCETVAA

SEQ ID NO: 17 - protein, 102 aa, Homo sapiens
EVVKFMDVYQRSYCKTRTEVFEISRRLIDRTNANFLVWPPCVEVQRCSGCCNHEGLECVPTQVQLRPVQVRKIEIVRKKPIFKKATVTLEDHLACECRPKKD

SEQ ID NO: 18 - protein, 93 aa, Homo sapiens
IAEPAMIAECHPIETLVDILQEYPDEIGYIFKPSCVPLMRCGGCCNNRNVQCRPTEESNITMQIMRIKPHRGQHIGEMSFLQHNKCKCETVAA

SEQ ID NO: 19 - protein, 102 aa, Homo sapiens; identical to SEQ ID NO: 17

SEQ ID NO: 20 - protein, 93 aa, Homo sapiens; identical to SEQ ID NO: 16

SEQ ID NO: 21 - protein, 102 aa, Homo sapiens
EVVKFMDVYQRSYCKTRTEVFEISRRLIDRTNANFLVWPPCVEVQRCSGCCNHEGLECVPTQVQLRPVQVRKIEIVRKKPIFKKATVTLEDHLACECRPKKV

SEQ ID NO: 22 - protein, 93 aa, Homo sapiens
IAEPAMIAECHPIETLVDILQEYPDEIGYIFKPSCVPLMRCGGCCNNRNVQCRPTEESNITMQIMRIKPHRGQHIGEMSFLQHNKCRCETVAA

SEQ ID NO: 23 - protein, 102 aa, Homo sapiens
EVVKFMDVYQRSYCKTRTEVFEISRRLIDRTNANFLVWPPCVEVQRCSGCCNHEGLECVPTQVQLRPVQVRKIEFVRKKPIFKKATVTLEDHLACECRPKKD

SEQ ID NO: 24 - protein, 93 aa, Homo sapiens; identical to SEQ ID NO: 22

SEQ ID NO: 25 - protein, 102 aa, Homo sapiens
EVVKFMDVYQRSYCKTRTEVFEISRRLIDATNANFLVWPPCVEVQRCSGCCNHEGLECVPTQVQLRPVQVRKIEFVRKKPIFKKATVTLEDHLACECRPKKD